(19) United States
(12) Patent Application Publication
(10) Pub. No.: US 2019/0307890 A1
(43) Pub. Date: Oct. 10, 2019

(54) PYRROLOBENZODIAZEPINE CONJUGATES

(71) Applicant: MEDIMMUNE LIMITED, Cambridge, Cambridgeshire (GB)

(72) Inventors: Philip Wilson HOWARD, Cambridge, Cambridgeshire (GB); Thais CAILLEAU, Cambridge, Cambridgeshire (GB)

(21) Appl. No.: 16/349,228

(22) PCT Filed: Nov. 17, 2017
(86) PCT No.: PCT/EP2017/079572

§ 371 Date: May 10, 2019

(30) Foreign Application Priority Data
Nov. 17, 2016 (GB) 1619490.4

(51) Int. Cl. A61K 47/68; C07K 5/062; A61K 31/5517; A61P 35/00

(52) U.S. Cl. CPC A61K 47/6803; C07K 5/06052; A61P 35/00; A61K 31/5517; A61K 47/6863; A61K 47/6855

(57) ABSTRACT

A conjugate of formula (I):L-(D^{L})ₚ, wherein L is a Ligand unit, D is a Drug Linker unit of formula IIa, IIb or IIc, wherein p is an integer of from 1 to 20.

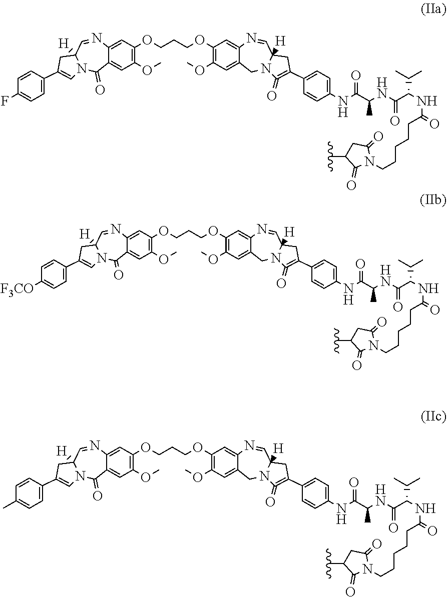

The present invention relates to conjugates comprising specific pyrrolobenzodiazepines (PBD), and the precursor drug linker used to make such conjugates.

### BACKGROUND TO THE INVENTION

Some pyrrolobenzodiazepines (PBDs) have the ability to recognise and bond to specific sequences of DNA; the preferred sequence is PuGPu. The first PBD antitumour antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and over 10 synthetic routes have been developed to a variety of analogues (Thurston, et al., Chem. Rev. 1994, 433-465 (1994)). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102)(Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)) and tomamycin (Arima, et al., J. Antibiotics, 25, 437-444 (1972)). PBDs are of the general structure:

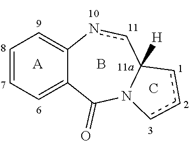

They differ in the number, type and position of substituents, in both their aromatic A rings and pyrrolo C rings, and in the degree of saturation of the C ring. In the B-ring there is either an imine (N═C), a carbinolamine(NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDeventer, Acc. Chem. Res., 19, 230-237 (1986)). Their ability to form an adduct in the minor groove, enables them to interfere with DNA processing, hence their use as antitumour agents.

It has been previously disclosed that the biological activity of this molecules can be potentiated by joining two PBD units together through their C8/C′-hydroxyl functionalities via a flexible alkylene linker (Bose, D. S., et al., J. Am. Chem. Soc., 114, 4939-4941 (1992); Thurston, D. E., et al., J. Org. Chem., 61, 8141-8147 (1996)). The PBD dimers are thought to form sequence-selective DNA lesions such as the palindromic 5′-Pu-GATC-Py-3′ interstrand cross-link (Smellie, M., et al., Biochemistry, 42, 8232-8239 (2003); Martin, C., et al., Biochemistry, 44, 4135-4147) which is thought to be mainly responsible for their biological activity.

One example of a PBD dimer is SG2000 (SJG-136):

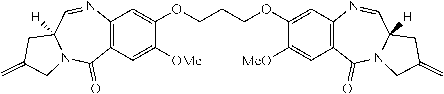

(Gregson, S., et al., J. Med. Chem., 44, 737-748 (2001); Alley, M. C., et al., Cancer Research, 64, 6700-6706 (2004); Hartley, J. A., et al., Cancer Research, 64, 6693-6699 (2004)) which has been involved in clinical trials as a standalone agent, for example, NCT02034227 investigating its use in treating Acute Myeloid Leukemia and Chronic Lymphocytic Leukemia (see: https://www.clinicaltrials.gov/ct2/show/NCT02034227).

Dimeric PBD compounds bearing C2 aryl substituents, such as SG2202 (ZC-207), are disclosed in WO 2005/085251:

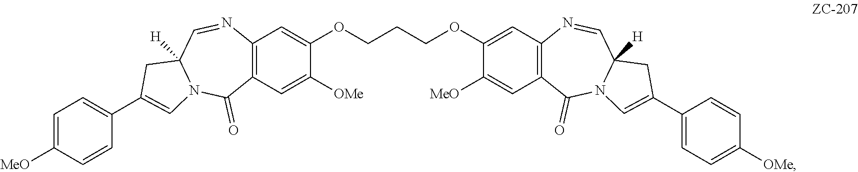 [SMILES: COc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

and in WO2006/111759, bisulphites of such PBD compounds, for example SG2285 (ZC-423):

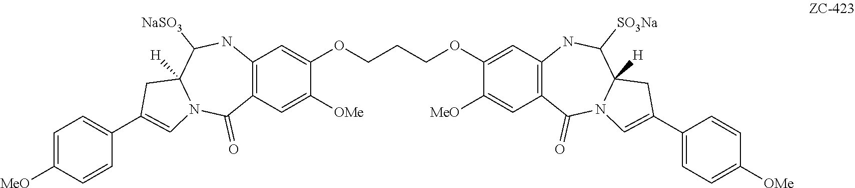

These compounds have been shown to be highly useful cytotoxic agents (Howard, P. W., et al., Bioorg. Med. Chem. (2009), doi: 10.1016/j.bmcl.2009.09.012).

WO2010/043880 discloses PBD dimers bearing a anchor for the attachment of another moiety, and WO2011/130613 discloses the use of such modified PBD dimers in antibody drug conjugates. One of the conjugates disclosed, that comprising:

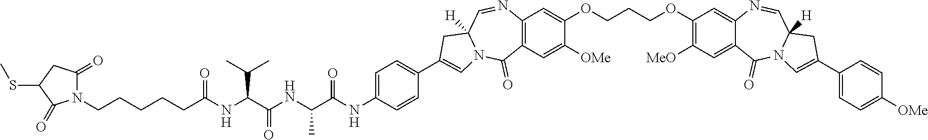 [SMILES: COc1ccc(C2=CN3C(=O)c4cc(OC)c(OCCCOc5cc6c(cc5OC)C(=O)N5C=C(c7ccc(NC(=O)[C@H](C)NC(=O)[C@@H](NC(=O)CCCCCN8C(=O)CC(SC)C8=O)C(C)C)cc7)C[C@H]5C=N6)cc4N=C[C@@H]3C2)cc1]

(termed SGN-CD33A) is undergoing clinical trials in patients with CD33—Postiive Acute Myeloid Leukemia (AML). The pre-clinical development of SGN-CD33A is described in Kung Sutherland, M., et al., Blood, 2013; 122(8) 1455-1463 (doi:10.1182/blood-2013-03-491506). An initial report of the clinical trials was provided at the 56^{th} ASH Annual Meeting, 6-9 Dec. 2014 (Stein, E., et al., Session 616 -Abstract 623).

More recent publications have described the following conjugates comprising PBD dimers, for example, WO2014/057073 describes:

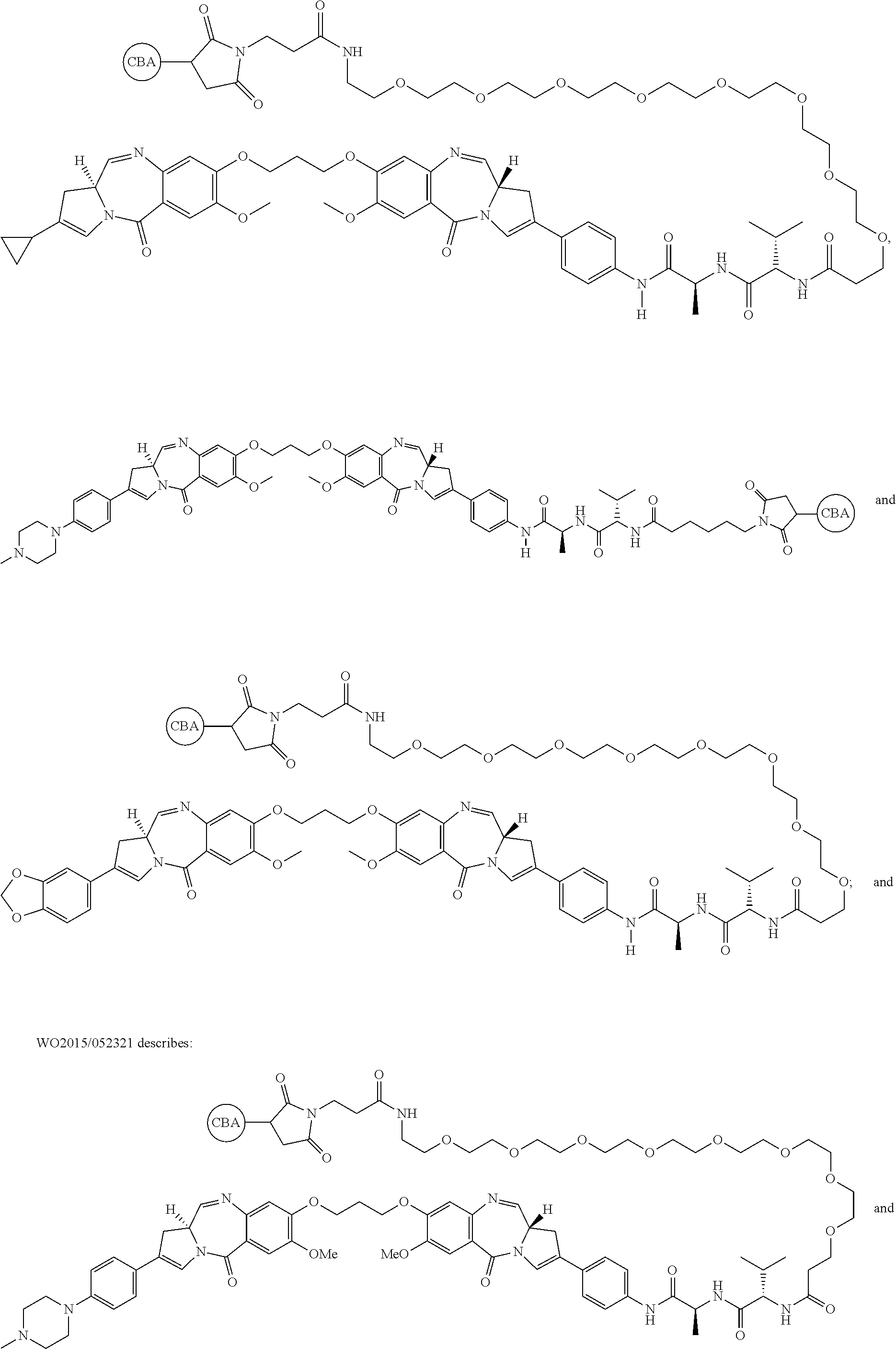

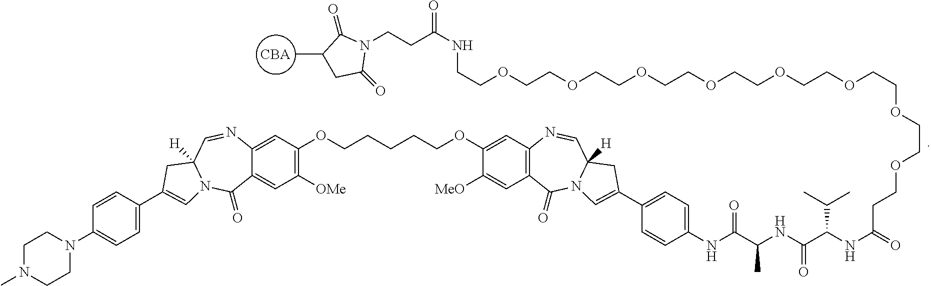 [SMILES: COc1cc2c(cc1OCCCCCOc1cc3c(cc1OC)C(=O)N1C=C(c4ccc(N5CCN(C)CC5)cc4)C[C@H]1C=N3)N=C[C@@H]1CC(c3ccc(NC(=O)[C@H](C)NC(=O)[C@@H](NC(=O)CCOCCOCCOCCOCCOCCOCCOCCOCCNC(=O)CCN4C(=O)CC(C)C4=O)C(C)C)cc3)=CN1C2=O]

### DISCLOSURE OF THE INVENTION

The present inventors have developed further drug linkers and conjugates comprising said drug linkers. These drug linker replace the methoxy group in the drug linker present in SGN-CD33A with either a fluoro, trifluoromethoxy or methyl group. Such groups will increase the Log P of the drug linkers.

Such an increase in Log P would be expected to make the drug linker and its conjugates more hydrophobic and more aggregative. In addition, the increase in hydrophobicity would be expected to lead to more rapid clearance in vivo, and hence a reduced active. Surprisingly, this does not appear to be the case in vivo for at least some of the conjugates herein.

Furthermore, the conjugates of the newly developed drug linker appear to show a reduced activity in vitro to those including the drug linker from SGN-CD33A, which would discourage further testing, but a surprising comparable in vivo activity, for at least some of the conjugates herein.

In a first aspect, the present invention provides Conjugates of formula I:

L-(D^{L})ₚ (I)

wherein Lisa Ligand unit (i.e., a targeting agent), D is a Drug Linker unit of formula IIa, IIb or IIc:

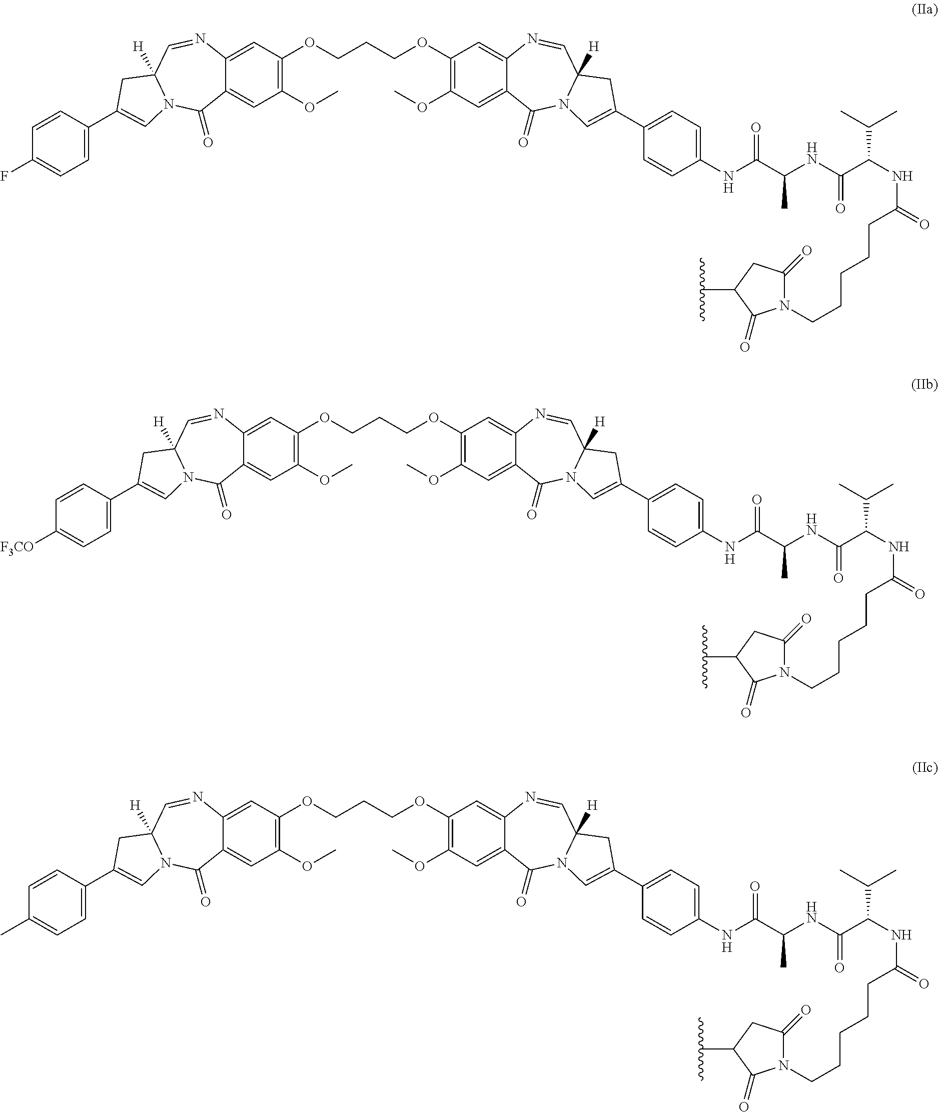

wherein p is an integer of from 1 to 20.

The Ligand unit, described more fully below, is a targeting agent that binds to a target moiety. The Ligand unit can, for example, specifically bind to a cell component (a Cell

Binding Agent) or to other target molecules of interest. The Ligand unit can be, for example, a protein, polypeptide or peptide, such as an antibody, an antigen-binding fragment of an antibody, or other binding agent, such as an Fc fusion protein.

A second aspect of the present invention provides a compound of formula IIIa, IIIb or IIIc:

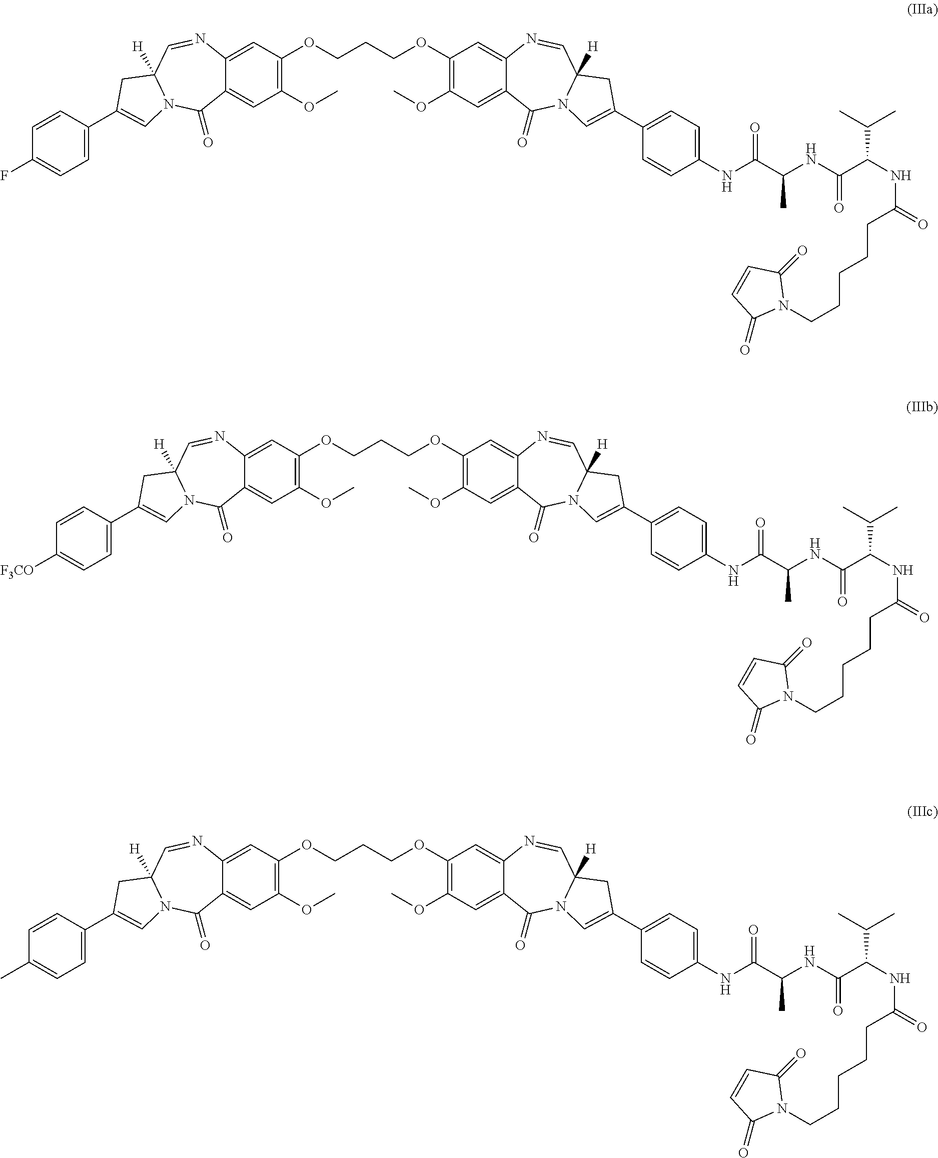 [SMILES: COc1cc2c(cc1OCCCOc1cc3c(cc1OC)C(=O)N1C=C(c4ccc(NC(=O)[C@H](C)NC(=O)[C@@H](NC(=O)CCCCCN5C(=O)C=CC5=O)C(C)C)cc4)C[C@H]1C=N3)N=C[C@@H]1CC(c3ccc(F)cc3)=CN1C2=O ; COc1cc2c(cc1OCCCOc1cc3c(cc1OC)C(=O)N1C=C(c4ccc(OC(F)(F)F)cc4)C[C@H]1C=N3)N=C[C@@H]1CC(c3ccc(NC(=O)[C@H](C)NC(=O)[C@@H](NC(=O)CCCCCN4C(=O)C=CC4=O)C(C)C)cc3)=CN1C2=O ; COc1cc2c(cc1OCCCOc1cc3c(cc1OC)C(=O)N1C=C(c4ccc(NC(=O)[C@H](C)NC(=O)[C@@H](NC(=O)CCCCCN5C(=O)C=CC5=O)C(C)C)cc4)C[C@H]1C=N3)N=C[C@@H]1CC(c3ccc(C)cc3)=CN1C2=O]

A third aspect of the present invention provides the use of a conjugate of the first aspect of the invention in the manufacture of a medicament for treating a proliferative disease. The third aspect also provides a conjugate of the first aspect of the invention for use in the treatment of a proliferative disease. The third aspect also provides a method of treating a proliferative disease comprising administering a therapeutically effective amount of a conjugate of the first aspect of the invention to a patient in need thereof.

One of ordinary skill in the art is readily able to determine whether or not a candidate conjugate treats a proliferative condition for any particular cell type. For example, assays which may conveniently be used to assess the activity offered by a particular compound are described in the examples below.

A fourth aspect of the present invention provides the synthesis of a conjugate of the first aspect of the invention comprising conjugating an appropriate compound (drug linker) of the second aspect of the invention with a Ligand Unit

### BRIEF DESCRIPTION OF FIGURES

FIG. 1 shows the effect on volume of NCI-N87 tumours following treatment with conjugates of the present invention;

FIG. 2 shows the effect on volume of NCI-N87 tumours following treatment with a conjugate of the present invention;

FIG. 3 shows the effect on volume of a JIMT-1 tumour following treatment with a conjugate of the present invention.

Ligand Unit

The Ligand Unit may be of any kind, and include a protein, polypeptide, peptide and a non-peptidic agent that specifically binds to a target molecule. In some embodiments, the Ligand unit may be a protein, polypeptide or peptide. In some embodiments, the Ligand unit may be a cyclic polypeptide. These Ligand units can include antibodies or a fragment of an antibody that contains at least one target molecule-binding site, lymphokines, hormones, growth factors, or any other cell binding molecule or substance that can specifically bind to a target.

The terms “specifically binds” and “specific binding” refer to the binding of an antibody or other protein, polypeptide or peptide to a predetermined molecule (e.g., an antigen). Typically, the antibody or other molecule binds with an affinity of at least about 1×10⁷ M⁻¹, and binds to the predetermined molecule with an affinity that is at least two-fold greater than its affinity for binding to a non-specific molecule (e.g., BSA, casein) other than the predetermined molecule or a closely-related molecule.

Examples of Ligand units include those agents described for use in WO 2007/085930, which is incorporated herein.

In some embodiments, the Ligand unit is a Cell Binding Agent that binds to an extracellular target on a cell. Such a Cell Binding Agent can be a protein, polypeptide, peptide or a non-peptidic agent. In some embodiments, the Cell Binding Agent may be a protein, polypeptide or peptide. In some embodiments, the Cell Binding Agent may be a cyclic polypeptide. The Cell Binding Agent also may be antibody or an antigen-binding fragment of an antibody. Thus, in one embodiment, the present invention provides an antibody-drug conjugate (ADC).

Cell Binding Agent

A cell binding agent may be of any kind, and include peptides and non-peptides. These can include antibodies or a fragment of an antibody that contains at least one binding site, lymphokines, hormones, hormone mimetics, vitamins, growth factors, nutrient-transport molecules, or any other cell binding molecule or substance.

Peptides

In one embodiment, the cell binding agent is a linear or cyclic peptide comprising 4-30, preferably 6-20, contiguous amino acid residues. In this embodiment, it is preferred that one cell binding agent is linked to one monomer or dimer pyrrolobenzodiazepine compound.

In one embodiment the cell binding agent comprises a peptide that binds integrin αᵥβ_{6.} The peptide may be selective for αᵥβ₆ over XYS.

In one embodiment the cell binding agent comprises the A20FMDV-Cys polypeptide. The A20FMDV-Cys has the sequence: NAVPNLRGDLQVLAQKVARTC. Alternatively, a variant of the A20FMDV-Cys sequence may be used wherein one, two, three, four, five, six, seven, eight, nine or ten amino acid residues are substituted with another amino acid residue. Furthermore, the polypeptide may have the sequence NAVXXXXXXXXXXXXXXXRTC.

Antibodies

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)_{2,} and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J.Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising a VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, β, ϵ, γ, and μ, respectively. The subunit structures and three-dimensional configurations of different classes of immunoglobulins are well known.

Humanisation

Techniques to reduce the in vivo immunogenicity of a non-human antibody or antibody fragment include those termed “humanisation”.

A “humanized antibody” refers to a polypeptide comprising at least a portion of a modified variable region of a human antibody wherein a portion of the variable region, preferably a portion substantially less than the intact human variable domain, has been substituted by the corresponding sequence from a non-human species and wherein the modified variable region is linked to at least another part of another protein, preferably the constant region of a human antibody. The expression “humanized antibodies” includes human antibodies in which one or more complementarity determining region (“CDR”) amino acid residues and/or one or more framework region (“FW” or “FR”) amino acid residues are substituted by amino acid residues from analogous sites in rodent or other non-human antibodies. The expression “humanized antibody” also includes an immunoglobulin amino acid sequence variant or fragment thereof that comprises an FR having substantially the amino acid sequence of a human immunoglobulin and a CDR having substantially the amino acid sequence of a non-human immunoglobulin.

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g. murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins.

There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

CDR Grafting

In this technique, the humanized antibodies are human immunoglobulins (recipient antibody) in which residues from a complementary-determining region (CDR) of the recipient antibody are replaced by residues from a CDR of a non-human species (donor antibody) such as mouse, rat, camel, bovine, goat, or rabbit having the desired properties (in effect, the non-human CDRs are ‘grafted’ onto the human framework). In some instances, framework region (FR) residues of the human immunoglobulin are replaced by corresponding non-human residues (this may happen when, for example, a particular FR residue has significant effect on antigen binding).

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin.

Guided Selection

The method consists of combining the VH or VL domain of a given non-human antibody specific for a particular epitope with a human VH or VL library and specific human V domains are selected against the antigen of interest. This selected human VH is then combined with a VL library to generate a completely human VH×VL combination. The method is described in Nature Biotechnology (N.Y.) 12, (1994) 899-903.

Composite Antibodies

In this method, two or more segments of amino acid sequence from a human antibody are combined within the final antibody molecule. They are constructed by combining multiple human VH and VL sequence segments in combinations which limit or avoid human T cell epitopes in the final composite antibody V regions. Where required, T cell epitopes are limited or avoided by, exchanging V region segments contributing to or encoding a T cell epitope with alternative segments which avoid T cell epitopes. This method is described in US 2008/0206239 A1.

Deimmunization

This method involves the removal of human (or other second species) T-cell epitopes from the V regions of the therapeutic antibody (or other molecule). The therapeutic antibodies V-region sequence is analysed for the presence of MHC class II-binding motifs by, for example, comparison with databases of MHC-binding motifs (such as the “motifs” database hosted at www.wehi.edu.au). Alternatively, MHC class II-binding motifs may be identified using computational threading methods such as those devised by Altuvia et al. (J. Mol. Biol. 249 244-250 (1995)); in these methods, consecutive overlapping peptides from the V-region sequences are testing for their binding energies to MHC class II proteins. This data can then be combined with information on other sequence features which relate to successfully presented peptides, such as amphipathicity, Rothbard motifs, and cleavage sites for cathepsin B and other processing enzymes.

Once potential second species (e.g. human) T-cell epitopes have been identified, they are eliminated by the alteration of one or more amino acids. The modified amino acids are usually within the T-cell epitope itself, but may also be adjacent to the epitope in terms of the primary or secondary structure of the protein (and therefore, may not be adjacent in the primary structure). Most typically, the alteration is by way of substitution but, in some circumstances amino acid addition or deletion will be more appropriate.

All alterations can be accomplished by recombinant DNA technology, so that the final molecule may be prepared by expression from a recombinant host using well established methods such as Site Directed Mutagenesis. However, the use of protein chemistry or any other means of molecular alteration is also possible.

Resurfacing

This method involves:
(a) determining the conformational structure of the variable region of the non-human (e.g. rodent) antibody (or fragment thereof) by constructing a three-dimensional model of the non-human antibody variable region;
(b) generating sequence alignments using relative accessibility distributions from x-ray crystallographic structures of a sufficient number of non-human and human antibody variable region heavy and light chains to give a set of heavy and light chain framework positions wherein the alignment positions are identical in 98% of the sufficient number of non-human antibody heavy and light chains;
(c) defining for the non-human antibody to be humanized, a set of heavy and light chain surface exposed amino acid residues using the set of framework positions generated in step (b);
(d) identifying from human antibody amino acid sequences a set of heavy and light chain surface exposed amino acid residues that is most closely identical to the set of surface exposed amino acid residues defined in step (c), wherein the heavy and light chain from the human antibody are or are not naturally paired;
(e) substituting, in the amino acid sequence of the non-human antibody to be humanized, the set of heavy and light chain surface exposed amino acid residues defined in step (c) with the set of heavy and light chain surface exposed amino acid residues identified in step (d);
(f) constructing a three-dimensional model of the variable region of the non-human antibody resulting from the substituting specified in step (e);
(g) identifying, by comparing the three-dimensional models constructed in steps (a) and (f), any amino acid residues from the sets identified in steps (c) or (d), that are within 5 Angstroms of any atom of any residue of the complementarity determining regions of the non-human antibodt to be humanized; and
(h) changing any residues identified in step (g) from the human to the original non-human amino acid residue to thereby define a non-human antibody humanizing set of surface exposed amino acid residues; with the proviso that step (a) need not be conducted first, but must be conducted prior to step (g).

Superhumanization

The method compares the non-human sequence with the functional human germline gene repertoire. Those human genes encoding canonical structures identical or closely related to the non-human sequences are selected. Those selected human genes with highest homology within the CDRs are chosen as FR donors. Finally, the non-human CDRs are grafted onto these human FRs. This method is described in patent WO 2005/079479 A2.

Human String Content Optimization

This method compares the non-human (e.g. mouse) sequence with the repertoire of human germline genes and the differences are scored as Human String Content (HSC) that quantifies a sequence at the level of potential MHC/T-cell epitopes. The target sequence is then humanized by maximizing its HSC rather than using a global identity measure to generate multiple diverse humanized variants (described in Molecular Immunology, 44, (2007) 1986-1998).

Framework Shuffling

The CDRs of the non-human antibody are fused in-frame to cDNA pools encompassing all known heavy and light chain human germline gene frameworks. Humanised antibodies are then selected by e.g. panning of the phage displayed antibody library. This is described in Methods 36, 43-60 (2005). Examples of cell binding agents include those agents described for use in WO2007/085930, which is incorporated herein.

Tumour-associate antigens and cognate antibodies for use in embodiments of the present invention are listed below.

Tumor-Associated Antigens and Cognate Antibodies

(1) BMPR1B (bone morphogenetic protein receptor-type IB)

Nucleotide

Genbank accession no. NM_001203

Genbank version no. NM_001203.2 GI:169790809

Genbank record update date: Sep. 23, 2012 02:06 PM

Polypeptide

Genbank accession no. NP_001194

Genbank version no. NP_001194.1 GI:4502431

Genbank record update date: Sep. 23, 2012 02:06 PM

Cross-References

ten Dijke,P., et al Science 264 (5155): 101-104 (1994), Oncogene 14 (11):1377-1382 (1997)); WO2004/063362 (claim 2); WO2003/042661 (claim 12);

US2003/134790-A1 (Page 38-39); WO2002/102235 (claim 13; Page 296); WO2003/055443 (Page 91-92); WO2002/99122 (Example 2; Page 528-530); WO2003/029421 (claim 6); WO2003/024392 (claim 2; FIG. 112); WO2002/98358 (claim 1; Page 183); WO2002/54940 (Page 100-101); WO2002/59377 (Page 349-350); WO2002/30268 (claim 27; Page 376); WO2001/48204 (Example; FIG. 4); NP_001194 bone morphogenetic protein receptor, type IB /pid=NP_001194.1.; MIM:603248; AY065994 (2) E16 (LAT1, SLC7A5)

Nucleotide

Genbank accession no. NM_003486

Genbank version no. NM_003486.5 GI:71979931

Genbank record update date: Jun. 27, 2012 12:06 PM

Polypeptide

Genbank accession no. NP_003477

Genbank version no. NP_003477.4 GI:71979932

Genbank record update date: Jun. 27, 2012 12:06 PM

Cross References

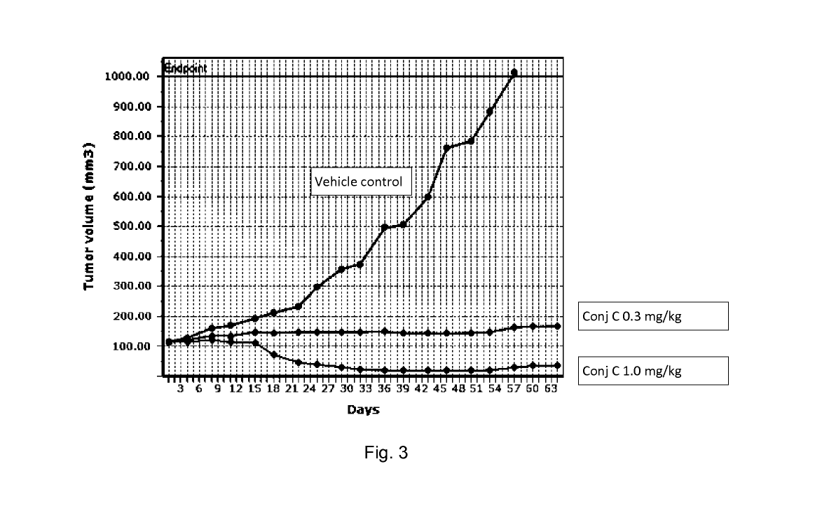

Biochem. Biophys. Res. Commun. 255 (2), 283-288 (1999), Nature 395 (6699):288-291 (1998), Gaugitsch, H. W., et al (1992) J.Biol. Chem. 267 (16):11267-11273); WO2004/048938 (Example 2); WO2004/032842 (Example IV); WO2003/042661 (claim 12); WO2003/016475 (claim 1); WO2002/78524 (Example 2); WO2002/99074 (claim 19; Page 127-129); WO2002/86443 (claim 27; Pages 222, 393); WO2003/003906 (claim 10; Page 293); WO2002/64798 (claim 33; Page 93-95); WO2000/14228 (claim 5; Page 133-136); US2003/224454 (FIG. 3); WO2003/025138 (claim 12; Page 150); NP_003477 solute carrier family 7 (cationic amino acid transporter, y+system), member 5 /pid=NP_003477.3-Homo sapiens; MIM:600182;; NM_015923.

(3) STEAP1 (six transmembrane epithelial antigen of prostate) Nucleotide

Genbank accession no. NM_012449

Genbank version no. NM_012449.2 GI:22027487

Genbank record update date: Sep. 9, 2012 02:57 PM

Polypeptide

Genbank accession no. NP_036581

Genbank version no. NP_036581.1 GI:9558759

Genbank record update date: Sep. 9, 2012 02:57 PM

Cross References

Cancer Res. 61 (15), 5857-5860 (2001), Hubert, R. S., et al (1999) Proc. Natl. Acad. Sci. U.S.A. 96 (25):14523-14528); WO2004/065577 (claim 6); WO2004/027049 (FIG. 14 EP1394274 (Example 11); WO2004/016225 (claim 2); WO2003/042661 (claim 12); US2003/157089 (Example 5); US2003/185830 (Example 5); US2003/064397 (FIG. 2); WO2002/89747 (Example 5; Page 618-619); WO2003/022995 (Example 9; FIG. 13A, Example 53; Page 173, Example 2; FIG. 2A); six transmembrane epithelial antigen of the prostate; MIM:604415.

(4) 0772P (CA125, MUC16)

Nucleotide

Genbank accession no. AF361486

Genbank version no. AF361486.3 GI:34501466

Genbank record update date: Mar. 11, 2010 07:56 AM

Polypeptide

Genbank accession no. AAK74120

Genbank version no. AAK74120.3 GI:34501467

Genbank record update date: Mar. 11, 2010 07:56 AM

Cross References J. Biol. Chem. 276 (29):27371-27375 (2001)); WO2004/045553 (claim 14); WO2002/92836 (claim 6; FIG. 12); WO2002/83866 (claim 15; Page 116-121); US2003/124140 (Example 16); GI:34501467;

(5) MPF (MPF, MSLN, SMR, megakaryocyte potentiating factor, mesothelin)

Nucleotide

Genbank accession no. NM_005823

Genbank version no. NM_005823.5 GI:293651528

Genbank record update date: Sep. 2, 2012 01:47 PM

Polypeptide

Genbank accession no. NP_005814

Genbank version no. NP_005814.2 GI:53988378

Genbank record update date: Sep. 2, 2012 01:47 PM

Cross References

Yamaguchi, N., et al Biol. Chem. 269 (2), 805-808 (1994), Proc. Natl. Acad. Sci. U.S.A. 96 (20):11531-11536 (1999), Proc. Natl. Acad. Sci. U.S.A. 93 (1):136-140 (1996), J.Biol. Chem. 270 (37):21984-21990 (1995)); WO2003/101283 (claim 14); (WO2002/102235 (claim 13; Page 287-288); WO2002/101075 (claim 4; Page 308-309); WO2002/71928 (Page 320-321); WO94/10312 (Page 52-57); IM:601051.

(6) Napi3b (NAPI-3B, NPTIIb, SLC34A2, solute carrier family 34 (sodium phosphate), member 2, type II sodium-dependent phosphate transporter 3b)

Nucleotide

Genbank accession no. NM_006424

Genbank version no. NM_006424.2 GI:110611905

Genbank record update date: Jul. 22, 2012 03:39 PM

Polypeptide

Genbank accession no. NP_006415

Genbank version no. NP_006415.2 GI:110611906

Genbank record update date: Jul. 22, 2012 03:39 PM

Cross References

J.Biol. Chem. 277 (22):19665-19672 (2002), Genomics 62 (2):281-284 (1999), Feild, J. A., et al (1999) Biochem. Biophys. Res. Commun. 258 (3):578-582); WO2004/022778 (claim 2); EP1394274 (Example 11); WO2002/102235 (claim 13; Page 326); EP0875569 (claim 1; Page 17-19); WO2001/57188 (claim 20; Page 329); WO2004/032842 (Example IV); WO2001/75177 (claim 24; Page 139-140); MIM:604217.

(7) Sema 5b (FLJ10372, KIAA1445, Mm.42015, SEMA58, SEMAG, Semaphorin 5b Hlog, sema domain, seven thrombospondin repeats (type 1 and type 1-like), transmembrane domain (TM) and short cytoplasmic domain, (semaphorin) 58)

Nucleotide

Genbank accession no. AB040878

Genbank version no. AB040878.1 GI:7959148

Genbank record update date: Aug. 2, 2006 05:40 PM

Polypeptide

Genbank accession no. BAA95969

Genbank version no. BAA95969.1 GI:7959149

Genbank record update date: Aug. 2, 2006 05:40 PM

Cross References

Nagase T., et al (2000) DNA Res. 7 (2):143-150); WO2004/000997 (claim 1); WO2003/003984 (claim 1); WO2002/06339 (claim 1; Page 50); WO2001/88133 (claim 1; Page 41-43, 48-58); WO2003/054152 (claim 20); WO2003/101400 (claim 11); Accession: Q9P283; Genew; HGNC:10737

(8) PSCA hlg (2700050C12Rik, C530008O16Rik, RIKEN cDNA 2700050C12, RIKEN cDNA 2700050C12 gene)

Nucleotide

Genbank accession no. AY358628

Genbank version no. AY358628.1 GI:37182377

Genbank record update date: Dec. 1, 2009 04:15 AM

Polypeptide

Genbank accession no. AAQ88991

Genbank version no. AAQ88991.1 GI:37182378

Genbank record update date: Dec. 1, 2009 04:15 AM

Cross References

Ross et al (2002) Cancer Res. 62:2546-2553; US2003/129192 (claim 2); US2004/044180 (claim 12); US2004/044179 (claim 11); US2003/096961 (claim 11); US2003/232056 (Example 5); WO2003/105758 16 (claim 12); US2003/206918 (Example 5); EP1347046 (claim 1); WO2003/025148 (claim 20); GI:37182378.

(9) ETBR (Endothelin type B receptor)

Nucleotide

Genbank accession no. AY275463

Genbank version no. AY275463.1 GI:30526094

Genbank record update date: Mar. 11, 2010 02:26 AM

Polypeptide

Genbank accession no. AAP32295

Genbank version no. AAP32295.1 GI:30526095

Genbank record update date: Mar. 11, 2010 02:26 AM

Cross References

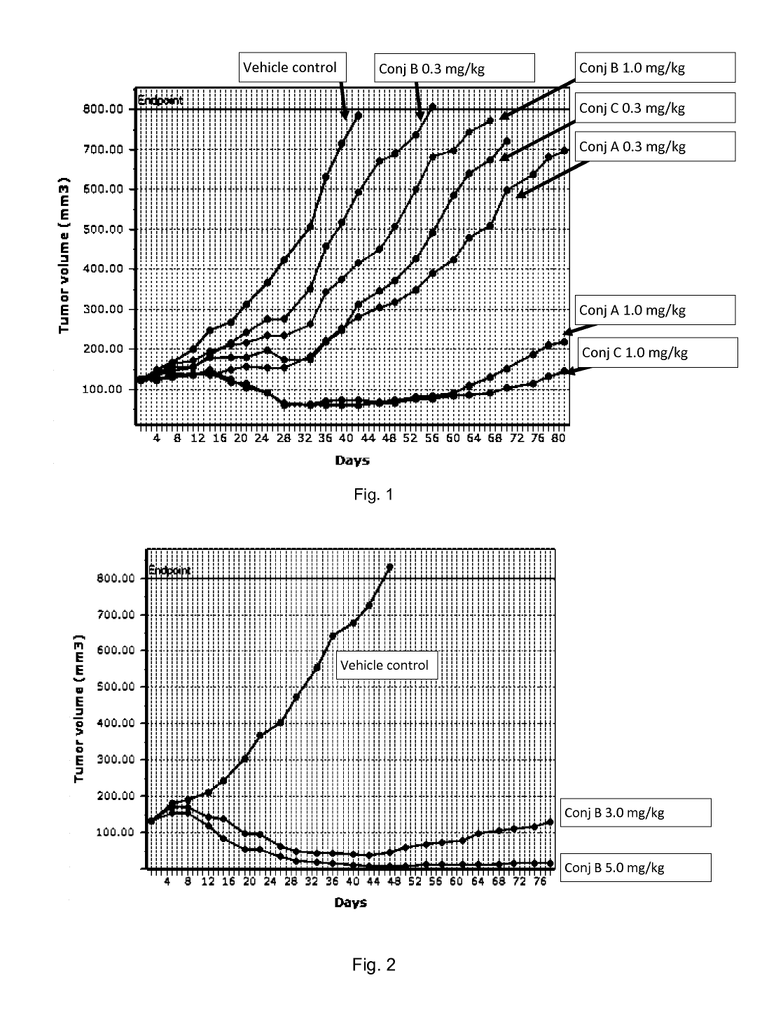

Nakamuta M., et al Biochem. Biophys. Res. Commun. 177, 34-39, 1991; Ogawa Y., et al Biochem. Biophys. Res. Commun. 178, 248-255, 1991; Arai H., et al Jpn. Circ. J.56, 1303-1307, 1992; Arai H., et al J.Biol. Chem. 268, 3463-3470, 1993; Sakamoto A., Yanagisawa M., et al Biochem. Biophys. Res. Commun. 178, 656-663, 1991; Elshourbagy N.A., et al J. Biol. Chem. 268, 3873-3879, 1993; Haendler B., et al J.Cardiovasc. Pharmacol. 20, sl-S4, 1992; Tsutsumi M., et al Gene 228, 43-49, 1999; Strausberg R. L., et al Proc. Natl. Acad. Sci. U.S.A. 99, 16899-16903, 2002; Bourgeois C., et al J. Clin. Endocrinol. Metab. 82, 3116-3123, 1997; Okamoto Y., et al Biol. Chem. 272, 21589-21596, 1997; Verheij J. B., et al Am. J. Med. Genet. 108, 223-225, 2002; Hofstra R. M. W., et al Eur. J. Hum. Genet. 5, 180-185, 1997; Puffenberger E. G., et al Cell 79, 1257-1266, 1994; Attie T., et al, Hum. Mol. Genet. 4, 2407-2409, 1995; Auricchio A., et al Hum. Mol. Genet. 5:351-354, 1996; Amiel J., et al Hum. Mol. Genet. 5, 355-357, 1996; Hofstra R. M. W., et al Nat. Genet. 12, 445-447, 1996; Svensson P. J., et al Hum. Genet. 103, 145-148, 1998; Fuchs S., et al Mol. Med. 7, 115-124, 2001; Pingault V., et al (2002) Hum. Genet. 111, 198-206; WO2004/045516 (claim 1); WO2004/048938 (Example 2); WO2004/040000 (claim 151); WO2003/087768 (claim 1); WO2003/016475 (claim 1); WO2003/016475 (claim 1); WO2002/61087 (FIG. 1); WO2003/016494 (FIG. 6); WO2003/025138 (claim 12; Page 144); WO2001/98351 (claim 1; Page 124-125); EP0522868 (claim 8; FIG. 2); WO2001/77172 (claim 1; Page 297-299); US2003/109676; U.S. Pat. No. 6,518,404 (FIG. 3); U.S. Pat. No. 5,773,223 (Claim 1a; Col 31-34); WO2004/001004.

(10) MSG783 (RNF124, hypothetical protein FLJ20315)

Nucleotide

Genbank accession no. NM_017763

Genbank version no. NM_017763.4 GI:167830482

Genbank record update date: Jul. 22, 2012 12:34 AM

Polypeptide

Genbank accession no. NP_060233

Genbank version no. NP_060233.3 GI:56711322

Genbank record update date: Jul. 22, 2012 12:34 AM

Cross References

WO2003/104275 (claim 1); WO2004/046342 (Example 2); WO2003/042661 (claim 12);

WO2003/083074 (claim 14; Page 61); WO2003/018621 (claim 1); WO2003/024392 (claim 2; FIG. 93); WO2001/66689 (Example 6); LocusID:54894.

(11) STEAP2 (HGNC_8639, IPCA-1, PCANAP1, STAMP1, STEAP2, STMP, prostate cancer

associated gene 1, prostate cancer associated protein 1, six transmembrane epithelial antigen of prostate 2, six transmembrane prostate protein)

Nucleotide

Genbank accession no. AF455138

Genbank version no. AF455138.1 GI:22655487

Genbank record update date: Mar. 11, 2010 01:54 AM

Polypeptide

Genbank accession no. AAN04080

Genbank version no. AAN04080.1 GI:22655488

Genbank record update date: Mar. 11, 2010 01:54 AM

Cross References

Lab. Invest. 82 (11):1573-1582 (2002)); WO2003/087306; US2003/064397 (claim 1; FIG. 1); WO2002/72596 (claim 13; Page 54-55); WO2001/72962 (claim 1; FIG. 4B);

WO2003/104270 (claim 11); WO2003/104270 (claim 16); US2004/005598 (claim 22); WO2003/042661 (claim 12); US2003/060612 (claim 12; FIG. 10); WO2002/26822 (claim 23; FIG. 2); WO2002/16429 (claim 12; FIG. 10); GI:22655488.

(12) TrpM4 (BR22450, FLJ20041, TRPM4, TRPM4B, transient receptor potential cation 5 channel, subfamily M, member 4)

Nucleotide

Genbank accession no. NM_017636

Genbank version no. NM_017636.3 GI:304766649

Genbank record update date: Jun. 29, 2012 11:27 AM

Polypeptide

Genbank accession no. NP_060106

Genbank version no. NP_060106.2 GI:21314671

Genbank record update date: Jun. 29, 2012 11:27 AM

Cross References

Xu, X.Z., et al Proc. Natl. Acad. Sci. U.S.A. 98 (19):10692-10697 (2001), Cell 109 (3):397-407 (2002), J.Biol. Chem. 278 (33):30813-30820 (2003)); US2003/143557 (claim 4); WO2000/40614 (claim 14; Page 100-103); WO2002/10382 (claim 1; FIG. 9A); WO2003/042661 (claim 12); WO2002/30268 (claim 27; Page 391); US2003/219806 (claim 4); WO2001/62794 (claim 14; FIG. 1A-D); MIM:606936.

(13) CRIPTO (CR, CR1, CRGF, CRIPTO, TDGF1, teratocarcinoma-derived growth factor)

Nucleotide

Genbank accession no. NM_003212

Genbank version no. NM_003212.3 GI:292494881

Genbank record update date: Sep. 23, 2012 02:27 PM

Polypeptide

Genbank accession no. NP_003203

Genbank version no. NP_003203.1 GI:4507425

Genbank record update date: Sep. 23, 2012 02:27 PM

Cross References

Ciccodicola, A., et al EMBO J.8 (7):1987-1991 (1989), Am. J.Hum. Genet. 49 (3):555-565 (1991)); US2003/224411 (claim 1); WO2003/083041 (Example 1); WO2003/034984 (claim 12); WO2002/88170 (claim 2; Page 52-53); WO2003/024392 (claim 2; FIG. 58); WO2002/16413 (claim 1; Page 94-95, 105); WO2002/22808 (claim 2; FIG. 1); U.S. Pat. No. 5,854,399 (Example 2; Col 17-18); U.S. Pat. No. 5,792,616 (FIG. 2); MIM:187395.

(14) CD21 (CR2 (Complement receptor 2) or C3DR (C3d/Epstein Barr virus receptor) or Hs. 73792)

Nucleotide

Genbank accession no M26004

Genbank version no. M26004.1 GI:181939

Genbank record update date: Jun. 23, 2010 08:47 AM

Polypeptide

Genbank accession no. AAA35786

Genbank version no. AAA35786.1 GI:181940

Genbank record update date: Jun. 23, 2010 08:47 AM

Cross References

Fujisaku et al (1989) J.Biol. Chem. 264 (4):2118-2125); Weis J. J., et al J.Exp. Med. 167, 1047-1066, 1988; Moore M., et al Proc. Natl. Acad. Sci. U.S.A. 84, 9194-9198, 1987; Barel M., et al Mol. Immunol. 35, 1025-1031, 1998; Weis J. J., et al Proc. Natl. Acad. Sci. U.S.A. 83, 5639-5643, 1986; Sinha S. K., et al (1993) J. Immunol. 150, 5311-5320; WO2004/045520 (Example 4); US2004/005538 (Example 1); WO2003/062401 (claim 9); WO2004/045520 (Example 4); WO91/02536 (FIG. 9.1-9.9); WO2004/020595 (claim 1); Accession: P20023; Q13866; Q14212; EMBL; M 26004; AAA35786.1.

(15) CD79b (CD79, βCD79β, IGb (immunoglobulin-associated beta), B29)

Nucleotide

Genbank accession no NM_000626

Genbank version no. NM_000626.2 GI:90193589

Genbank record update date: Jun. 26, 2012 01:53 PM

Polypeptide

Genbank accession no. NP_000617

Genbank version no. NP_000617.1 GI:11038674

Genbank record update date: Jun. 26, 2012 01:53 PM

Cross References

Proc. Natl. Acad. Sci. U.S.A. (2003) 100 (7):4126-4131, Blood (2002) 100 (9):3068-3076, Muller et al (1992) Eur. J. Immunol. 22 (6):1621-1625); WO2004/016225 (claim 2, FIG. 140); WO2003/087768, US2004/101874 (claim 1, page 102); WO2003/062401 (claim 9); WO2002/78524 (Example 2); US2002/150573 (claim 35 5, page 15); U.S. Pat. No. 5,644,033; WO2003/048202 (claim 1, pages 306 and 309); WO 99/58658, U.S. Pat. No. 6,534,482 (claim 13, FIG. 17A/B); WO2000/55351 (claim 11, pages 1145-1146); MIM:147245

(16) FcRH2 (IFGP4, IRTA4, SPAP1A (SH2 domain containing phosphatase anchor protein 1a), SPAP1B, SPAP1C)

Nucleotide

Genbank accession no NM_030764

Genbank version no. NM_030764.3 GI:227430280

Genbank record update date: Jun. 30, 2012 12:30 AM

Polypeptide

Genbank accession no. NP__110391

Genbank version no. NP__110391.2 GI:19923629

Genbank record update date: Jun. 30, 2012 12:30 AM

Cross References

AY358130); Genome Res. 13 (10):2265-2270 (2003),Immunogenetics 54 (2):87-95 (2002), Blood 99 (8):2662-2669 (2002), Proc. Natl. Acad. Sci. U.S.A. 98 (17)9772-9777 (2001), Xu, M. J., et al (2001) Biochem. Biophys. Res. Commun. 280 (3):768-775; WO2004/016225 (claim 2); WO2003/077836; WO2001/38490 (claim 5; FIG. 18D-1-18D-2); WO2003/097803 (claim 12); WO2003/089624 (claim 25);: MIM:606509.

(17) HER2 (ErbB2)

Nucleotide

Genbank accession no M11730

Genbank version no. M11730.1 GI:183986

Genbank record update date: Jun. 23, 2010 08:47 AM

Polypeptide

Genbank accession no. AAA75493

Genbank version no. AAA75493.1 GI:306840

Genbank record update date: Jun. 23, 2010 08:47 AM

Cross References

Coussens L., et al Science (1985) 230(4730):1132-1139); Yamamoto T., et al Nature 319, 230-234, 1986; Semba K., et al Proc. Natl. Acad. Sci. U.S.A. 82, 6497-6501, 1985; Swiercz J. M., et al J. Cell Biol. 165, 869-880, 2004; Kuhns J. J., et al J. Biol. Chem. 274, 36422-36427, 1999; Cho H.-S., et al Nature 421, 756-760, 2003; Ehsani A., et al (1993) Genomics 15, 426-429; WO2004/048938 (Example 2); WO2004/027049 (FIG. 11); WO2004/009622; WO2003/081210;WO2003/089904 (claim 9); WO2003/016475 (claim 1); US2003/118592; WO2003/008537 (claim 1); WO2003/055439 (claim 29; FIG. 1A-B); WO2003/025228 (claim 37; FIG. 5C); WO2002/22636 (Example 13; Page 95-107); WO2002/12341 (claim 68; FIG. 7); WO2002/13847 (Page 71-74); WO2002/14503 (Page 114-117); WO2001/53463 (claim 2; Page 41-46); WO2001/41787 (Page 15); WO2000/44899 (claim 52; FIG. 7); WO2000/20579 (claim 3; FIG. 2); U.S. Pat. No. 5,869,445 (claim 3; Col 31-38); WO9630514 (claim 2; Page 56-61); EP1439393 (claim 7); WO2004/043361 (claim 7); WO2004/022709; WO2001/00244 (Example 3; FIG. 4); Accession: P04626; EMBL; M11767; AAA35808.1. EMBL; M11761; AAA35808.1

ANTIBODIES

Abbott: US20110177095
For example, an antibody comprising CDRs having overall at least 80% sequence identity to CDRs having amino acid sequences of SEQ ID NO:3 (CDR-H1), SEQ ID NO:4 (CDR-H2), SEQ ID NO:5 (CDR-H3), SEQ ID NO:104 and/or SEQ ID NO:6 (CDR-L1), SEQ ID NO:7 (CDR-L2), and SEQ ID NO:8 (CDR-L3), wherein the anti-HER2 antibody or anti-HER2 binding fragment has reduced immunogenicity as compared to an antibody having a VH of SEQ ID NO:1 and a VL of SEQ ID NO:2.

Biogen: US20100119511
For example, ATCC accession numbers: PTA-10355, PTA-10356, PTA-10357, PTA-10358
For example, a purified antibody molecule that binds to HER2 comprising a all six CDR's from an antibody selected from the group consisting of BIIB71F10 (SEQ ID NOs:11, 13), BIIB69A09 (SEQ ID NOs:15, 17); BIIB67F10 (SEQ ID NOs:19, 21); BIIB67F11 (SEQ ID NOs:23, 25), BIIB66A12 (SEQ ID NOs:27, 29), BIIB66C01 (SEQ ID NOs:31, 33), BIIB65C10 (SEQ ID NOs:35, 37), BIIB65H09 (SEQ ID NOs:39, 41) and BIIB65B03 (SEQ ID NOs:43, 45), or CDRs which are identical or which have no more than two alterations from said CDRs.

Herceptin (Genentech)-U.S. Pat. No. 6,054,297; ATCC accession no. CRL-10463 (Genentech)

Pertuzumab (Genentech)
US20110117097
   for example, see SEQ IDs No. 15&16, SEQ IDs No. 17&18, SEQ IDs No. 23&24 & ATCC accession numbers HB-12215, HB-12216, CRL 10463, HB-12697.
US20090285837
US20090202546
   for example, ATCC accession numbers: HB-12215, HB-12216, CRL 10463, HB-12698.
US20060088523
   for example, ATCC accession numbers: HB-12215, HB-12216
   for example, an antibody comprising the variable light and variable heavy amino acid sequences in SEQ ID Nos. 3 and 4, respectively.
   for example, an antibody comprising a light chain amino acid sequence selected from SEQ ID No. 15 and 23, and a heavy chain amino acid sequence selected from SEQ ID No. 16 and 24
US20060018899
   for example, ATCC accession numbers: (7C2) HB-12215, (7F3) HB-12216, (4D5) CRL-10463, (2C4) HB-12697.
   for example, an antibody comprising the amino acid sequence in SEQ ID No. 23, or a deamidated and/or oxidized variant thereof.
US2011/0159014
   for example, an antibody having a light chain variable domain comprising the hypervariable regions of SEQ ID NO: 1″.
   for example, an antibody having a heavy chain variable domain comprising the hypervariable regions of SEQ ID NO: 2.
US20090187007

Glycotope: TrasGEX antibody http://www.glycotope.com/pipeline
For example, see International Joint Cancer Institute and Changhai Hospital Cancer Cent: HMTI-Fc Ab-Gao J., et al BMB Rep. 2009 Oct. 31; 42(10):636-41.

Symphogen: US20110217305

Union Stem Cell &Gene Engineering, China-Liu H Q., et al Xi Bao Yu Fen Zi Mian Yi Xue Za Zhi. 2010 May; 26(5):456-8.

(18) NCA (CEACAM6)

Nucleotide

Genbank accession no M18728

Genbank version no. M18728.1 GI:189084

Genbank record update date: Jun. 23, 2010 08:48 AM

Polypeptide

Genbank accession no. AAA59907

Genbank version no. AAA59907.1 GI:189085

Genbank record update date: Jun. 23, 2010 08:48 AM

Cross References

Barnett T., et al Genomics 3, 59-66, 1988; Tawaragi Y., et al Biochem. Biophys. Res. Commun. 150, 89-96, 1988; Strausberg R. L., et al Proc. Natl. Acad. Sci. U.S.A. 99:16899-16903, 2002; WO2004/063709; EP1439393 (claim 7); WO2004/044178 (Example 4); WO2004/031238; WO2003/042661 (claim 12); WO2002/78524 (Example 2); WO2002/86443 (claim 27; Page 427); WO2002/60317 (claim 2); Accession: P40199; Q14920; EMBL; M29541; AAA59915.1. EMBL; M18728.

(19) MDP (DPEP1)

Nucleotide

Genbank accession no BC017023

Genbank version no. BC017023.1 GI:16877538

Genbank record update date: Mar. 6, 2012 01:00 PM

Polypeptide

Genbank accession no. AAH17023

Genbank version no. AAH17023.1 GI:16877539

Genbank record update date: Mar. 6, 2012 01:00 PM

Cross References

Proc. Natl. Acad. Sci. U.S.A. 99 (26):16899-16903 (2002)); WO2003/016475 (claim 1); WO2002/64798 (claim 33; Page 85-87); JP05003790 (FIG. 6-8); WO99/46284 (FIG. 9); MIM:179780.

(20) IL20R-alpha (IL20Ra, ZCYTOR7)

Nucleotide

Genbank accession no AF184971

Genbank version no. AF184971.1 GI:6013324

Genbank record update date: Mar. 10, 2010 10:00 PM

Polypeptide

Genbank accession no. AAF01320

Genbank version no. AAF01320.1 GI:6013325

Genbank record update date: Mar. 10, 2010 10:00 PM

Cross References

Clark H. F., et al Genome Res. 13, 2265-2270, 2003; Mungall A. J., et al Nature 425, 805-811, 2003; Blumberg H., et al Cell 104, 9-19, 2001; Dumoutier L., et al J.Immunol. 167, 3545-3549, 2001; Parrish-Novak J., et al J.Biol. Chem. 277, 47517-47523, 2002; Pletnev S., et al (2003) Biochemistry 42:12617-12624; Sheikh F., et al (2004) J. Immunol. 172, 2006-2010; EP1394274 (Example 11); US2004/005320 (Example 5); WO2003/029262 (Page 74-75); WO2003/002717 (claim 2; Page 63); WO2002/22153 (Page 45-47); US2002/042366 (Page 20-21); WO2001/46261 (Page 57-59); WO2001/46232 (Page 63-65); WO98/37193 (claim 1; Page 55-59); Accession: Q9UHF4; Q6UWA9; Q96SH8; EMBL; AF184971; AAF01320.1.

(21) Brevican (BCAN, BEHAB)

Nucleotide

Genbank accession no AF229053

Genbank version no. AF229053.1 GI:10798902

Genbank record update date: Mar. 11, 2010 12:58 AM

Polypeptide

Genbank accession no. AAG23135

Genbank version no. AAG23135.1 GI:10798903

Genbank record update date: Mar. 11, 2010 12:58 AM

Cross References

Gary S. C., et al Gene 256, 139-147, 2000; Clark H. F., et al Genome Res. 13, 2265-2270, 2003; Strausberg R. L., et al Proc. Natl. Acad. Sci. U.S.A. 99, 16899-16903, 2002; US2003/186372 (claim 11); US2003/186373 (claim 11); US2003/119131 (claim 1; FIG. 52); US2003/119122 (claim 1; FIG. 52); US2003/119126 (claim 1); US2003/119121 (claim 1; FIG. 52); US2003/119129 (claim 1); US2003/119130 (claim 1); US2003/119128 (claim 1; FIG. 52); US2003/119125 (claim 1); WO2003/016475 (claim 1); WO2002/02634 (claim 1)

(22) EphB2R (DRT, ERK, Hek5, EPHT3, Tyro5)

Nucleotide

Genbank accession no NM_004442

Genbank version no. NM_004442.6 GI:111118979

Genbank record update date: Sep. 8, 2012 04:43 PM

Polypeptide

Genbank accession no. NP_004433

Genbank version no. NP_004433.2 GI:21396504

Genbank record update date: Sep. 8, 2012 04:43 PM

Cross References

Chan, J.and Watt, V. M., Oncogene 6 (6), 1057-1061 (1991) Oncogene 10 (5):897-905 (1995), Annu. Rev. Neurosci. 21:309-345 (1998), Int. Rev. Cytol. 196:177-244 (2000)); WO2003042661 (claim 12); WO200053216 (claim 1; Page 41); WO2004065576 (claim 1); WO2004020583 (claim 9); WO2003004529 (Page 128-132); WO200053216 (claim 1; Page 42); MIM:600997.

(23) ASLG659 (B7h)

Nucleotide

Genbank accession no. AX092328

Genbank version no. AX092328.1 GI:13444478

Genbank record update date: Jan. 26, 2011 07:37 AM

Cross References

US2004/0101899 (claim 2); WO2003104399 (claim 11); WO2004000221 (FIG. 3); US2003/165504 (claim 1); US2003/124140 (Example 2); US2003/065143 (FIG. 60); WO2002/102235 (claim 13; Page 299); US2003/091580 (Example 2); WO2002/10187 (claim 6; FIG. 10); WO2001/94641 (claim 12; FIG. 7b); WO2002/02624 (claim 13; FIG. 1A-1B); US2002/034749 (claim 54; Page 45-46); WO2002/06317 (Example 2; Page 320-321, claim 34; Page 321-322); WO2002/71928 (Page 468-469); WO2002/02587 (Example 1; FIG. 1); WO2001/40269 (Example 3; Pages 190-192); WO2000/36107 (Example 2; Page 205-207); WO2004/053079 (claim 12); WO2003/004989 (claim 1); WO2002/71928 (Page 233-234, 452-453); WO 01/16318.

(24) PSCA (Prostate stem cell antigen precursor)

Nucleotide

Genbank accession no AJ297436

Genbank version no. AJ297436.1 GI:9367211

Genbank record update date: Feb. 1, 2011 11:25 AM

Polypeptide

Genbank accession no. CAB97347

Genbank version no. CAB97347.1 GI:9367212

Genbank record update date: Feb. 1, 2011 11:25 AM

Cross References

Reiter R. E., et al Proc. Natl. Acad. Sci. U.S.A. 95, 1735-1740, 1998; Gu Z., et al Oncogene 19, 1288-1296, 2000; Biochem. Biophys. Res. Commun. (2000) 275(3):783-788; WO2004/022709; EP1394274 (Example 11); US2004/018553 (claim 17); WO2003/008537 (claim 1); WO2002/81646 (claim 1; Page 164); WO2003/003906 (claim 10; Page 288); WO2001/40309 (Example 1; FIG. 17); US2001/055751 (Example 1; FIG. 1b); WO2000/32752 (claim 18; FIG. 1); WO98/51805 (claim 17; Page 97); WO98/51824 (claim 10; Page 94); WO98/40403 (claim 2; FIG. 1B); Accession: 043653; EMBL; AF043498; AAC39607.1

(25) GEDA

Nucleotide

Genbank accession no AY260763

Genbank version no. AY260763.1 GI:30102448

Genbank record update date: Mar. 11, 2010 02:24 AM

Polypeptide

Genbank accession no. AAP14954

Genbank version no. AAP14954.1 GI:30102449

Genbank record update date: Mar. 11, 2010 02:24 AM

Cross References

AP14954 lipoma HMGIC fusion-partnerlike protein/pid=AAP14954.1 -Homo sapiens (human); WO2003/054152 (claim 20); WO2003/000842 (claim 1); WO2003/023013 (Example 3, claim 20); US2003/194704 (claim 45); GI:30102449;

(26) BAFF-R (B cell-activating factor receptor, BLyS receptor 3, BR3)

Nucleotide

Genbank accession no AF116456

Genbank version no. AF116456.1 GI:4585274

Genbank record update date: Mar. 10, 2010 09:44 PM

Polypeptide

Genbank accession no. AAD25356

Genbank version no. AAD25356.1 GI:4585275

Genbank record update date: Mar. 10, 2010 09:44 PM

Cross References

BAFF receptor/pid=NP_443177.1-Homo sapiens: Thompson, J. S., et al Science 293 (5537), 2108-2111 (2001); WO2004/058309; WO2004/011611; WO2003/045422 (Example; Page 32-33); WO2003/014294 (claim 35; FIG. 6B); WO2003/035846 (claim 70; Page 615-616); WO2002/94852 (Col 136-137); WO2002/38766 (claim 3; Page 133); WO2002/24909 (Example 3; FIG. 3); MIM:606269; NP_443177.1; NM_052945_1; AF132600

(27) CD22 (B-cell receptor CD22-B isoform, BL-CAM, Lyb-8, Lyb8, SIGLEC-2, FLJ22814)

Nucleotide

Genbank accession no AK026467

Genbank version no. AK026467.1 GI:10439337

Genbank record update date: Sep. 11, 2006 11:24 PM

Polypeptide

Genbank accession no. BAB15489

Genbank version no. BAB15489.1 GI:10439338

Genbank record update date: Sep. 11, 2006 11:24 PM

Cross References

Wilson et al (1991) J. Exp. Med. 173:137-146; WO2003/072036 (claim 1; FIG. 1); IM:107266; NP_001762.1; NM_001771_1.

(27a) CD22 (CD22 molecule)

Nucleotide

Genbank accession no X52785

Genbank version no. X52785.1 GI:29778

Genbank record update date: Feb. 2, 2011 10:09 AM

Polypeptide

Genbank accession no. CAA36988

Genbank version no. CAA36988.1 GI:29779

Genbank record update date: Feb. 2, 2011 10:09 AM

Cross References

Stamenkovic I. et al., Nature 345 (6270), 74-77 (1990)??

Other information

Official Symbol: CD22

Other Aliases: SIGLEC-2, SIGLEC2

Other Designations: B-cell receptor CD22; B-lymphocyte cell adhesion molecule; BL-CAM; CD22 antigen; T-cell surface antigen Leu-14; sialic acid binding Ig-like lectin 2; sialic acid-binding Ig-like lectin 2

ANTIBODIES

G5/44 (Inotuzumab): DiJoseph J F.,et al Cancer Immunol Immunother. 2005 Jan.; 54(1):11-24.

Epratuzumab-Goldenberg D M., et al Expert Rev Anticancer Ther. 6(10): 1341-53, 2006.

(28) CD79a (CD79A, CD79alpha), immunoglobulin-associated alpha, a B cell-specific protein that covalently interacts with Ig beta (CD79B) and forms a complex on the surface with Ig M molecules, transduces a signal involved in B-cell differentiation), pl: 4.84, MW: 25028 TM: 2 [P] Gene Chromosome: 19q13.2).

Nucleotide

Genbank accession no NM_001783

Genbank version no. NM_001783.3 GI:90193587

Genbank record update date: Jun. 26, 2012 01:48 PM

Polypeptide

Genbank accession no. NP_001774

Genbank version no. NP_001774.1 GI:4502685

Genbank record update date: Jun. 26, 2012 01:48 PM

Cross References

WO2003/088808, US2003/0228319; WO2003/062401 (claim 9); US2002/150573 (claim 4, pages 13-14); WO99/58658 (claim 13, FIG. 16); WO92/07574 (FIG. 1); US5644033; Ha et al (1992) J. Immunol. 148(5):1526-1531; Muller et al (1992) Eur. J. Immunol. 22:1621-1625; Hashimoto et al (1994) Immunogenetics 40(4):287-295; Preud'homme et al (1992) Clin. Exp. Immunol. 90(1):141-146; Yu et al (1992) J. Immunol. 148(2) 633-637; Sakaguchi et al (1988) EMBO J.7(11):3457-3464

(29) CXCRS (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pl: 8.54 MW: 41959 TM: 7 [P]Gene Chromosome: 11q23.3,

Nucleotide

Genbank accession no NM_001716

Genbank version no. NM_001716.4 GI:342307092

Genbank record update date: Sep. 30, 2012 01:49 PM

Polypeptide

Genbank accession no. NP_001707

Genbank version no. NP_001707.1 GI:4502415

Genbank record update date: Sep. 30, 2012 01:49 PM

Cross References

WO2004/040000; WO2004/015426; US2003/105292 (Example 2); U.S. Pat. No. 6,555,339 (Example 2); WO2002/61087 (FIG. 1); WO2001/57188 (claim 20, page 269); WO2001/72830 (pages 12-13); WO2000/22129 (Example 1, pages 152-153, Example 2, pages 254-256); WO99/28468 (claim 1, page 38); U.S. Pat. No. 5,440,021 (Example 2, col 49-52); WO94/28931 (pages 56-58); WO92/17497 (claim 7, FIG. 5); Dobner et al (1992) Eur. J. Immunol. 22:2795-2799; Barella et al (1995) Biochem. J.309:773-779

(30) HLA-DOB (Beta subunit of MHC class II molecule (la antigen) that binds peptides and presents them to CD4+T lymphocytes); 273 aa, pl: 6.56, MW: 30820. TM: 1 [P] Gene Chromosome: 6p21.3)

Nucleotide

Genbank accession no NM_002120

Genbank version no. NM_002120.3 GI:118402587

Genbank record update date: Sep. 8, 2012 04:46 PM

Polypeptide

Genbank accession no. NP_002111

Genbank version no. NP_002111.1 GI:4504403

Genbank record update date: Sep. 8, 2012 04:46 PM

Cross References

Tonnelle et al (1985) EMBO J.4(11):2839-2847; Jonsson et al (1989) Immunogenetics 29(6):411-413; Beck et al (1992) J. Mol. Biol. 228:433-441; Strausberg et al (2002) Proc. Natl. Acad. Sci USA 99:16899-16903; Servenius et al (1987) J. Biol. Chem. 262:8759-8766; Beck et al (1996) J. Mol. Biol. 255:1-13; Naruse et al (2002) Tissue Antigens 59:512-519; WO99/58658 (claim 13, FIG. 15); US6153408 (Col 35-38); US5976551 (col 168-170); US6011146 (col 145-146); Kasahara et al (1989) Immunogenetics 30(1):66-68; Larhammar et al (1985) J. Biol. Chem. 260(26):14111-14119

(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pl: 7.63, MW: 47206 TM: 1 [P] Gene Chromosome: 17p13.3).

Nucleotide

Genbank accession no NM_002561

Genbank version no. NM_002561.3 GI:325197202

Genbank record update date: Jun. 27, 2012 12:41 AM

Polypeptide

Genbank accession no. NP_002552

Genbank version no. NP_002552.2 GI:28416933

Genbank record update date: Jun. 27, 2012 12:41 AM

Cross References

Le et al (1997) FEBS Lett. 418(1-2):195-199; WO2004/047749; WO2003/072035 (claim 10); Touchman et al (2000) Genome Res. 10:165-173; WO2002/22660 (claim 20); WO2003/093444 (claim 1); WO2003/087768 (claim 1); WO2003/029277 (page 82) (32) CD72 (B-cell differentiation antigen CD72, Lyb-2); 359 aa, pl: 8.66, MW: 40225, TM: 1 5 [P] Gene Chromosome: 9p13.3).

Nucleotide

Genbank accession no NM_001782

Genbank version no. NM_001782.2 GI:194018444

Genbank record update date: Jun. 26, 2012 01:43 PM

Polypeptide

Genbank accession no. NP_001773

Genbank version no. NP_001773.1 GI:4502683

Genbank record update date: Jun. 26, 2012 01:43 PM

Cross References

WO2004042346 (claim 65); WO2003/026493 (pages 51-52, 57-58); WO2000/75655 (pages 105-106); Von Hoegen et al (1990) J. Immunol. 144 (12):4870-4877; Strausberg et al (2002) Proc. Natl. Acad. Sci USA 99:16899-16903.

(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis); 661 aa, pl: 6.20, MW: 74147 TM: 1 [P] Gene Chromosome: 5q12).

Nucleotide

Genbank accession no NM_005582

Genbank version no. NM_005582.2 GI:167555126

Genbank record update date: Sep. 2, 2012 01:50 PM

Polypeptide

Genbank accession no. NP_005573

Genbank version no. NP_005573.2 GI:167555127

Genbank record update date: Sep. 2, 2012 01:50 PM

Cross References US2002/193567; WO97/07198 (claim 11, pages 39-42); Miura et al (1996) Genomics 38(3):299-304; Miura et al (1998) Blood 92:2815-2822; WO2003/083047; WO97/44452 (claim 8, pages 57-61); WO2000/12130 (pages 24-26).

(34) FcRH1 (Fc receptor-like protein 1, a putative receptor for the immunoglobulin Fc domain that contains C2 type Ig-like and ITAM domains, may have a role in B-lymphocyte differentiation); 429 aa, pl: 5.28, MW: 46925 TM: 1 [P] Gene Chromosome: 1q21-1q22)

Nucleotide

Genbank accession no NM_052938

Genbank version no. NM_052938.4 GI:226958543

Genbank record update date: Sep. 2, 2012 01:43 PM

Polypeptide

Genbank accession no. NP_443170

Genbank version no. NP_443170.1 GI:16418419

Genbank record update date: Sep. 2, 2012 01:43 PM

Cross References

WO2003/077836; WO2001/38490 (claim 6, FIG. 18E-1-18-E-2); Davis et al (2001) Proc. Natl. Acad. Sci USA 98(17):9772-9777; WO2003/089624 (claim 8); EP1347046 (claim 1); WO2003/089624 (claim 7).

(35) IRTA2 (Immunoglobulin superfamily receptor translocation associated 2, a putative immunoreceptor with possible roles in B cell development and lymphoma genesis; deregulation of the gene by translocation occurs in some B cell malignancies); 977 aa, pl: 6.88, MW: 106468, TM: 1 [P] Gene Chromosome: 1q21)

Nucleotide

Genbank accession no AF343662

Genbank version no. AF343662.1 GI:13591709

Genbank record update date: Mar. 11, 2010 01:16 AM

Polypeptide

Genbank accession no. AAK31325

Genbank version no. AAK31325.1 GI:13591710

Genbank record update date: Mar. 11, 2010 01:16 AM

Cross References

AF343663, AF343664, AF343665, AF369794, AF397453, AK090423, AK090475, AL834187, AY358085; Mouse:AK089756, AY158090, AY506558; NP_112571.1; WO2003/024392 (claim 2, FIG. 97); Nakayama et al (2000) Biochem. Biophys. Res. Commun. 277(1):124-127; WO2003/077836; WO2001/38490 (claim 3, FIG. 18B-1-18B-2).

(36) TENB2 (TMEFF2, tomoregulin, TPEF, HPP1, TR, putative transmembrane proteoglycan, related to the EGF/heregulin family of growth factors and follistatin); 374 aa)

Nucleotide

Genbank accession no AF179274

Genbank version no. AF179274.2 GI:12280939

Genbank record update date: Mar. 11, 2010 01:05 AM

Polypeptide

Genbank accession no. AAD55776

Genbank version no. AAD55776.2 GI:12280940

Genbank record update date: Mar. 11, 2010 01:05 AM

Cross References

NCBI Accession: AAD55776, AAF91397, AAG49451, NCBI RefSeq: NP_057276; NCBI

Gene: 23671; OMIM: 605734; SwissProt Q9UIK5; AY358907, CAF85723, CQ782436; WO2004/074320; JP2004113151; WO2003/042661; WO2003/009814; EP1295944 (pages 69-70); WO2002/30268 (page 329); WO2001/90304; US2004/249130; US2004/022727; WO2004/063355; US2004/197325; US2003/232350; US2004/005563; US2003/124579; Horie et al (2000) Genomics 67:146-152; Uchida et al (1999) Biochem. Biophys. Res. Commun. 266:593-602; Liang et al (2000) Cancer Res. 60:4907-12; Glynne-Jones et al (2001) Int J Cancer. Oct. 15; 94(2):178-84.

(37) PSMA—FOLH1 (Folate hydrolase (prostate-specific membrane antigen) 1)

Nucleotide

Genbank accession no M99487

Genbank version no. M99487.1 GI:190663

Genbank record update date: Jun. 23, 2010 08:48 AM

Polypeptide

Genbank accession no. AAA60209

Genbank version no. AAA60209.1 GI:190664

Genbank record update date: Jun. 23, 2010 08:48 AM

Cross References

Israeli R.S., et al Cancer Res. 53 (2), 227-230 (1993)

Other information

Official Symbol: FOLH1

Other Aliases: GIG27, FGCP, FOLH, GCP2, GCPII, NAALAD1, NAALAdase, PSM, PSMA, mGCP

Other Designations: N-acetylated alpha-linked acidic dipeptidase 1; N-acetylated-alpha-linked acidic dipeptidase I; NAALADase I; cell growth-inhibiting gene 27 protein; folylpoly-gamma-glutamate carboxypeptidase; glutamate carboxylase II; glutamate carboxypeptidase 2; glutamate carboxypeptidase II; membrane glutamate carboxypeptidase; prostate specific membrane antigen variant F; pteroylpoly-gamma-glutamate carboxypeptidase

ANTIBODIES

U.S. Pat. No. 7,666,425:

Antibodies produces by Hybridomas having the following ATCC references:ATCC accession No. HB-12101, ATCC accession No. HB-12109, ATCC accession No. HB-12127 and ATCC accession No. HB-12126.

Proscan: a monoclonal antibody selected from the group consisting of 8H12, 3E11, 17G1, 2964, 30C1 and 20F2 (U.S. Pat. No. 7,811,564; Moffett S., et al Hybridoma (Larchmt). 2007 Dec.; 26(6):363-72).

Cytogen: monoclonal antibodies 7E11-05 (ATCC accession No. HB 10494) and 9H10-A4

(ATCC accession No. H611430)-U.S. Pat. No. 5,763,202

GlycoMimetics: NUH2 -ATCC accession No. HB 9762 (U.S. Pat. No. 7,135,301)

Human Genome Science: HPRAJ70 - ATCC accession No. 97131 (U.S. Pat. No. 6,824,993); Amino acid sequence encoded by the cDNA clone (HPRAJ70) deposited as American Type Culture Collection (“ATCC”) Deposit No. 97131

Medarex: Anti-PSMA antibodies that lack fucosyl residues-U.S. Pat. No. 7,875,278

Mouse anti-PSMA antibodies include the 3F5.4G6, 3D7.1.1, 4E10-1.14, 3E11, 4D8, 3E6, 3C9, 2C7, 1G3, 3C4, 3C6, 4D4, 1G9, 5C8B9, 3G6, 4C8B9, and monoclonal antibodies. Hybridomas secreting 3F5.4G6, 3D7.1.1, 4E10-1.14, 3E11, 4D8, 3E6, 3C9, 2C7, 1G3, 3C4, 3C6, 4D4, 1G9, 5C8B9, 3G6 or 4C8B9 have been publicly deposited and are described in U.S. Pat. No. 6,159,508. Relevant hybridomas have been publicly deposited and are described in U.S. Pat. No. 6,107,090. Moreover, humanized anti-PSMA antibodies, including a humanized version of J591, are described in further detail in PCT Publication WO 02/098897.

Other mouse anti-human PSMA antibodies have been described in the art, such as mAb 107-1A4 (Wang, S. et al. (2001) Int. J. Cancer 92:871-876) and mAb 2C9 (Kato, K. et al. (2003) Int. J. Urol. 10:439-444).

Examples of human anti-PSMA monoclonal antibodies include the 4A3, 7F12, 8C12, 8A11, 16F9, 2A10, 2C6, 2F5 and 1C3 antibodies, isolated and structurally characterized as originally described in PCT Publications WO 01/09192 and WO 03/064606 and in U.S. Provisional Application Ser. No. 60/654,125, entitled “Human Monoclonal Antibodies to Prostate Specific Membrane Antigen (PSMA)”, filed on Feb. 18, 2005. The V.sub.H amino acid sequences of 4A3, 7F12, 8C12, 8A11, 16F9, 2A10, 2C6, 2F5 and 1C3 are shown in SEQ ID NOs: 1-9, respectively. The V.sub.L amino acid sequences of 4A3, 7F12, 8C12, 8A11, 16F9, 2A10, 2C6, 2F5 and 1C3 are shown in SEQ ID NOs: 10-18, respectively.

Other human anti-PSMA antibodies include the antibodies disclosed in PCT Publication WO 03/034903 and US Application No. 2004/0033229.

NW Biotherapeutics: A hybridoma cell line selected from the group consisting of 3F5.4G6 having ATCC accession number HB12060, 3D7-1.I. having ATCC accession number HB12309, 4E10-1.14 having ATCC accession number HB12310, 3E11 (ATCC HB12488), 4D8 (ATCC HB12487), 3E6 (ATCC HB12486), 3C9 (ATCC HB12484), 2C7 (ATCC HB12490), 1G3 (ATCC HB12489), 3C4 (ATCC HB12494), 3C6 (ATCC HB12491), 4D4 (ATCC HB12493), 1G9 (ATCC HB12495), 5C8B9 (ATCC HB12492) and 3G6 (ATCC HB12485)—see U.S. Pat. No. 6,150,508

PSMA Development Company/Progenics/Cytogen—Seattle Genetics: mAb 3.9, produced by the hybridoma deposited under ATCC Accession No. PTA-3258 or mAb 10.3, produced by the hybridoma deposited under ATCC Accession No. PTA-3347 -U.S. Pat. No. 7,850,971

PSMA Development Company—Compositions of PSMA antibodies (US 20080286284, Table 1)
This application is a divisional of U.S. patent application Ser. No. 10/395,894, filed on Mar. 21, 2003 (U.S. Pat. No. 7,850,971)

University Hospital Freiburg, Germany—mAbs 3/A12, 3/E7, and 3/F11 (Wolf P., et al Prostate. 2010 Apr. 1; 70(5):562-9).

(38) SST (Somatostatin Receptor; note that there are 5 subtypes)

(38.1) SSTR2 (Somatostatin receptor 2)

Nucleotide

Genbank accession no NM_001050

Genbank version no. NM_001050.2 GI:44890054

Genbank record update date: Aug. 19, 2012 01:37 PM

Polypeptide

Genbank accession no. NP_001041

Genbank version no. NP_001041.1 GI:4557859

Genbank record update date: Aug. 19, 2012 01:37 PM

Cross References

Yamada Y., et al Proc. Natl. Acad. Sci. U.S.A. 89 (1), 251-255 (1992); Susini C., et al Ann Oncol. 2006 Dec.; 17(12):1733-42

Other Information

Official Symbol: SSTR2

Other Designations: SRIF-1; SS2R; somatostatin receptor type 2

(38.2) SSTR5 (Somatostatin receptor 5)

Nucleotide

Genbank accession no D16827

Genbank version no. D16827.1 GI:487683

Genbank record update date: Aug. 1, 2006 12:45 PM

Polypeptide

Genbank accession no. BAA04107

Genbank version no. BAA04107.1 GI:487684

Genbank record update date: Aug. 1, 2006 12:45 PM

Cross References

Yamada,Y., et al Biochem. Biophys. Res. Commun. 195 (2), 844-852 (1993)

Other information

Official Symbol: SSTR5

Other Aliases: SS-5-R

Other Designations: Somatostatin receptor subtype 5; somatostatin receptor type 5

(38.3) SSTR1

(38.4)SSTR3

(38.5) SSTR4

AvB6 -Both Subunits (39+40)

(39) ITGAV (Integrin, alpha V;

Nucleotide

Genbank accession no M14648 J02826 M18365

Genbank version no. M14648.1 GI:340306

Genbank record update date: Jun. 23, 2010 08:56 AM

Polypeptide

Genbank accession no. AAA36808

Genbank version no. AAA36808.1 GI:340307

Genbank record update date: Jun. 23, 2010 08:56 AM

Cross References

Suzuki S., et al Proc. Natl. Acad. Sci. U.S.A. 83 (22), 8614-8618 (1986)

Other information

Official Symbol: ITGAV

Other Aliases: CD51, MSK8, VNRA, VTNR

Other Designations: antigen identified by monoclonal antibody L230; integrin alpha-V; integrin alphaVbeta3; integrin, alpha V (vitronectin receptor, alpha polypeptide, antigen CD51); vitronectin receptor subunit alpha (40) ITGB6 (Integrin, beta 6)

Nucleotide

Genbank accession no NM_000888

Genbank version no. NM_000888.3 GI:9966771

Genbank record update date: Jun. 27, 2012 12:46 AM

Polypeptide

Genbank accession no. NP_000879

Genbank version no. NP_000879.2 GI:9625002

Genbank record update date: Jun. 27, 2012 12:46 AM

Cross References

Sheppard D. J., et al Biol. Chem. 265 (20), 11502-11507 (1990)

Other Information

Official Symbol: ITGB6

Other Designations: integrin beta-6

ANTIBODIES

Biogen: U.S. Pat. No. 7,943,742 —Hybridoma clones 6.3G9 and 6.8G6 were deposited with the ATCC, accession numbers ATCC PTA−3649 and −3645, respectively.

Biogen: U.S. Pat. No. 7,465,449—In some embodiments, the antibody comprises the same heavy and light chain polypeptide sequences as an antibody produced by hybridoma 6.1A8, 6.3G9, 6.8G6, 6.261, 6.2610, 6.2A1, 6.2E5, 7.1G10, 7.7G5, or 7.105.

Centocor (J&J): U.S. Pat. No. 7,550,142; U.S. Pat. No. 7,163,681

For example in U.S. 7,550,142 —an antibody having human heavy chain and human light chain variable regions comprising the amino acid sequences shown in SEQ ID NO: 7 and SEQ ID NO: 8.

Seattle Genetics: 15H3 (Ryan M C., et al Cancer Res Apr. 15, 2012; 72(8 Supplement): 4630)

(41) CEACAM5 (Carcinoembryonic antigen-related cell adhesion molecule 5)

Nucleotide

Genbank accession no M17303

Genbank version no. M17303.1 GI:178676

Genbank record update date: Jun. 23, 2010 08:47 AM

Polypeptide

Genbank accession no. AA659513

Genbank version no. AA659513.1 GI:178677

Genbank record update date: Jun. 23, 2010 08:47 AM

Cross References

Beauchemin N., et al Mol. Cell. Biol. 7 (9), 3221-3230 (1987)

Other Information

Official Symbol: CEACAM5

Other Aliases: CD66e, CEA

Other Designations: meconium antigen 100

ANTIBODIES

AstraZeneca-Medlmmune:US 20100330103; US20080057063;
US20020142359
   for example an antibody having complementarity determining regions (CDRs) with the following sequences: heavy chain; CDR1-DNYMH, CDR2-WIDPENGDTE YAPKFRG, CDR3-LIYAGYLAMD Y; and light chain CDR1- SASSSVTYMH, CDR2-STSNLAS, CDR3-QQRSTYPLT.
   Hybridoma 806.077 deposited as European Collection of Cell Cultures (ECACC) deposit no. 96022936.

Research Corporation Technologies, Inc.:U.S. Pat. No. 5,047,507

Bayer Corporation: U.S. Pat. No. 6,013,772

BioAlliance: U.S. Pat. No. 7,982,017; U.S. Pat. No. 7,674,605
U.S. Pat. No. 7,674,605
   an antibody comprising the heavy chain variable region sequence from the amino acid sequence of SEQ ID NO: 1, and the light chain variable region sequence from the amino acid sequence of SEQ ID NO:2.
   an antibody comprising the heavy chain variable region sequence from the amino acid sequence of SEQ ID NO:5, and the light chain variable region sequence from the amino acid sequence of SEQ ID NO:6.

Celltech Therapeutics Limited: U.S. Pat. No. 5,877,293

The Dow Chemical Company: U.S. Pat. No. 5,472,693; U.S. Pat. No. 6,417,337; U.S. Pat. No. 6,333,405
U.S. Pat. No. 5,472,693—for example, ATCC No. CRL-11215
U.S. Pat. No. 6,417,337—for example, ATCC CRL-12208
U.S. Pat. No. 6,333,405—for example, ATCC CRL-12208

Immunomedics, Inc: U.S. Pat. No. 7,534,431; U.S. Pat. No. 7,230,084; U.S. Pat. No. 7,300,644; U.S. Pat. No. 6,730,300;
U.S. Pat. No. 20110189085
   an antibody having CDRs of the light chain variable region comprise: CDR1 comprises KASQDVGTSVA (SEQ ID NO: 20); CDR2 comprises WTSTRHT (SEQ ID NO: 21); and CDR3 comprises QQYSLYRS (SEQ ID NO: 22); and the CDRs of the heavy chain variable region of said anti-CEA antibody comprise: CDR1 comprises TYWMS (SEQ ID NO: 23); CDR2 comprises EIHPDSSTINYAPSLKD (SEQ ID NO: 24); and CDR3 comprises LYFGFPWFAY (SEQ ID NO: 25).
US20100221175; US20090092598; US20070202044; US20110064653;
US20090185974; US20080069775.

(42) MET (met proto-oncogene; hepatocyte growth factor receptor)

Nucleotide

Genbank accession no M35073

Genbank version no. M35073.1 GI:187553

Genbank record update date: Mar. 6, 2012 11:12 AM

Polypeptide

Genbank accession no. AAA59589

Genbank version no. AAA59589.1 GI:553531

Genbank record update date: Mar. 6, 2012 11:12 AM

Cross References

Dean M., et al Nature 318 (6044), 385-388 (1985)

Other Information

Official Symbol: MET

Other Aliases: AUTS9, HGFR, RCCP2, c-Met

Other Designations: HGF receptor; HGF/SF receptor; SF receptor; hepatocyte growth factor receptor; met proto-oncogene tyrosine kinase; proto-oncogene c-Met; scatter factor receptor; tyrosine-protein kinase Met

ANTIBODIES

Abgenix/Pfizer: US20100040629
for example, the antibody produced by hybridoma 13.3.2 having American Type Culture Collection (ATCC) accession number PTA-5026; the antibody produced by hybridoma 9.1.2 having ATCC accession number PTA-5027; the antibody produced by hybridoma 8.70.2 having ATCC accession number PTA-5028; or the antibody produced by hybridoma 6.90.3 having ATCC accession number PTA-5029.

Amgen/Pfizer: US20050054019
for example, an antibody comprising a heavy chain having the amino acid sequences set forth in SEQ ID NO: 2 where X2 is glutamate and X4 is serine and a light chain having the amino acid sequence set forth in SEQ ID NO: 4 where X8 is alanine, without the signal sequences; an antibody comprising a heavy chain having the amino acid sequences set forth in SEQ ID NO: 6 and a light chain having the amino acid sequence set forth in SEQ ID NO: 8, without the signal sequences; an antibody comprising a heavy chain having the amino acid sequences set forth in SEQ ID NO: 10 and a light chain having the amino acid sequence set forth in SEQ ID NO: 12, without the signal sequences; or an antibody comprising a heavy chain having the amino acid sequences set forth in SEQ ID NO: 14 and a light chain having the amino acid sequence set forth in SEQ ID NO: 16, without the signal sequences.

Agouron Pharmaceuticals (Now Pfizer): US20060035907

Eli Lilly: US20100129369

Genentech: U.S. Pat. No. 5,686,292; US20100028337; US20100016241; US20070129301; US20070098707; US20070092520, US20060270594; US20060134104; US20060035278; US20050233960; US20050037431
U.S. Pat. No. 5,686,292—for example, ATCC HB-11894 and ATCC HB-11895
US 20100016241—for example, ATCC HB-11894 (hybridoma 1A3.3.13) or HB-11895 (hybridoma 5D5.11.6)

National Defense Medical Center, Taiwan: Lu RM., et al Biomaterials. 2011 Apr; 32(12):3265-74.

Novartis: US20090175860
for example, an antibody comprising the sequences of CDR1, CDR2 and CDR3 of heavy chain 4687, wherein the sequences of CDR1, CDR2, and CDR3 of heavy chain 4687 are residues 26-35, 50-65, and 98-102, respectively, of SEQ ID NO: 58; and the sequences of CDR1, CDR2, and CDR3 of light chain 5097, wherein the sequences of CDR1, CDR2, and CDR3 oflight chain 5097 are residues 24-39,55-61, and 94-100 of SEQ ID NO: 37.

Pharmacia Corporation: US20040166544

Pierre Fabre: US20110239316, US20110097262, US20100115639

Sumsung: US 20110129481—for example a monoclonal antibody produced from a hybridoma cell having accession number KCLRF-BP-00219 or accession number of KCLRF-BP-00223.

Samsung: US 20110104176—for example an antibody produced by a hybridoma cell having Accession Number: KCLRF-BP-00220.

University of Turin Medical School: DN-30 Pacchiana G., et al J Biol Chem. 2010 Nov. 12;285(46):36149-57

Van Andel Research Institute: Jiao Y., et al Mol Biotechnol. 2005 Sep.; 31(1):41-54.

(43) MUC1 (Mucin 1, cell surface associated)

Nucleotide

Genbank accession no J05581

Genbank version no. J05581.1 GI:188869

Genbank record update date: Jun. 23, 2010 08:48 AM

Polypeptide

Genbank accession no. AAA59876

Genbank version no. AAA59876.1 GI:188870

Genbank record update date: Jun. 23, 2010 08:48 AM

Cross References

Gendler S. J., et al J.Biol. Chem. 265 (25), 15286-15293 (1990)

Other Information

Official Symbol: MUC1

Other Aliases: RP11-263K19.2, CD227, EMA, H23AG, KL-6, MAM6, MUC-1, MUC-1/SEC, MUC-1/X, MUC1/ZD, PEM, PEMT, PUM

Other Designations: DF3 antigen; H23 antigen; breast carcinoma-associated antigen DF3; carcinoma-associated mucin; episialin; krebs von den Lungen-6; mucin 1, transmembrane; mucin-1; peanut-reactive urinary mucin; polymorphic epithelial mucin; tumor associated epithelial mucin; tumor-associated epithelial membrane antigen; tumor-associated mucin

ANTIBODIES

AltaRex-Quest Pharma Tech: U.S. 6,716,966—for example an Alt-1 antibody produced by the hybridoma ATCC No PTA-975.

AltaRex-Quest Pharma Tech: U.S. Pat. No. 7,147,850

CRT: 5E5 -Sorensen A L., et al Glycobiology vol. 16 no. 2 pp. 96-107, 2006; HMFG2 —Burchell J., et al Cancer Res., 47, 5476-5482 (1987); see WO2015/159076

Glycotope GT-MAB: GT-MAB 2.5-GEX (Website: http://www.glycotope.com/pipeline/pankomab-gex)

Immunogen: U.S. Pat. No. 7,202,346
for example, antibody MJ-170: hybridoma cell line MJ-170 ATCC accession no. PTA-5286Monoclonal antibody MJ-171: hybridoma cell line MJ-171 ATCC accession no. PTA-5287; monoclonal antibody MJ-172: hybridoma cell line MJ-172 ATCC accession no. PTA-5288; or monoclonal antibody MJ-173: hybridoma cell line MJ-173 ATCC accession no. PTA-5302

Immunomedics: U.S. Pat. No. 6,653,104

Ramot Tel Aviv Uni: U.S. Pat. No. 7,897,351

Regents Uni. CA: U.S. Pat. No. 7,183,388; US20040005647; US20030077676.

Roche GlycArt: U.S. Pat. No. 8,021,856

Russian National Cancer Research Center: Imuteran-Ivanov P K., et al Biotechnol J.2007 Jul.; 2(7):863-70

Technische Univ Braunschweig: (IIB6, HT186-B7, HT186-D11, HT186-G2, HT200-3A-C1, HT220-M-D1, HT220-M-G8)—Thie H., et al PLoS One. 2011 Jan. 14; 6(1):e15921 (44) CA9 (Carbonic anhydrase IX)

Nucleotide

Genbank accession no. X66839

Genbank version no. X66839.1 GI:1000701

Genbank record update date: Feb. 2, 2011 10:15 AM

Polypeptide

Genbank accession no. CAA47315

Genbank version no. CAA47315.1 GI:1000702

Genbank record update date: Feb. 2, 2011 10:15 AM

Cross References

Pastorek J., et al Oncogene 9 (10), 2877-2888 (1994)

Other Information

Official Symbol: CA9

Other Aliases: CAIX, MN

Other Designations: CA-IX; P54/58N; RCC-associated antigen G250; RCC-associated protein G250; carbonate dehydratase IX; carbonic anhydrase 9; carbonic dehydratase; membrane antigen MN; pMW1; renal cell carcinoma-associated antigen G250

ANTIBODIES

Abgenix/Amgen: US20040018198

Affibody: Anti-CAIX Affibody molecules
(http://www.affibody.com/en/Product-Portfolio/Pipeline/)

Bayer: U.S. Pat. No. 7,462,696

Bayer/Morphosys: 3ee9 mAb - Petrul H M., et al Mol Cancer Ther. 2012 Feb.; 11(2):340-9

Harvard Medical School: Antibodies G10, G36, G37, G39, G45, G57, G106, G119, G6, G27, G40 and G125. Xu C., et al PLoS One. 2010 Mar. 10; 5(3):e9625

Institute of Virology, Slovak Academy of Sciences (Bayer)—U.S. Pat. No. 5,955,075
for example, M75- ATCC Accession No. HB 11128 or MN12-ATCC Accession No. HB 11647

Institute of Virology, Slovak Academy of Sciences: U.S. Pat. No. 7,816,493
for example the M75 monoclonal antibody that is secreted from the hybridoma VU-M75, which was deposited at the American Type Culture Collection under ATCC No. HB 11128; or the V/10 monoclonal antibody secreted from the hybridoma V/10-VU, which was deposited at the International Depository Authority of the Belgian Coordinated Collection of Microorganisms (BCCM) at the Laboratorium voor Moleculaire Bioloqie-Plasmidencollectie (LMBP) at the Universeit Gent in Gent, Belgium, under Accession No. LMBP 6009CB.

Institute of Virology, Slovak Academy of Sciences US20080177046; US20080176310; US20080176258; US20050031623

Novartis: US20090252738

Wilex: U.S. Pat. No. 7,691,375-for example the antibody produced by the hybridoma cell line DSM ASC 2526.

Wilex: US20110123537; Rencarex: Kennett R H., et al Curr Opin Mol Ther. 2003 Feb; 5(1):70-5

Xencor: US20090162382

(45) EGFRvIII (Epidermal growth factor receptor (EGFR), transcript variant 3, Nucleotide

Genbank accession no. NM_201283

Genbank version no. NM_201283.1 GI:41327733

Genbank record update date: Sep. 30, 2012 01:47 PM

Polypeptide

Genbank accession no. NP_958440

Genbank version no. NP_958440.1 GI:41327734

Genbank record update date: Sep. 30, 2012 01:47 PM

Cross-References

Batra S K., et al Cell Growth Differ 1995;6:1251-1259.

ANTIBODIES:

U.S. Pat. No. 7,628,986 and U.S. Pat. No. 7,736,644 (Amgen)

For example, a heavy chain variable region amino acid sequence selected from the group consisting of SEQ ID NO: 142 and variants & a light chain variable region amino acid sequence selected from the group consisting of: SEQ ID NO: 144 and variants.

US20100111979 (Amgen)

For example, an antibody comprising a heavy chain amino acid sequence comprising:
CDR1 consisting of a sequence selected from the group consisting of the amino acid sequences for the CDR1 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17); CDR2 consisting of a sequence selected from the group consisting of the amino acid sequences for the CDR2 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17); and

CDR3 consisting of a sequence selected from the group consisting of the amino acid sequences for the CDR3 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

US20090240038 (Amgen)
For example, an antibody having at least one of the heavy or light chain polypeptides comprises an amino acid sequence that is at least 90% identical to the amino acid sequence selected from the group consisting of: SEQ ID NO: 2, SEQ ID NO: 19, SEQ ID NO: 142, SEQ ID NO: 144, and any combination thereof.

US20090175887 (Amgen)
For example, an antibody having a heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

US20090156790 (Amgen)
For example, antibody having heavy chain polypeptide and a light chain polypeptide, wherein at least one of the heavy or light chain polypeptides comprises an amino acid sequence that is at least 90% identical to the amino acid sequence selected from the group consisting of: SEQ ID NO: 2, SEQ ID NO: 19, SEQ ID NO: 142, SEQ ID NO: 144, and any combination thereof.

US20090155282, US20050059087 and US20050053608 (Amgen)
For example, an antibody heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095

(SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

MR1-1 (U.S. Pat. No. 7,129,332; Duke)
For example, a variant antibody having the sequence of SEQ ID NO.18 with the substitutions S98P-T99Y in the CDR3 VH, and F92W in CDR3 VL.

L8A4, H10, Y10 (Wikstrand C J., et al Cancer Res. 1995 Jul. 15;55(14):3140-8; Duke)

US20090311803 (Harvard University)
For example, SEQ ID NO:9 for antibody heavy chain variable region, and SEQ ID NO: 3 for light chain variable region amino acid sequences

US20070274991 (EMD72000, also known as matuzumab; Harvard University)
For example, SEQ ID NOs: 3 & 9 for light chain and heavy chain respectively

U.S. Pat. No. 6,129,915 (Schering)

For example, SEQ. ID NOs: 1, 2, 3, 4, 5 and 6.

mAb CH12-Wang H., et al FASEB J.2012 Jan.;26(1):73-80 (Shanghai Cancer Institute).

RAbDMvIII-Gupta P., et al BMC Biotechnol. 2010 Oct. 7;10:72 (Stanford University Medical Center).

mAb Ua30-Ohman L., et al Tumour Biol. 2002 Mar.-Apr.; 23(2):61-9 (Uppsala University).

Han D G., et al Nan Fang Yi Ke Da Xue Xue Bao. 2010 Jan.;30(1):25-9 (Xi'an Jiaotong University).

(46) CD33 (CD33 molecule)

Nucleotide

Genbank accession no. M_23197

Genbank version no. NM__23197.1 GI:180097

Genbank record update date: Jun. 23, 2010 08:47 AM

Polypeptide

Genbank accession no. AAA51948

Genbank version no. AAA51948.1 GI:188098

Genbank record update date: Jun. 23, 2010 08:47 AM

Cross-References

Simmons D., et al J. Immunol. 141 (8), 2797-2800 (1988)

Other Information

Official Symbol: CD33

Other Aliases: SIGLEC-3, SIGLEC3, p67

Other Designations: CD33 antigen (gp67); gp67; myeloid cell surface antigen CD33; sialic acid binding Ig-like lectin 3; sialic acid-binding Ig-like lectin

ANTIBODIES

H195 (Lintuzumab)- Raza A., et al Leuk Lymphoma. 2009 Aug.;50(8):1336-44;

U.S. Pat. No. 6,759,045 (Seattle Genetics/Immunomedics)

mAb OKT9: Sutherland, D. R. et al. Proc Natl Acad Sci USA 78 (7): 4515-4519 1981, Schneider,C., et al J Biol Chem 257, 8516-8522 (1982) mAb E6: Hoogenboom,H. R., et al J Immunol 144, 3211-3217 (1990)

U.S. 6,590,088 (Human Genome Sciences)

For example, SEQ ID NOs: 1 and 2 and ATCC accession no. 97521

U.S. Pat. No. 7,557,189 (Immunogen)

For example, an antibody or fragment thereof comprising a heavy chain variable region which comprises three CDRs having the amino acid sequences of SEQ ID NOs:1-3 and a light chain variable region comprising three CDRs having the amino acid sequences of SEQ ID NOs:4-6.

(47) CD19 (CD19 molecule)

Nucleotide

Genbank accession no. NM_001178098

Genbank version no. NM_001178098.1 GI:296010920

Genbank record update date: Sep. 10, 2012 12:43 AM

Polypeptide

Genbank accession no. NP_001171569

Genbank version no. NP_001171569.1 GI:296010921

Genbank record update date: Sep. 10, 2012 12:43 AM

Cross-References Tedder T F., et al J. Immunol. 143 (2): 712-7 (1989)

Other Information

Official Symbol: CD19

Other Aliases: B4, CVID3

Other Designations: B-lymphocyte antigen CD19; B-lymphocyte surface antigen B4; T-cell surface antigen Leu-12; differentiation antigen CD19

ANTIBODIES

Immunogen: HuB4 - Al-Katib A M., et al Clin Cancer Res. 2009 Jun. 15;15(12):4038-45.

4G7: Kugler M., et al Protein Eng Des Sel. 2009 Mar.;22(3):135-47

For example, sequences in FIG. 3 of of Knappik, A. et al. J Mol Biol 2000 Feb.; 296(1):57-86

AstraZeneca/Medlmmune: MEDI-551-Herbst R., et al J Pharmacol Exp Ther. 2010 Oct.;335(1):213-22

Glenmark Pharmaceuticals: GBR-401-Hou S., et al Mol Cancer Ther November 2011 (Meeting Abstract Supplement) C164

U.S. Pat. No. 7,109,304 (Immunomedics)
For example, an antibody comprising the sequence of hA19Vk (SEQ ID NO:7) and the sequence of hA19VH (SEQ ID NO:10)

U.S. Pat. No. 7,902,338 (Immunomedics)
For example, an antibody or antigen-binding fragment thereof that comprises the light chain complementarity determining region CDR sequences CDR1 of SEQ ID NO: 16 (KASQSVDYDGDSYLN); CDR2 of SEQ ID NO: 17 (DASNLVS); and CDR3 of SEQ ID NO: 18 (QQSTEDPWT) and the heavy chain CDR sequences CDR1 of SEQ ID NO: 19 (SYWMN); CDR2 of SEQ ID NO: 20 (QIWPGDGDTNYNGKFKG) and CDR3 of SEQ ID NO: 21 (RETTTVGRYYYAMDY) and also comprises human antibody framework (FR) and constant region sequences with one or more framework region amino acid residues substituted from the corresponding framework region sequences of the parent murine antibody, and wherein said substituted FR residues comprise the substitution of serine for phenylalanine at Kabat residue 91 of the heavy chain variable region.

Medarex: MDX-1342 —Cardarelli P M., et al Cancer Immunol Immunother. 2010 Feb.; 59(2):257-65.

MorphoSys/Xencor: MOR-208/XmAb-5574-Zalevsky J., et al Blood. 2009 Apr. 16; 113(16):3735-43

U.S. Pat. No. 7,968,687 (Seattle Genetics)

An antibody or antigen-binding fragment comprising a heavy chain variable domain comprising the amino acid sequence of SEQ ID NO:9 and a light chain variable domain comprising the amino acid sequence of SEQ ID NO: 24.

4G7chim-Lang P., et al Blood. 2004 May 15; 103(10):3982-5 (University of Tubingen)
For example, FIG. 6 and SEQ ID No: 80 of US20120082664

Zhejiang University School of Medicine: 2E8- Zhang J., et al J Drug Target. 2010

Nov.; 18(9):675-8

(48) IL2RA (Interleukin 2 receptor, alpha); NCBI Reference Sequence: NM_000417.2);

Nucleotide

Genbank accession no. NM_000417

Genbank version no. NM_000417.2 GI:269973860

Genbank record update date: Sep. 09, 2012 04:59 PM

Polypeptide

Genbank accession no. NP_000408

Genbank version no. NP_000408.1 GI:4557667

Genbank record update date: Sep. 09, 2012 04:59 PM

Cross-References

Kuziel W. A., et al J. Invest. Dermatol. 94 (6 SUPPL), 27S-32S (1990)

Other Information

Official Symbol: IL2RA

Other Aliases: RP11-536K7.1, CD25, IDDM10, IL2R, TCGFR

Other Designations: FIL-2 receptor subunit alpha; IL-2-RA; IL-2R subunit alpha; IL2-RA;

TAC antigen; interleukin-2 receptor subunit alpha; p55

ANTIBODIES

U.S. Pat. No.6,383,487 (Novartis/UCL: Baxilisimab [Simulect])

U.S. Pat. No.6,521,230 (Novartis/UCL: Baxilisimab [Simulect])
For example, an antibody having an antigen binding site comprises at least one domain which comprises CDR1 having the amino acid sequence in SEQ. ID. NO: 7, CDR2 having the amino acid sequence in SEQ. ID. NO: 8, and CDR3 chaving the amino acid sequence in SEQ. ID. NO: 9; or said CDR1, CDR2 and CDR3 taken in sequence as a whole comprise an amino acid sequence which is at least 90% identical to SEQ. ID. NOs: 7, 8 and 9 taken in sequence as a whole.

Daclizumab-Rech A J., et al Ann N Y Acad Sci. 2009 Sep.; 1174:99-106 (Roche)

(49) AXL (AXL receptor tyrosine kinase)

Nucleotide

Genbank accession no. M76125

Genbank version no. M76125.1 GI:292869

Genbank record update date: Jun. 23, 2010 08:53 AM

Polypeptide

Genbank accession no. AAA61243

Genbank version no. AAA61243.1 GI:29870

Genbank record update date: Jun. 23, 2010 08:53 AM

Cross-References

O'Bryan J. P., et al Mol. Cell. Biol. 11 (10), 5016-5031 (1991); Bergsagel P. L., et al J. Immunol. 148 (2), 590-596 (1992)

Other Information

Official Symbol: AXL

Other Aliases: JTK11, UFO

Other Designations: AXL oncogene; AXL transforming sequence/gene; oncogene AXL; tyrosine-protein kinase receptor UFO

ANTIBODIES

YW327.6S2 - Ye X., et al Oncogene. 2010 Sep. 23; 29(38):5254-64. (Genentech)

BergenBio: BGB324 (http://www.bergenbio.com/BGB324)

(50) CD30-TNFRSF8 (Tumor necrosis factor receptor superfamily, member 8)

Nucleotide

Genbank accession no. M83554

Genbank version no. M83554.1 GI:180095

Genbank record update date: Jun. 23, 2010 08:53 AM

Polypeptide

Genbank accession no. AAA51947

Genbank version no. AAA51947.1 GI:180096

Genbank record update date: Jun. 23, 2010 08:53 AM

Cross-References Durkop H., et al Cell 68 (3), 421-427 (1992)

Other information

Official Symbol: TNFRSF8

Other Aliases: CD30, D1S166E, Ki-1

Other Designations: CD3OL receptor; Ki-1 antigen; cytokine receptor CD30; lymphocyte activation antigen CD30; tumor necrosis factor receptor superfamily member 8 (51) BCMA (B-cell maturation antigen)-TNFRSF17 (Tumor necrosis factor receptor superfamily, member 17)

Nucleotide

Genbank accession no. Z29574

Genbank version no. Z29574.1 GI:471244

Genbank record update date: Feb. 02, 2011 10:40 AM

Polypeptide

Genbank accession no. CAA82690

Genbank version no. CAA82690.1 GI:471245

Genbank record update date: Feb. 02, 2011 10:40 AM

Cross-References

Laabi Y., et al Nucleic Acids Res. 22 (7), 1147-1154 (1994)

Other Information

Official Symbol: TNFRSF17

Other Aliases: BCM, BCMA, CD269

Other Designations: B cell maturation antigen; B-cell maturation factor; B-cell maturation protein; tumor necrosis factor receptor superfamily member 17

(52) CT Ags-CTA (Cancer Testis Antigens)

Cross-References

Fratta E., et al. Mol Oncol. 2011 Apr.; 5(2):164-82; Lim S H., at al Am J Blood Res. 2012; 2(1):29-35.

(53) CD174 (Lewis Y)-FUT3 (fucosyltransferase 3 (galactoside 3(4)-L-fucosyltransferase, Lewis blood group)

Nucleotide

Genbank accession no. NM000149

Genbank version no. NM000149.3 GI:148277008

Genbank record update date: Jun. 26, 2012 04:49 PM

Polypeptide Genbank accession no. NP_000140

Genbank version no. NP_000140.1 GI:4503809

Genbank record update date: Jun. 26, 2012 04:49 PM

Cross-References

Kukowska-Latallo, J. F., et al Genes Dev. 4 (8), 1288-1303 (1990)

Other Information

Official Symbol: FUT3

Other Aliases: CD174, FT3B, FucT-Ill, LE, Les

Other Designations: Lewis F T; alpha-(1,3/1,4)-fucosyltransferase; blood group Lewis alpha-4-fucosyltransferase; fucosyltransferase III; galactoside 3(4)-L-fucosyltransferase (54) CLEC14A (C-type lectin domain family 14, member A; Genbank accession no. NM175060)

Nucleotide

Genbank accession no. NM175060

Genbank version no. NM175060.2 GI:371123930

Genbank record update date: Apr 01, 2012 03:34 PM

Polypeptide

Genbank accession no. NP_778230

Genbank version no. NP_778230.1 GI:28269707

Genbank record update date: Apr. 01, 2012 03:34 PM

Other Information

Official Symbol: CLEC14A

Other Aliases: UNQ236/PRO269, C14orf27, CEG1, EGFR-5

Other Designations: C-type lectin domain family 14 member A; CIECT and EGF-like domain containing protein; epidermal growth factor receptor 5

(55) GRP78-HSPAS (heat shock 70 kDa protein 5 (glucose-regulated protein, 78 kDa)

Nucleotide

Genbank accession no. NM005347

Genbank version no. NM005347.4 GI:305855105

Genbank record update date: Sep. 30, 2012 01:42 PM

Polypeptide

Genbank accession no. NP_005338

Genbank version no. NP_005338.1 GI:16507237

Genbank record update date: Sep. 30, 2012 01:42 PM

Cross-References

Ting J., et al DNA 7 (4), 275-286 (1988)

Other Infromation

Official Symbol: HSPAS

Other Aliases: BIP, GRP78, MIF2

Other Designations: 78 kDa glucose-regulated protein; endoplasmic reticulum lumenal Ca(2+)-binding protein grp78; immunoglobulin heavy chain-binding protein (56) CD70 (CD70 molecule) L08096

Nucleotide

Genbank accession no. L08096

Genbank version no. L08096.1 GI:307127

Genbank record update date: Jun. 23, 2012 08:54 AM

Polypeptide

Genbank accession no. AAA36175

Genbank version no. AAA36175.1 GI:307128

Genbank record update date: Jun. 23, 2012 08:54 AM

Cross-References

Goodwin R. G., et al Cell 73 (3), 447-456 (1993)

Other Information

Official Symbol: CD70

Other Aliases: CD27L, CD27LG, TNFSF7

Other Designations: CD27 ligand; CD27-L; CD70 antigen; Ki-24 antigen; surface antigen CD70; tumor necrosis factor (ligand) superfamily, member 7; tumor necrosis factor ligand superfamily member 7

ANTIBODIES

MDX-1411 against CD70 (Medarex)

h1F6 (Oflazoglu, E., et al, Clin Cancer Res. 2008 Oct. 1; 14(19):6171-80; Seattle Genetics)
For example, see US20060083736 SEQ ID NOs: 1, 2, 11 and 12 and FIG. 1.

(57) Stem Cell specific antigens. For example:
5T4 (see entry (63) below)
CD25 (see entry (48) above)
CD32
   Polypeptide
      Genbank accession no. ABK42161
      Genbank version no. ABK42161.1 GI:117616286
      Genbank record update date: Jul. 25, 2007 03:00 PM
LGR5/GPR49
   Nucleotide
      Genbank accession no. NM_003667
      Genbank version no. NM_003667.2 GI:24475886
      Genbank record update date: Jul. 22, 2012 03:38 PM
   Polypeptide
      Genbank accession no. NP_003658
      Genbank version no. NP_003658.1 GI:4504379
      Genbank record update date: Jul. 22, 2012 03:38 PM
Prominin/CD133
   Nucleotide
      Genbank accession no. NM_006017
      Genbank version no. NM_006017.2 GI:224994187
      Genbank record update date: Sep. 30, 2012 01:47 PM
   Polypeptide
      Genbank accession no. NP_006008
      Genbank version no. NP_006008.1 GI:5174387
      Genbank record update date: Sep. 30, 2012 01:47 PM

(58) ASG-5

Cross-References

(Smith L. M., et.al AACR 2010 Annual Meeting (abstract #2590); Gudas J. M., et.al. AACR 2010 Annual Meeting (abstract #4393)

ANTIBODIES

Anti-AGS-5 Antibody: M6.131 (Smith, L. M., et.al AACR 2010 Annual Meeting (abstract #2590)

(59) ENPP3 (Ectonucleotide pyrophosphatase/phosphodiesterase 3)

Nucleotide

Genbank accession no. AF005632

Genbank version no. AF005632.2 GI:4432589

Genbank record update date: Mar. 10, 2010 09:41 PM

Polypeptide

Genbank accession no. AAC51813

Genbank version no. AAC51813.1 GI:2465540

Genbank record update date: Mar. 10, 2010 09:41 PM

Cross-References

Jin-Hua P., et al Genomics 45 (2), 412-415 (1997)

Other information

Official Symbol: ENPP3

Other Aliases: RP5-988G15.3, B10, CD203c, NPP3, PD-IBETA, PDNP3

Other Designations: E-NPP 3; dJ1005H11.3 (phosphodiesterase I/nucleotide pyrophosphatase 3); dJ914N13.3 (phosphodiesterase I/nucleotide pyrophosphatase 3); ectonucleotide pyrophosphatase/phosphodiesterase family member 3; gp13ORB13-6; phosphodiesterase I beta; phosphodiesterase I/nucleotide pyrophosphatase 3;

phosphodiesterase-I beta

(60) PRR4 (Proline rich 4 (lacrimal))

Nucleotide

Genbank accession no. NM_007244

Genbank version no. NM_007244.2 GI:154448885

Genbank record update date: Jun. 28, 2012 12:39 PM

Polypeptide

Genbank accession no. NP_009175

Genbank version no. NP_009175.2 GI:154448886

Genbank record update date: Jun. 28, 2012 12:39 PM

Cross-References

Dickinson D. P., et al Invest. Ophthalmol. Vis. Sci. 36 (10), 2020-2031 (1995)

Other Information

Official Symbol: PRR4

Other Aliases: LPRP, PROL4

Other Designations: lacrimal proline-rich protein; nasopharyngeal carcinoma-associated proline-rich protein 4; proline-rich polypeptide 4; proline-rich protein 4

(61) GCC-GUCY2C (guanylate cyclase 2C (heat stable enterotoxin receptor)

Nucleotide

Genbank accession no. NM_004963

Genbank version no. NM_004963.3 GI:222080082

Genbank record update date: Sep. 02, 2012 01:50 PM

Polypeptide

Genbank accession no. NP_004954

Genbank version no. NP_004954.2 GI:222080083

Genbank record update date: Sep. 02, 2012 01:50 PM

Cross-References

De Sauvage F. J., et al J.Biol. Chem. 266 (27), 17912-17918 (1991); Singh S., et al Biochem. Biophys. Res. Commun. 179 (3), 1455-1463 (1991)

Other Information

Official Symbol: GUCY2C

Other Aliases: DIAR6, GUC2C, MUCIL, STAR

Other Designations: GC-C; STA receptor; guanylyl cyclase C; hSTAR; heat-stable enterotoxin receptor; intestinal guanylate cyclase

(62) Liv-1-SLC39A6 (Solute carrier family 39 (zinc transporter), member 6)

Nucleotide

Genbank accession no. U41060

Genbank version no. U41060.2 GI:12711792

Genbank record update date: Nov. 30, 2009 04:35 PM

Polypeptide

Genbank accession no. AAA96258

Genbank version no. AAA96258.2 GI:12711793

Genbank record update date: Nov. 30, 2009 04:35 PM

Cross-References

Taylor K M., et al Biochim Biophys Acta. 2003 Apr. 1; 1611(1-2):16-30

Other Information

Official Symbol: SLC39A6

Other Aliases: LIV-1

Other Designations: LIV-1 protein, estrogen regulated; ZIP-6; estrogen-regulated protein LIV-1; solute carrier family 39 (metal ion transporter), member 6; solute carrier family 39 member 6; zinc transporter ZIP6; zrt- and Irt-like protein 6

(63) 5T4, Trophoblast glycoprotein, TPBG-TPBG (trophoblast glycoprotein)

Nucleotide

Genbank accession no. AJ012159

Genbank version no. AJ012159.1 GI:3805946

Genbank record update date: Feb. 01, 2011 10:27 AM

Polypeptide

Genbank accession no. CAA09930

Genbank version no. CAA09930.1 GI:3805947

Genbank record update date: Feb. 01, 2011 10:27 AM

Cross-References

King K. W.,et al Biochim. Biophys. Acta 1445 (3), 257-270 (1999)

Other Information

Official Symbol: TPBG

Other Aliases: 5T4, 5T4AG, M6P1

Other Designations: 5T4 oncofetal antigen; 5T4 oncofetal trophoblast glycoprotein; 5T4 oncotrophoblast glycoprotein

See WO2015/155345

(64) CD56-NCMA1 (Neural cell adhesion molecule 1)

Nucleotide

Genbank accession no. NM_000615

Genbank version no. NM_000615.6 GI:336285433

Genbank record update date: Sep. 23, 2012 02:32 PM

Polypeptide

Genbank accession no. NP_000606

Genbank version no. NP_000606.3 GI:94420689

Genbank record update date: Sep. 23, 2012 02:32 PM

Cross-References

Dickson,G., et al, Cell 50 (7), 1119-1130 (1987)

Other Information

Official Symbol: NCAM1

Other Aliases: CD56, MSK39, NCAM

Other Designations: antigen recognized by monoclonal antibody 5.1H11; neural cell adhesion molecule, NCAM

ANTIBODIES

Immunogen: HuN901 (Smith S V., et al Curr Opin Mol Ther. 2005 Aug.; 7(4):394-401)
For example, see humanized from murine N901 antibody. See FIGS. 1b and 1e of Roguska, M. A., et al. Proc Natl Acad Sci USA Feb. 1994; 91:969-973.

(65) CanAg (Tumor associated antigen CA242)

Cross-References

Haglund C., et al Br J Cancer 60:845-851, 1989;Baeckstrom D., et al J Biol Chem 266:21537-21547, 1991

ANTIBODIES

huC242 (Tolcher A W et al., J Clin Oncol. 2003 Jan. 15; 21(2):211-22; Immunogen)
For example, see US20080138898A1 SEQ ID NO: 1 and 2

(66) FOLR1 (Folate Receptor 1)

Nucleotide

Genbank accession no. J05013

Genbank version no. J05013.1 GI:182417

Genbank record update date: Jun. 23, 2010 08:47 AM

Polypeptide

Genbank accession no. AAA35823

Genbank version no. AAA35823.1 GI:182418

Genbank record update date: Jun. 23, 2010 08:47 AM

Cross-References

Elwood P. C., et al J. Biol. Chem. 264 (25), 14893-14901 (1989)

Other Information

Official Symbol: FOLR1

Other Aliases: FBP, FOLR

Other Designations: FR-alpha; KB cells FBP; adult folate-binding protein; folate binding protein; folate receptor alpha; folate receptor, adult; ovarian tumor-associated antigen

MOv18

ANTIBODIES

M9346A-Whiteman K R., et al Cancer Res Apr. 15, 2012; 72(8 Supplement): 4628(Immunogen)

(67) GPNMB (Glycoprotein (transmembrane) nmb)

Nucleotide

Genbank accession no. X76534

Genbank version no. X76534.1 GI:666042

Genbank record update date: Feb. 02, 2011 10:10 AM

Polypeptide

Genbank accession no. CAA54044

Genbank version no. CAA54044.1 GI:666043

Genbank record update date: Feb. 02, 2011 10:10 AM

Cross-References

Weterman M. A., et al Int. J. Cancer 60 (1), 73-81 (1995)

Other Information

Official Symbol: GPNMB

Other Aliases: UNQ1725/PRO9925, HGFIN, NMB

Other Designations: glycoprotein NMB; glycoprotein nmb-like protein; osteoactivin; transmembrane glycoprotein HGFIN; transmembrane glycoprotein NMB

ANTIBODIES

Celldex Therapeutics: CR011 (Tse K F., et al Clin Cancer Res. 2006 Feb. 15; 12(4):1373-82)
For example, see EP1827492B1 SEQ ID NO: 22, 24, 26, 31, 33 and 35

(68) TIM-1-HAVCR1 (Hepatitis A virus cellular receptor 1)

Nucleotide

Genbank accession no. AF043724

Genbank version no. AF043724.1 GI:2827453

Genbank record update date: Mar. 10, 2010 06:24 PM

Polypeptide

Genbank accession no. AAC39862

Genbank version no. AAC39862.1 GI:2827454

Genbank record update date: Mar. 10, 2010 06:24 PM

Cross-References

Feigelstock D., et al J. Virol. 72 (8), 6621-6628 (1998)

Other Information

Official Symbol: HAVCR1

Other Aliases: HAVCR, HAVCR-1, KIM-1, KIM1, TIM, TIM-1, TIM1, TIMD-1, TIMD1

Other Designations: T cell immunoglobin domain and mucin domain protein 1; T-cell membrane protein 1; kidney injury molecule 1

(69) RG-1/Prostate tumor target Mindin-Mindin/RG-1

Cross-References

Parry R., et al Cancer Res. 2005 Sep. 15; 65(18):8397-405

(70) B7-H4-VTCN1 (V-set domain containing T cell activation inhibitor 1

Nucleotide

Genbank accession no. BX648021

Genbank version no. BX648021.1 GI:34367180

Genbank record update date: Feb. 02, 2011 08:40 AM

Cross-References

Sica GL., et al Immunity. 2003 Jun.; 18(6):849-61

Other Information

Official Symbol: VTCN1

Other Aliases: RP11-229A19.4, B7-H4, B7H4, B7S1, B7X, B7h.5, PRO1291, VCTN1

Other Designations: B7 family member, H4; B7 superfamily member 1; T cell costimulatory molecule B7x; T-cell costimulatory molecule B7x; V-set domain-containing T-cell activation inhibitor 1; immune costimulatory protein B7-H4

(71) PTK7 (PTK7 protein tyrosine kinase 7)

Nucleotide

Genbank accession no. AF447176

Genbank version no. AF447176.1 GI:17432420

Genbank record update date: Nov. 28, 2008 01:51 PM

Polypeptide

Genbank accession no. AAL39062

Genbank version no. AAL39062.1 GI:17432421

Genbank record update date: Nov. 28, 2008 01:51 PM

Cross-References

Park S. K.,et al J.Biochem. 119 (2), 235-239 (1996)

Other Information

Official Symbol: PTK7

Other Aliases: CCK-4, CCK4

Other Designations: colon carcinoma kinase 4; inactive tyrosine-protein kinase 7; pseudo tyrosine kinase receptor 7; tyrosine-protein kinase-like 7

(72) CD37 (CD37 molecule)

Nucleotide

Genbank accession no. NM_001040031

Genbank version no. NM_001040031.1 GI:91807109

Genbank record update date: Jul. 29, 2012 02:08 PM

Polypeptide

Genbank accession no. NP_001035120

Genbank version no. NP_001035120.1 GI:91807110

Genbank record update date: Jul. 29, 2012 02:08 PM

Cross-References

Schwartz-Albiez R., et al J. Immunol. 140 (3), 905-914 (1988)

Other Information

Official Symbol: CD37

Other Aliases: GP52-40, TSPAN26

Other Designations: CD37 antigen; cell differentiation antigen 37; leukocyte antigen CD37; leukocyte surface antigen CD37; tetraspanin-26; tspan-26

ANTIBODIES

Boehringer Ingelheim: mAb 37.1 (Heider K H., et al Blood. 2011 Oct. 13; 118(15):4159-68)

Trubion: CD37-SMIP (G28-1 scFv-Ig) ((Zhao X., et al Blood. 2007; 110: 2569-2577)

For example, see US20110171208A1 SEQ ID NO: 253

Immunogen: K7153A (Deckert J., et al Cancer Res Apr. 15, 2012; 72(8 Supplement): 4625)

(73) CD138 -SDC1 (syndecan 1)

Nucleotide

Genbank accession no. AJ551176

Genbank version no. AJ551176.1 GI:29243141

Genbank record update date: Feb. 01, 2011 12:09 PM

Polypeptide

Genbank accession no. CAD80245

Genbank version no. CAD80245.1 GI:29243142

Genbank record update date: Feb. 01, 2011 12:09 PM

Cross-References

O'Connell F P., et al Am J Clin Pathol. 2004 Feb.;121(2):254-63

Other Information

Official Symbol: SDC1

Other Aliases: CD138, SDC, SYND1, syndecan

Other Designations: CD138 antigen; heparan sulfate proteoglycan fibroblast growth factor receptor; syndecan proteoglycan 1; syndecan-1

ANTIBODIES

Biotest: chimerized MAb (nBT062)-(Jagannath S., et al Poster ASH #3060, 2010; WIPO Patent Application WO/2010/128087)
For example, see US20090232810 SEQ ID NO: 1 and 2

Immunogen: B-B4 (Tassone P., et al Blood 104_3688-3696)
For example, see US20090175863A1 SEQ ID NO: 1 and 2

(74) CD74 (CD74 molecule, major histocompatibility complex, class II invariant chain)

Nucleotide

Genbank accession no. NM_004355

Genbank version no. NM_004355.1 GI:343403784

Genbank record update date: Sep. 23, 2012 02:30 PM

Polypeptide

Genbank accession no. NP_004346

Genbank version no. NP_004346.1 GI:10835071

Genbank record update date: Sep. 23, 2012 02:30 PM

Cross-References

Kudo, J., et al Nucleic Acids Res. 13 (24), 8827-8841 (1985)

Other Information

Official Symbol: CD74

Other Aliases: DHLAG, HLADG, II, Ia-GAMMA

Other Designations: CD74 antigen (invariant polypeptide of major histocompatibility complex, class II antigen-associated); HLA class II histocompatibility antigen gamma chain; HLA-DR antigens-associated invariant chain; HLA-DR-gamma; Ia-associated invariant chain; MHC HLA-DR gamma chain; gamma chain of class II antigens; p33

ANTIBODIES

Immunomedics: hLL1 (Milatuzumab,)-Berkova Z., et al Expert Opin Investig Drugs. 2010 Jan.; 19(1):141-9)
For example, see US20040115193 SEQ ID NOs: 19, 20, 21, 22, 23 and 24

Genmab: HuMax-CD74 (see website)

(75) Claudins—CLs (Claudins)

Cross-References

Offner S., et al Cancer Immunol Immunother. 2005 May; 54(5):431-45, Suzuki H., et al Ann N Y Acad Sci. 2012 Jul.;1258:65-70)

In humans, 24 members of the family have been described—see literature reference.

(76) EGFR (Epidermal growth factor receptor)

Nucleotide

Genbank accession no. NM_005228

Genbank version no. NM_005228.3 GI:41927737

Genbank record update date: Sep. 30, 2012 01:47 PM

Polypeptide

Genbank accession no. NP_005219

Genbank version no. NP_005219.2 GI:29725609

Genbank record update date: Sep. 30, 2012 01:47 PM

Cross-References

Dhomen N S., et al Crit Rev Oncog. 2012; 17(1):31-50

Other Information

Official Symbol: EGFR

Other Aliases: ERBB, ERBB1, HER1, PIG61, mENA

Other Designations: avian erythroblastic leukemia viral (v-erb-b) oncogene homolog; cell growth inhibiting protein 40; cell proliferation-inducing protein 61; proto-oncogene c-ErbB-1; receptor tyrosine-protein kinase erbB-1

ANTIBODIES

BMS: Cetuximab (Erbitux)-Broadbridge V T., et al Expert Rev Anticancer Ther. 2012

May; 12(5):555-65.
For example, see U.S. Pat. No. 6,217,866-ATTC deposit No. 9764.

Amgen: Panitumumab (Vectibix)-Argiles G., et al Future Oncol. 2012 Apr.; 8(4):373-89
For example, see U.S. Pat. No. 6,235,883 SEQ ID NOs: 23-38.

Genmab: Zalutumumab-Rivera F., et al Expert Opin Biol Ther. 2009 May; 9(5):667-74.

Y M Biosciences: Nimotuzumab-Ramakrishnan M S., et al MAbs. 2009 Jan.-Feb.; 1(1):41-8.
For example, see U.S. Pat. No. 5,891,996 SEQ ID NOs: 27-34.

(77) Her3(ErbB3)-ERBB3 (v-erb-b2 erythroblastic leukemia viral oncogene homolog 3(avian))

Nucleotide

Genbank accession no. M34309

Genbank version no. M34309.1 GI:183990

Genbank record update date: Jun. 23, 2010 08:47 PM

Polypeptide

Genbank accession no. AAA35979

Genbank version no. AAA35979.1 GI:306841

Genbank record update date: Jun. 23, 2010 08:47 PM

Cross-References

Plowman,G. D., et al., Proc. Natl. Acad. Sci. U.S.A. 87 (13), 4905-4909 (1990)

Other Information

Official Symbol: ERBB3

Other Aliases: ErbB-3, HER3, LCCS2, MDA-BF-1, c-erbB-3, c-erbB3, erbB3-S, p180- ErbB3, p45-sErbB3, p85-sErbB3

Other Designations: proto-oncogene-like protein c-ErbB-3; receptor tyrosine-protein kinase erbB-3; tyrosine kinase-type cell surface receptor HER3

ANTIBODIES

Merimack Pharma: MM-121 (Schoeberl B., et al Cancer Res. 2010 Mar. 15;70(6):2485-2494)

For example, see US2011028129 SEQ ID NOs: 1, 2, 3, 4, 5, 6, 7 and 8.

(78) RON-MST1R (macrophage stimulating 1 receptor (c-met-related tyrosine kinase))

Nucleotide

Genbank accession no. X70040

Genbank version no. X70040.1 GI:36109

Genbank record update date: Feb. 02, 2011 10:17 PM

Polypeptide

Genbank accession no. CCA49634

Genbank version no. CCA49634.1 GI:36110

Genbank record update date: Feb. 02, 2011 10:17 PM

Cross-References

Ronsin C., et al Oncogene 8 (5), 1195-1202 (1993)

Other Information

Official Symbol: MST1R

Other Aliases: CD136, CDw136, PTK8, RON

Other Designations: MSP receptor; MST1 R variant RON30; MST1R variant RON62; PTK8 protein tyrosine kinase 8; RON variant E2E3; c-met-related tyrosine kinase; macrophage-stimulating protein receptor; p185-Ron; soluble RON variant 1; soluble RON variant 2; soluble RON variant 3; soluble RONvariant 4

(79) EPHA2 (EPH receptor A2)

Nucleotide

Genbank accession no. BC037166

Genbank version no. BC037166.2 GI:33879863

Genbank record update date: Mar. 06, 2012 01:59 PM

Polypeptide

Genbank accession no. AAH37166

Genbank version no. AAH37166.1 GI:22713539

Genbank record update date: Mar. 06, 2012 01:59 PM

Cross-References

Strausberg R. L., et al Proc. Natl. Acad. Sci. U.S.A. 99 (26), 16899-16903 (2002)

Other Information

Official Symbol: EPHA2

Other Aliases: ARCC2, CTPA, CTPP1, ECK

Other Designations: ephrin type-A receptor 2; epithelial cell receptor protein tyrosine kinase; soluble EPHA2 variant 1; tyrosine-protein kinase receptor ECK

ANTIBODIES

Medimmune: 1C1 (Lee J W., et al Clin Cancer Res. 2010 May 1; 16(9):2562-2570)

For example, see US20090304721A1 FIGS. 7 and 8.

(80) CD20-MS4A1 (membrane-spanning 4-domains, subfamily A, member 1)

Nucleotide

Genbank accession no. M27394

Genbank version no. M27394.1 GI:179307

Genbank record update date: Nov. 30, 2009 11:16 AM

Polypeptide

Genbank accession no. AAA35581

Genbank version no. AAA35581.1 GI:179308

Genbank record update date: Nov. 30, 2009 11:16 AM

Cross-References

Tedder T. F., et al Proc. Natl. Acad. Sci. U.S.A. 85 (1), 208-212 (1988)

Other Information

Official Symbol: MS4A1

Other Aliases: B1, Bp35, CD20, CVIDS, LEU-16, MS4A2, S7

Other Designations: B-lymphocyte antigen CD20; B-lymphocyte cell-surface antigen B1;

CD20 antigen; CD20 receptor; leukocyte surface antigen Leu-16

ANTIBODIES

Genentech/Roche: Rituximab-Abdulla N E., et al BioDrugs. 2012 Apr. 1;26(2):71-82.

For example, see U.S. Pat. No. 5,736,137, ATCC deposit No. HB-69119.

GSK/Genmab: Ofatumumab-Nightingale G., et al Ann Pharmacother. 2011

Oct.; 45(10):1248-55.

For example, see US20090169550A1 SEQ ID NOs: 2, 4 and 5.

Immunomedics: Veltuzumab-Goldenberg D M., et al Leuk Lymphoma. 2010 May; 51(5):747-55.

For example, see U.S. Pat. No. 7,919,273B2 SEQ ID NOs: 1, 2, 3, 4, 5 and 6.

(81) Tenascin C-TNC (Tenascin C)

Nucleotide

Genbank accession no. NM_002160

Genbank version no. NM_002160.3 GI:340745336

Genbank record update date: Sep. 23, 2012 02:33 PM

Polypeptide

Genbank accession no. NP_002151

Genbank version no. NP_002151.2 GI:153946395

Genbank record update date: Sep. 23, 2012 02:33 PM

Cross-References

Nies D. E., et al J. Biol. Chem. 266 (5), 2818-2823 (1991); Siri A., et al Nucleic Acids Res. 19 (3), 525-531 (1991)

Other Information

Official Symbol: TNC

Other Aliases: 150-225, GMEM, GP, HXB, JI, TN, TN—C

Other Designations: GP 150-225; cytotactin; glioma-associated-extracellular matrix antigen; hexabrachion (tenascin); myotendinous antigen; neuronectin; tenascin; tenascin-C isoform 14/AD1/16

ANTIBODIES

Philogen: G11 (von Lukowicz T., et al J Nucl Med. 2007 Apr.; 48(4):582-7) and F16 (Pedretti M., et al Lung Cancer. 2009 Apr.; 64(1):28-33)

For example, see U.S. 7,968,685 SEQ ID NOs: 29, 35, 45 and 47.

(82) FAP (Fibroblast activation protein, alpha)

Nucleotide

Genbank accession no. U09278

Genbank version no. U09278.1 GI:1888315

Genbank record update date: Jun. 23, 2010 09:22 AM

Polypeptide

Genbank accession no. AAB49652

Genbank version no. AAB49652.1 GI:1888316

Genbank record update date: Jun. 23, 2010 09:22 AM

Cross-References

Scanlan, M. J.,et al Proc. Natl. Acad. Sci. U.S.A. 91 (12), 5657-5661 (1994)

Other information

Official Symbol: FAP

Other Aliases: DPPIV, FAPA

Other Designations: 170 kDa melanoma membrane-bound gelatinase; integral membrane serine protease; seprase

(83) DKK-1 (Dickkopf 1 homolog (Xenopus laevis)

Nucleotide

Genbank accession no. NM_012242

Genbank version no. NM_012242.2 GI:61676924

Genbank record update date: Sep. 30, 2012 01:48 PM

Polypeptide

Genbank accession no. NP_036374

Genbank version no. NP_036374.1 GI:7110719

Genbank record update date: Sep. 30, 2012 01:48 PM

Cross-References

Fedi P. et al J.Biol. Chem. 274 (27), 19465-19472 (1999)

Other Information

Official Symbol: DKK1

Other Aliases: UNQ492/PRO1008, DKK-1, SK

Other Designations: dickkopf related protein-1; dickkopf-1 like; dickkopf-like protein 1; dickkopf-related protein 1; hDkk-1

ANTIBODIES

Novartis: BHQ880 (Fulciniti M., et al Blood. 2009 Jul. 9; 114(2):371-379)

For example, see US20120052070A1 SEQ ID NOs: 100 and 108.

(84) CD52 (CD52 molecule)

Nucleotide

Genbank accession no. NM_001803

Genbank version no. NM_001803.2 GI:68342029

Genbank record update date: Sep. 30, 2012 01:48 PM

Polypeptide

Genbank accession no. NP_001794

Genbank version no. NP_001794.2 GI:68342030

Genbank record update date: Sep. 30, 2012 01:48 PM

Cross-References

Xia M. Q., et al Eur. J. Immunol. 21 (7), 1677-1684 (1991)

Other Information

Official Symbol: CD52

Other Aliases: CDW52

Other Designations: CAMPATH-1 antigen; CD52 antigen (CAMPATH-1 antigen); CDW52 antigen (CAMPATH-1 antigen); cambridge pathology 1 antigen; epididymal secretory protein E5; he5; human epididymis-specific protein 5

ANTIBODIES

Alemtuzumab (Campath)-Skoetz N., et al Cochrane Database Syst Rev. 2012 Feb. 15; 2:CD008078.
For example, see Drugbank Acc. No. DB00087 (BIOD00109, BTD00109)

(85) CS1- SLAMF7 (SLAM family member 7)

Nucleotide

Genbank accession no. NM_021181

Genbank version no. NM_021181.3 GI:1993571

Genbank record update date: Jun. 29, 2012 11:24 AM

Polypeptide

Genbank accession no. NP_067004

Genbank version no. NP_067004.3 GI:19923572

Genbank record update date: Jun. 29, 2012 11:24 AM

Cross-References

Boles K. S., et al Immunogenetics 52 (3-4), 302-307 (2001)

Other Information

Official Symbol: SLAMF7

Other Aliases: UNQ576/PRO1138, 19A, CD319, CRACC, CS1

Other Designations: 19A24 protein; CD2 subset 1; CD2-like receptor activating cytotoxic cells; CD2-like receptor-activating cytotoxic cells; membrane protein FOAP-12; novel LY9 (lymphocyte antigen 9) like protein; protein 19A

ANTIBODIES

BMS: elotuzumab/HuLuc63 (Benson D M., et al J Clin Oncol. 2012 Jun. 1; 30(16):2013-2015)
For example, see US20110206701 SEQ ID NOs: 9, 10, 11, 12, 13, 14, 15 and 16.

(86) Endoglin-ENG (Endoglin)

Nucleotide

Genbank accession no. AF035753

Genbank version no. AF035753.1 GI:3452260

Genbank record update date: Mar. 10, 2010 06:36 PM

Polypeptide

Genbank accession no. AAC32802

Genbank version no. AAC32802.1 GI:3452261

Genbank record update date: Mar. 10, 2010 06:36 PM

Cross-References

Rius C., et al Blood 92 (12), 4677-4690 (1998)

Official Symbol: ENG

Other Information

Other Aliases: RP11-228B15.2, CD105, END, HHT1, ORW, ORW1

Other Designations: CD105 antigen

(87) Annexin A1-ANXA1 (Annexin A1)

Nucleotide

Genbank accession no. X05908

Genbank version no. X05908.1 GI:34387

Genbank record update date: Feb. 02, 2011 10:02 AM

Polypeptide

Genbank accession no. CCA29338

Genbank version no. CCA29338.1 GI:34388

Genbank record update date: Feb. 02, 2011 10:02 AM

Cross-References

Wallner B. P.,et al Nature 320 (6057), 77-81 (1986)

Other Information

Official Symbol: ANXA1

Other Aliases: RP11-71A24.1, ANX1, LPC1

Other Designations: annexin I (lipocortin I); annexin-I; calpactin II; calpactin-2; chromobindin-9; lipocortin I; p35; phospholipase A2 inhibitory protein

(88) V-CAM (CD106)-VCAM1 (Vascular cell adhesion molecule 1)

Nucleotide

Genbank accession no. M60335

Genbank version no. M60335.1 GI:340193

Genbank record update date: Jun. 23, 2010 08:56 AM

Polypeptide

Genbank accession no. AAA61269

Genbank version no. AAA61269.1 GI:340194

Genbank record update date: Jun. 23, 2010 08:56 AM

Cross-References

Hession C., et al J. Biol. Chem. 266 (11), 6682-6685 (1991)

Other Information

Official Symbol VCAM1

Other Aliases: CD106, INCAM-100

Other Designations: CD106 antigen; vascular cell adhesion protein 1

Antibody Sequences
Anti-Integrin αvβ6
RHAB6.2
QVQLVQSGSELKKPGASVKISCKASGFAFTDSYMHWVRQAPGQGLEW
MGWIDPENGDTEYAPKFQGRFVFSLDTSVSTAYLQISSLKAEDTAVY
YCTRGTPTAVPNLRGDLQVLAQKVAGPYPFDYWGQGTLVTVSS
RHCB6.2
QVQLVQSGAEVKKPGASVKVSCKASGYTFIDSYMHWVRQAPGQRLEW
MGWIDPENGDTEYAPKFQGRVTITTDTSASTAYMELSSLRSEDTAVY
YCARGTPTAVPNLRGDLQVLAQKVAGPYPFDYWGQGTLVTVSS
RHF
QVQLVQSGAEVKKPGASVKVSCKASGFNFIDSYMHWVRQAPGQRLEW
MGWIDPENGDTEYAPKFQGRVTFTTDTSASTAYMELSSLRSEDTAVY
YCNEGTPTGPYYFDYWGQGTLVTVSS
RHFB6
QVQLVQSGAEVKKPGASVKVSCKASGFNFIDSYMHWVRQAPGQRLEW
MGWIDPENGDTEYAPKFQGRVTFTTDTSASTAYMELSSLRSEDTAVY
YCNEGTPTAVPNLRGDLQVLAQKVAGPYYFDYWGQGTLVTVSS
RHAY100bP
QVQLVQSGSELKKPGASVKISCKASGFAFTDSYMHWVRQAPGQGLEW
MGWIDPENGDTEYAPKFQGRFVFSLDTSVSTAYLQISSLKAEDTAVY
YCTRGTPTGPYPFDYWGQGTLVTVSS
RKF
ENVLTQSPGTLSLSPGERATLSCSASSSVSYMHWFQQKPGQAPRLLI
YSTSNLASGIPDRFSGSGSGTDFTLTISRLEPEDFAVYYCQQRSSYP
LTFGGGTKVEIK
RKFL36L50
ENVLTQSPGTLSLSPGERATLSCSASSSVSYMHWLQQKPGQAPRLLI
YLTSNLASGIPDRFSGSGSGTDFTLTISRLEPEDFAVYYCQQRSSYP
LTFGGGTKVEIK
RKC
EIVLTQSPGTLSLSPGERATLSCSASSSVSYMHWFQQKPGQAPRLLI
YSTSNLASGIPDRFSGSGSGTDFTLTISRLEPEDFAVYYCQQRSSYP
LTFGGGTKVEIK
Anti-CD33
CD33 Hum195 VH
QVQLVQSGAEVKKPGSSVKVSCKASGYTFTDYNMHWVRQAPGQGLEW
IGYIYPYNGGTGYNQKFKSKATITADESTNTAYMELSSLRSEDTAVY
YCARGRPAMDYWGQGTLVTVSS
CD33 Hum195 VK
DIQMTQSPSSLSASVGDRVTITCRASESVDNYGISFMNWFQQKPGKA
PKLLIYAASNQGSGVPSRFSGSGSGTDFTLTISSLQPDDFATYYCQQ
SKEVPWTFGQGTKVEIK
Anti-CD19
CD19 B4 resurfaced VH
QVQLVQPGAEVVKPGASVKLSCKTSGYTFTSNWMHWVKQRPGQGLEW
IGEIDPSDSYTNYNQNFKGKAKLTVDKSTSTAYMEVSSLRSDDTAVY
YCARGSNPYYYAMDYWGQGTSVTVSS
CD19 B4 resurfaced VK
EIVLTQSPAIMSASPGERVTMTCSASSGVNYMHWYQQKPGTSPRRWI
YDTSKLASGVPARFSGSGSGTSYSLTISSMEPEDAATYYCHQRGSYT
FGGGTKLEIK
Anti-Her2
Herceptin VH chain
EVQLVESGGGLVQPGGSLRLSCAASGFNIKDTYIHWVRQAPGKGLEW
VARIYPTNGYTRYADSVKGRFTISADTSKNTAYLQMNSLRAEDTAVY
YCSRWGGDGFYAMDYWGQGTLVTVSS
Herceptin VL chain
DIQMTQSPSSLSASVGDRVTITCRASQDVNTAVAWYQQKPGKAPKLL
IYSASFLYSGVPSRFSGSRSGTDFTLTISSLQPEDFATYYCQQHYTT
PPTFGQGTKVEIK
Anti-CD25
Simulect VK (also known as Basiliximab)
QIVSTQSPAIMSASPGEKVTMTCSASSSRSYMQWYQQKPGTSPKRWI
YDTSKLASGVPARFSGSGSGTSYSLTISSMEAEDAATYYCHQRSSYT
FGGGTKLEIK
Simulect VH
QLQQSGTVLARPGASVKMSCKASGYSFTRYWMHWIKQRPGQGLEWIG
AIYPGNSDTSYNQKFEGKAKLTAVTSASTAYMELSSLTHEDSAVYYC
SRDYGYYFDFWGQGTTLTVSS
Anti-PSMA
Deimmunised VH ′1
EVQLVQSGPEVKKPGATVKISCKTSGYTFTEYTIHWVKQAPGKGLEW
IGNINPNNGGTTYNQKFEDKATLTVDKSTDTAYMELSSLRSEDTAVY
YCAAGWNFDYWGQGTLLTVSS
Deimmunised VK ′1
DIQMTQSPSSLSTSVGDRVTLTCKASQDVGTAVDWYQQKPGPSPKLL
IYWASTRHTGIPSRFSGSGSGTDFTLTISSLQPEDFADYYCQQYNSY
PLTFGPGTKVDIK
Deimmunised VH1 ′5
EVKLVESGGGLVQPGGSMKLSCVASGFTFSNYWMNWVRQAPGKGLEW
VAEIRSQSNNFATHYAESVKGRVTISRDDSKSIVYLQMNNLRAEDTG
VYYCTRRWNNFWGQGTTVTVSS
Deimmunised VH2 ′5
EVKLVESGGGLVQPGGSLKLSCVASGFTFSNYWMNWVRQAPGKGLEW
VAEIRSQSNNFATHYAESVKGRVTISRDDSKSIVYLQMNNLRAEDTA
VYYCTRRWNNFWGQGTTVTVSS
Deimmunised VH3 ′5
EVQLVESGGGLVQPGGSLKLSCVASGFTFSNYWMNWVRQAPGKGLEW
VAEIRSQSNNFATHYAESVKGRVTISRDDSKSIVYLQMNNLRAEDTA
VYYCTRRWNNFWGQGTTVTVSS
Deimmunised VH4 ′5
EVQLVESGGGLVQPGGSLKLSCVASGFTFSNYWMNWVRQAPGKGLEW
VAEIRSQSNNFATHYAESVKGRFTISRDDSKSIVYLQMNNLRAEDTA
VYYCTRRWNNFWGQGTTVTVSS
Deimmunised VK1 ′5
NIVMTQFPSSMSASVGDRVTITCKASENVGTYVSWYQQKPDQSPKML
IYGASNRFTGVPDRFTGSGSATDFTLTISSLQTEDLADYYCGQSYTF
PYTFGQGTKLEMK
Deimmunised VK2 ′5
NIVMTQFPSSMSASVGDRVTITCKASENVGTYVSWYQQKPDQSPKML
IYGASNRFTGVPDRFSGSGSGTDFTLTISSLQAEDLADYYCGQSYTF
PYTFGQGTKLEIK
Deimmunised VK3 ′5
NIQMTQFPSAMSASVGDRVTITCKASENVGTYVSWYQQKPDQSPKML
IYGASNRFTGVPDRFSGSGSGTDFTLTISSLQAEDLADYYCGQSYTF
PYTFGQGTKLEIK
Deimmunised VK4 ′5
NIQMTQFPSAMSASVGDRVTITCKASENVGTYVSWYQQKPDQSPKML
IYGASNRFTGVPDRFSGSGSGTDFTLTISSLQAEDEADYYCGQSYTF
PYTFGQGTKLEIK
Deimmunised VK DI ′5
NIVMTQFPKSMSASAGERMTLTCKASENVGTYVSWYQQKPTQSPKML
IYGASNRFTGVPDRFSGSGSGTDFILTISSVQAEDLVDYYCGQSYTF
PYTFGGGTKLEMK
Deimmunised VH DI ′5
EVKLEESGGGLVQPGGSMKISCVASGFTFSNYWMNWVRQSPEKGLEW
VAEIRSQSNNFATHYAESVKGRVIISRDDSKSSVYLQMNSLRAEDTA
VYYCTRRWNNFWGQGTTVTVSS
Humanised RHA ′5
EVQLVESGGGLVQPGGSLKLSCAASGFTFSNYWMNWVRQASGKGLEW
VGEIRSQSNNFATHYAESVKGRFTISRDDSKNTAYLQMNSLKTEDTA
VYYCTRRWNNFWGQGTTVTVSS
Humanised RHB ′5
EVKLVESGGGLVQPGGSLKLSCAASGFTFSNYWMNWVRQASGKGLEW
VAEIRSQSNNFATHYAESVKGRVIISRDDSKNTVYLQMNSLRTEDTA
VYYCTRRWNNFWGQGTTVTVSS
Humanised RHC ′5
EVQLVESGGGLVQPGGSLKLSCAASGFTFSNYWMNWVRQASGKGLEW
VAEIRSQSNNFATHYAESVKGRVIISRDDSKNTVYLQMNSLRTEDTA
VYYCTRRWNNFWGQGTTVTVSS
Humanised RHD ′5
EVKLVESGGGLVQPGGSLKLSCAASGFTFSNYWMNWVRQASGKGLEW
VGEIRSQSNNFATHYAESVKGRVIISRDDSKNTVYLQMNSLRTEDTA
VYYCTRRWNNFWGQGTTVTVSS
Humanised RHE ′5
EVKLVESGGGLVQPGGSLKLSCAASGFTFSNYWMNWVRQASGKGLEW
VAEIRSQSNNFATHYAESVKGRFTISRDDSKNTVYLQMNSLRTEDTA
VYYCTRRWNNFWGQGTTVTVSS
Humanised RHF ′5
EVKLVESGGGLVQPGGSLKLSCAASGFTFSNYWMNWVRQASGKGLEW
VAEIRSQSNNFATHYAESVKGRVIISRDDSKNTAYLQMNSLRTEDTA
VYYCTRRWNNFWGQGTTVTVSS
Humanised RHG ′5
EVKLVESGGGLVQPGGSLKLSCAASGFTFSNYWMNWVRQASGKGLEW
VAEIRSQSNNFATHYAESVKGRVIISRDDSKNTAYLQMNSLRTEDTA
VYYCTRRWNNFWGQGTTVTVSS
Humanised RKA ′5
DIQMTQSPSSVSASVGDRVTITCKASENVGTYVSWYQQKPGTAPKLL
IYGASNRFTGVPSRFSGSGSATDFTLTINNLQPEDFATYYCGQSYTF
PYTFGQGTKVEIK
Humanised RKB ′5
DIQMTQSPSSVSASVGDRVTITCKASENVGTYVSWYQQKPGTAPKLL
IYGASNRFTGVPSRFSGSGSATDFTLTINNLQPEDFATYYCGQSYTF
PYTFGQGTKVEIK
Humanised RKC ′5
DIQMTQSPSSVSASVGDRVTITCKASENVGTYVSWYQQKPGTAPKML
IYGASNRFTGVPSRFSGSGSATDFTLTINNLQPEDFATYYCGQSYTF
PYTFGQGTKVEIK
Humanised RKD ′5
DIQMTQSPSSVSASVGDRVTITCKASENVGTYVSWYQQKPGTAPKML
IYGASNRFTGVPSRFSGSGSATDFTLTINNLQPEDFATYYCGQSYTF
PYTFGQGTKVEIK
Humanised RKE ′5
NIVMTQSPSSVSASVGDRVTITCKASENVGTYVSWYQQKPGTAPKLL
IYGASNRFTGVPDRFTGSGSATDFILTINNLQPEDFATYYCGQSYTF
PYTFGQGTKVEIK
Humanised RKF ′5
NIVMTQSPSSVSASVGDRVTITCKASENVGTYVSWYQQKPGTAPKML
IYGASNRFTGVPSRFSGSGSATDFILTINNLQPEDFATYYCGQSYTF
PYTFGQGTKVEIK
Humanised RKG ′5
NIVMTQSPSSVSASVGDRVTITCKASENVGTYVSWYQQKPGTAPKML
IYGASNRFTGVPDRFTGSGSATDFTLTINNLQPEDFATYYCGQSYTF
PYTFGQGTKVEIK

The parent antibody may also be a fusion protein comprising an albumin-binding peptide (ABP) sequence (Dennis et al. (2002) “Albumin Binding As A General Strategy For Improving The Pharmacokinetics Of Proteins” J Biol Chem. 277:35035-35043; WO 01/45746). Antibodies of the invention include fusion proteins with ABP sequences taught by: (i) Dennis et al (2002) J Biol Chem. 277:35035-35043 at Tables III and IV, page 35038; (ii) US 2004/0001827 at [0076]; and (iii) WO 01/45746 at pages 12-13, and all of which are incorporated herein by reference.

In one embodiment, the antibody has been raised to target specific the tumour related antigen αᵥβ₆.

The cell binding agent may be labelled, for example to aid detection or purification of the agent either prior to incorporation as a conjugate, or as part of the conjugate. The label may be a biotin label. In another embodiment, the cell binding agent may be labelled with a radioisotope.

Connection of Linker Unit to Ligand Unit

The Ligand unit is connected to the Linker unit through a disulfide bond.

In one embodiment, the connection between the Ligand unit and the Drug Linker is formed between a thiol group of a cysteine residue of the Ligand unit and a maleimide group of the Drug Linker unit.

The cysteine residues of the Ligand unit may be available for reaction with the functional group of the Linker unit to form a connection. In other embodiments, for example where the Ligand unit is an antibody, the thiol groups of the antibody may participate in interchain disulfide bonds. These interchain bonds may be converted to free thiol groups by e.g. treatment of the antibody with DTT prior to reaction with the functional group of the Linker unit.

In some embodiments, the cysteine residue is an introduced into the heavy or light chain of an antibody. Positions for cysteine insertion by substitution in antibody heavy or light chains include those described in Published U.S. Application No. 2007-0092940 and International Patent Publication WO2008070593, which are incorporated herein.

Methods of Treatment

The compounds of the present invention may be used in a method of therapy. Also provided is a method of treatment, comprising administering to a subject in need of treatment a therapeutically-effective amount of a conjugate of formula I. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g. drugs; surgery; and radiation therapy.

Pharmaceutical compositions according to the present invention, and for use in accordance with the present invention, may comprise, in addition to the active ingredient, i.e. a conjugate of formula I, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The Conjugates can be used to treat proliferative disease and autoimmune disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g., histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin.

Examples of autoimmune disease include the following: rheumatoid arthritis, autoimmune demyelinative diseases (e.g., multiple sclerosis, allergic encephalomyelitis), psoriatic arthritis, endocrine ophthalmopathy, uveoretinitis, systemic lupus erythematosus, myasthenia gravis, Graves' disease, glomerulonephritis, autoimmune hepatological disorder, inflammatory bowel disease (e.g., Crohn's disease), anaphylaxis, allergic reaction, Sjögren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), male and female autoimmune infertility, ankylosing spondolytis, ulcerative colitis, mixed connective tissue disease, polyarteritis nedosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the autoimmune disease is a disorder of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjögren's syndrome, Hashimoto's thyroiditis, Graves' disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes. In some embodiments, the autoimmunie disorder is a T cell-mediated immunological disorder.

In some embodiments, the amount of the Conjugate administered ranges from about 0.01 to about 10 mg/kg per dose. In some embodiments, the amount of the Conjugate administered ranges from about 0.01 to about 5 mg/kg per dose. In some embodiments, the amount of the Conjugate administerd ranges from about 0.05 to about 5 mg/kg per dose. In some embodiments, the amount of the Conjugate administerd ranges from about 0.1 to about 5 mg/kg per dose. In some embodiments, the amount of the Conjugate administered ranges from about 0.1 to about 4 mg/kg per dose. In some embodiments, the amount of the Conjugate administered ranges from about 0.05 to about 3 mg/kg per dose. In some embodiments, the amount of the Conjugate administered ranges from about 0.1 to about 3 mg/kg per dose. In some embodiments, the amount of the Conjugate administered ranges from about 0.1 to about 2 mg/kg per dose.

Drug Loading

The drug loading (p) is the average number of PBD drugs per cell binding agent, e.g. antibody. Where the compounds of the invention are bound to cysteines, drug loading may range from 1 to 8 drugs (D) per cell binding agent, i.e. where 1, 2, 3, 4, 5, 6, 7, and 8 drug moieties are covalently attached to the cell binding agent. Compositions of conjugates include collections of cell binding agents, e.g. antibodies, conjugated with a range of drugs, from 1 to 8. Where the compounds of the invention are bound to lysines, drug loading may range from 1 to 80 drugs (D) per cell binding agent, although an upper limit of 40, 20, 10 or 8 may be preferred. Compositions of conjugates include collections of cell binding agents, e.g. antibodies, conjugated with a range of drugs, from 1 to 80, 1 to 40, 1 to 20, 1 to 10 or 1 to 8.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, p may be limited by the number of attachment sites on the antibody. For example, an antibody may have only one or several cysteine thiol groups, or may have only one or several sufficiently reactive thiol groups through which a linker may be attached. Higher drug loading, e.g. p >5, may cause aggregation, insolubility, toxicity, or loss of cellular permeability of certain antibody-drug conjugates.

Typically, fewer than the theoretical maximum of drug moieties are conjugated to an antibody during a conjugation reaction. An antibody may contain, for example, many lysine residues that do not react with the Drug Linker. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of Drug Linker relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula, et al., 2008b Nature Biotech., 26(8):925-932; Dornan et al (2009) Blood 114(13):2721-2729; U.S. Pat. No. 7,521,541; U.S. Pat. No. 7,723,485; WO2009/052249). The engineered cysteine thiols may react with linker reagents or the drug-linker reagents of the present invention which have thiol-reactive, electrophilic groups such as maleimide or alpha-halo amides to form ADC with cysteine engineered antibodies and the PBD drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the invention include mixtures of antibody-drug conjugate compounds where the antibody has one or more PBD drug moieties and where the drug moieties may be attached to the antibody at various amino acid residues.

In one embodiment, the average number of dimer pyrrolobenzodiazepine groups per cell binding agent is in the range 1 to 20. In some embodiments the range is selected from 1 to 8, 2 to 8, 2 to 6, 2 to 4, and 4 to 8.

In some embodiments, there is one dimer pyrrolobenzodiazepine group per cell binding agent.

General Synthetic Routes

The synthesis of PBD compounds is extensively discussed in the following references, which discussions are incorporated herein by reference:

a) WO 00/12508 (pages 14 to 30);

b) WO 2005/023814 (pages 3 to 10);

c) WO 2004/043963 (pages 28 to 29); and

d) WO 2005/085251 (pages 30 to 39).

Synthesis Route

The Drug Linker compounds of the present invention may be synthesised according to the Examples.

Synthesis of Drug Conjugates

Conjugates can be prepared as previously described. Antibodies can be conjugated to the Drug Linker compound as described in Doronina et al., Nature Biotechnology, 2003, 21, 778-784). Briefly, antibodies (4-5 mg/mL) in PBS containing 50 mM sodium borate at pH 7.4 are reduced with tris(carboxyethyl)phosphine hydrochloride (TCEP) at 37° C. The progress of the reaction, which reduces interchain disulfides, is monitored by reaction with 5,5′-dithiobis(2-nitrobenzoic acid) and allowed to proceed until the desired level of thiols/mAb is achieved. The reduced antibody is then cooled to 0° C. and alkylated with 1.5 equivalents of maleimide drug-linker per antibody thiol. After 1 hour, the reaction is quenched by the addition of 5 equivalents of N-acetyl cysteine. Quenched drug-linker is removed by gel filtration over a PD-10 column. The ADC is then sterile-filtered through a 0.22 μm syringe filter. Protein concentration can be determined by spectral analysis at 280 nm and 329 nm, respectively, with correction for the contribution of drug absorbance at 280 nm. Size exclusion chromatography can be used to determine the extent of antibody aggregation, and RP-HPLC can be used to determine the levels of remaining NAC-quenched drug-linker.

Further Preferences

The following preferences may apply to all aspects of the invention as described above, or may relate to a single aspect. The preferences may be combined together in any combination.

In some embodiments, the C11 substituent may be in the following stereochemical arrangement relative to neighbouring groups:

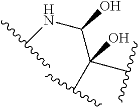

In other embodiments, the C11 substituent may be in the following stereochemical arrangement relative to neighbouring groups:

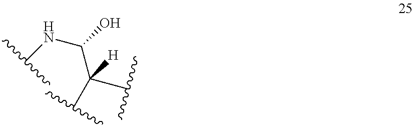

### EXAMPLES

Reaction progress was monitored by thin-layer chromatography (TLC) using Merck Kieselgel 60 F254 silica gel, with fluorescent indicator on aluminium plates. Visualisation of TLC was achieved with UV light or iodine vapour unless otherwise stated. Flash chromatography was performed using Merck Kieselgel 60 F254 silica gel. Extraction and chromatography solvents were bought and used without further purification from VWR, U.K. All chemicals were purchased from Aldrich.

Proton NMR chemical shift values were measured on the delta scale at 400 MHz using a Bruker AV400. The following abbreviations have been used: s, singlet; d, doublet; t, triplet; q, quartet; quin, quintet; m, multiplet; br, broad. Coupling constants are reported in Hz.

Column chromatography was performed on an Isolera (Biotage) automated system using normal phase SNAP cartridges.

The LC/MS Conditions were as Follow:

LCMS data were obtained using a Shimadzu Nexera series LC/MS with a Shimadzu LCMS-2020 quadrupole MS, with Electrospray ionisation. Mobile phase A-0.1% formic acid in water. Mobile phase B-0.1% formic acid in acetonitrile.

Short run gradient: initial composition was 5% B held over 0.25 min, then increase from 5% B to 100% B over a 2 min period. The composition was held for 0.50 min at 100% B, then returned to 5% B in 0.05 minutes and hold there for 0.05 min. Total gradient run time equals 3 min. Flow rate 0.8 mL/min. Wavelength detection range: 190 to 800 nm. Oven temperature: 50° C. Column: Waters Acquity UPLC BEH Shield RP18 1.7 μm 2.1×50 mm.

Long run gradient: initial composition 5% B held over 1 min, then increase from 5% B to 100% B over a 9 min period. The composition was held for 2 min at 100% B, then returned to 5% B in 0.10 minutes and hold there for 3 min. Total gradient run time equals 15 min. Flow rate 0.6 mL/min. Wavelength detection range: 190 to 800 nm. Oven temperature: 50° C. Column: ACE Excel 2 C18-AR, 2 μ, 3.0×100 mm.

### Example 1

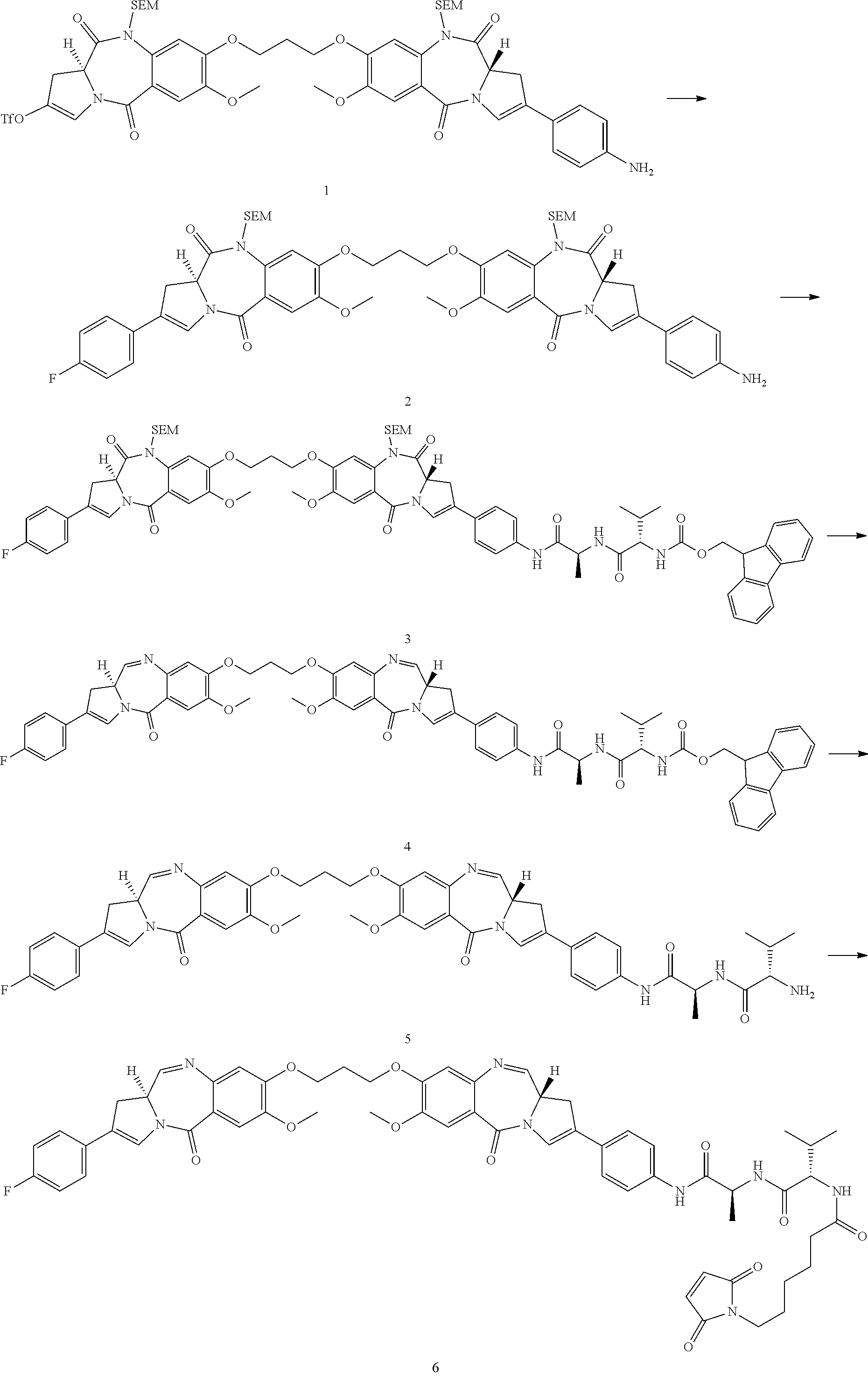

Compound 1 is compound 13 of WO 2014/057073.

(a) (S)-2-(4-aminophenyl)-8-(3-(((S)-2-(4-fluorophenyl)-7-methoxy-5,11-dioxo-10-((2-(trimethylsily)ethoxy)methyl)-5,10,11,11a-tetrahydro-1H-benzo[e]pyrrolo[1,2-a][1,4]diazepin-8-yl)oxy)propoxy)-7-methoxy-10-((2-(trimethylsilyl)ethoxy)methyl)-1,11a-dihydro-5H-benzo[e]pyrrolo[1,2-a][1,4]diazepine-5,11(10H)-dione (2) Pd(PPh₃)₄ (14 mg, 0.012 mmol) was added to a stirred mixture of the aniline-triflate 1 (666 mg, 0.62 mmol), boronic ester (167 mg, 0.75 mmol) and Na₂CO₃ (306 mg, 2.8 mmol) in a 2:1:1 mixture of toluene/MeOH/H₂O (20 mL). The reaction mixture was allowed to stir at 30° C. under a nitrogen atmosphere for 2 hours after which time all of triflate 1 has reacted. The reaction mixture was then evaporated to dryness before the residue was taken up in CH₂Cl₂ (50 mL) and washed with H₂O (2×50 mL), brine (50 mL), dried (MgSO₄), filtered and evaporated under reduced pressure to provide the crude product. Purification by flash chromatography (gradient elution: 1:1 v/v Hexane/EtOAc to 100% EtOAc) afforded product 2 as a yellow foam (522.4 mg, 82%). LC/MS 2.04 min (ES+) m/z (relative intensity) 1006.55 ([M+H]⁺., 100%).

(b) (9H-fluoren-9-yl)methyl ((S)-1-(((S)-1-((4-((S)-8-(3-(((S)-2-(4-fluorophenyl)-7-methoxy-5,11-dioxo-10-((2-(trimethylsilyl)ethoxy)methyl)-5,10,11,11a-tetrahydro-1H- benzo[e]pyrrolo[1,2-a][1,4]diazepin-8-yl)oxy)propoxy)-7-methoxy-5,11-dioxo-10-((2-(trimethylsily)ethoxy)methyl)-5,10,11,11a-tetrahydro-1H-benzo[e]pyrrolo[1,2-a][1,4]diazepin-2-yl)phenyl)amino)-1-oxopropan-2-yl)amino)-3-methyl-1-oxobutan-2-yl)carbamate (3)

To a solution of 2 (522.4 mg, 0.52 mmol) in dry CH₂C₁₂ (20 mL) was added the protected peptide (255 g, 0.62 mmol) and EEDQ (154 mg, 0.62 mmol). The mixture was stirred at room temperature until completion (16 hours). The reaction mixture was then washed with H₂O (2×25 mL), brine (25 mL), dried (MgSO₄), filtered and evaporated under reduced pressure to provide the crude product 3. Purification by flash chromatography (gradient elution: 100% CHCl₃ to 93/7 CHCl_{3/}MeOH) afforded product 3 as a yellow foam (655.9 mg, 90%). LC/MS 2.19 min (ES+) m/z (relative intensity) 1398.85 ([M+H]⁺., 40%); 1432.35 ([M+Na])⁺., 40%).

(c) (9H-fluoren-9-yl)methyl ((S)-1-(((S)-1-((4-((S)-8-(3-(((S)-2-(4-fluorophenyl)-7-methoxy-5-oxo-5,11a-dihydro-1H-benzo[e]pyrrolo[1,2-a][1,4]diazepin-8-yl)oxy)propoxy)-7-methoxy-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)carbamate (4)

A solution of Super-Hydride® (1.17 mL, 1M in THF) was added dropwise to a solution of SEM-dilactam 3 (655.9 mg, 0.47 mmol) in dry THF (3 mL) at −78° C. under an argon atmosphere. The addition was completed over 5 minutes in order to maintain the internal temperature of the reaction mixture constant. After 40 minutes, an aliquot was quenched with water for LC/MS analysis, which revealed that the reaction was complete. Water (20 mL) was added to the reaction mixture and the cold bath was removed. The organic layer was extracted with CH₂Cl₂ (3×50 mL) and the combined organics were washed with brine (100 mL), dried with MgSO_{4,} filtered and the solvent removed by rotary evaporation under reduced pressure. The crude product was dissolved in MeOH (9 mL), CH₂Cl₂ (3 mL), water (1 mL) and enough silica gel to form a thick stirring suspension. After 5 days, the suspension was filtered through a sintered funnel and washed with CH₂Cl₂/MeOH (9:1) (100 mL) until the elution of the product was complete. The organic layer was washed with brine (2×50 mL), dried with MgSO_{4,} filtered and the solvent removed by rotary evaporation under reduced pressure. Purification by silica gel column chromatography (isolera, CHCl₃/MeOH 98:2 to 80:20) afforded product 4 as a yellow solid (461.9 mg, 89%). LC/MS 1.74 min (ES+) m/z (relative intensity) 1106.40 ([M+H]⁺., 70%).

(d) (S)-2-amino-N-((S)-1-((4-((S)-8-(3-(((S)-2-(4-fluorophenyl)-7-methoxy-5-oxo-5,11a-dihydro-1H-benzo[e]pyrrolo[1,2-a][1,4]diazepin-8-yl)oxy)propoxy)-7-methoxy-5-oxo-5,11a-dihydro-1H-benzo[e]pyrrolo[1,2-a][1,4]diazepin-2-yl)phenyl)amino)-1-oxopropan-2-yl)-3-methylbutanamide (5)

Excess piperidine was added (0.15 mL) to a solution of PBD 4 (461.9 mg, 0.41 mmol) in DMF (3 mL). The mixture was allowed to stir at room temperature for 20 minutes, at which point the reaction had gone to completion (as monitored by LC/MS). The reaction mixture was diluted with CH₂Cl₂ (50 mL) and the organic phase was washed with H₂O (2×50 mL) until complete piperidine removal. The organic phase was dried over MgSO_{4,} filtered and excess solvent removed by rotary evaporation under reduced pressure to afford crude product 5 which was used as such in the next step. LC/MS 1.16 min (ES+) m/z (relative intensity) 884.5 ([M+H]⁺., 10%).

(e) 6-(2,5-dioxo-2,5-dihydro-1H-pyrrol-1-yl)-N-((S)-1-(((S)-1-((4-((S)-8-(3-(((S)-2-(4-fluoro-phenyl)-7-methoxy-5-oxo-5,11a-dihydro-1H-benzo[e]pyrrolo[1,2-a][1,4]diazepin-8-yl)oxy)propoxy)-7-methoxy-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)hexanamide (6) EDCl hydrochloride (80 mg, 0.41 mmol) and 6-maleimidohexanoic acid (88 mg, 0.41 mmol) were added to a solution of crude 5 in dry CH₂Cl₂ (5 mL) under an argon atmosphere. Stirring was maintained until the reaction was complete (4 hours). The reaction was diluted with CH₂Cl₂ (50 mL) and the organic phase was washed with H₂O (2×50 mL) and brine before being dried over MgSO_{4,} filtered and excess solvent removed by rotary evaporation under reduced pressure by rotary evaporation under reduced pressure. The product was purified by silica gel chromatography column (100% CHCl₃ to 95:5 CHCl₃/MeOH) affording 6 (125.8 mg, %). LC/MS 1.52 min (ES+) m/z (relative intensity) 1077.35 ([M+H]⁺., 20%). LC/MS (long run) 6.99 min (ES+) m/z (relative intensity) 1077.80 ([M+H]³⁰., 50%).

### Example 2

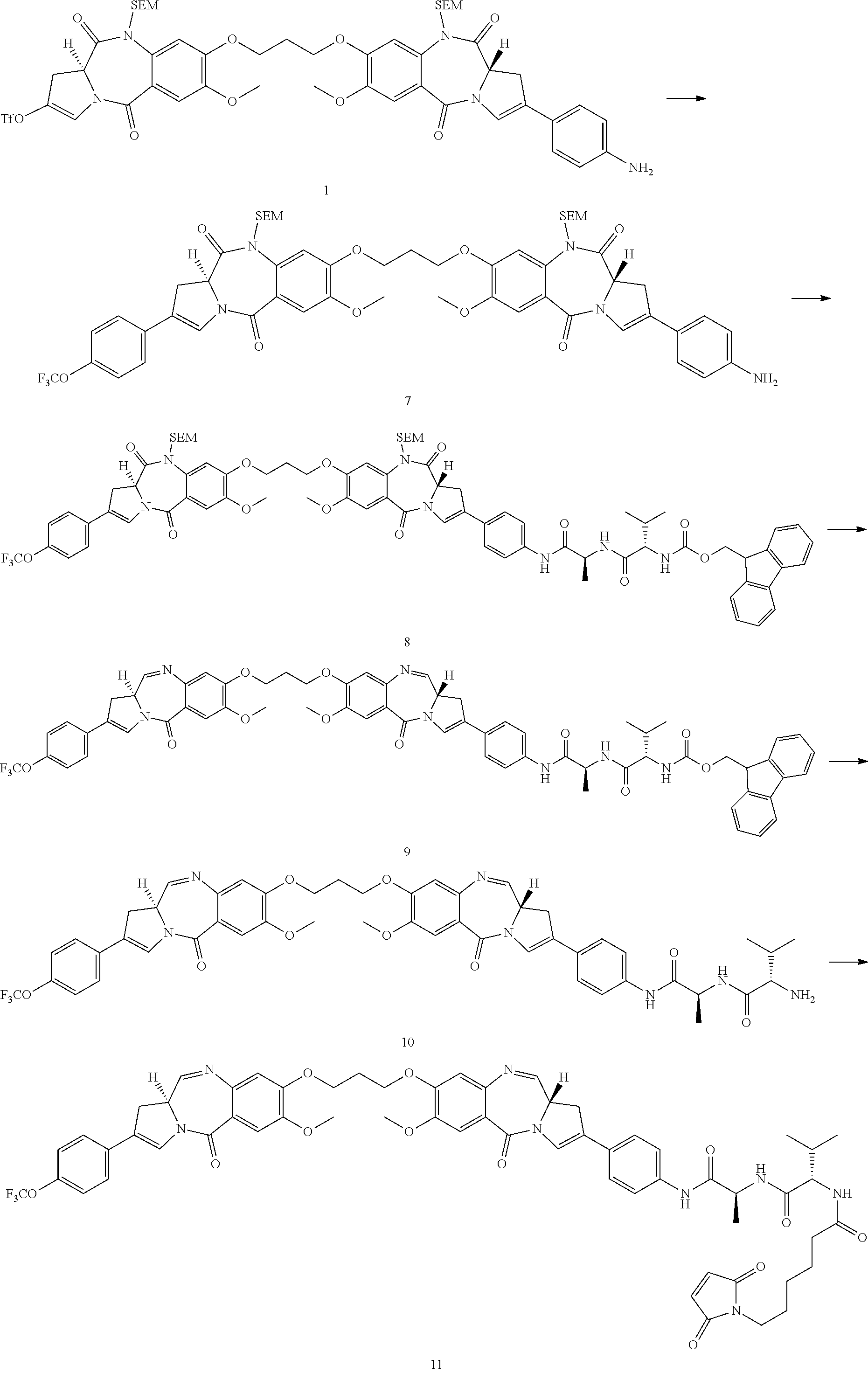

(a) (S)-2-(4-aminophenyl)-8-(3-(((S)-2-(4-(trifluoromethoxy)phenyl)-7-methoxy-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)propoxy)-7-methoxy-10-((2-(trimethylsilyl)ethoxy)methyl)-1,11a-dihydro-5H-benzo[e]pyrrolo[1,2-a][1,4]diazepine-5,11(10H)-dione (7) Pd(PPh₃)₄ (14 mg, 0.012 mmol) was added to a stirred mixture of the aniline-triflate 1 (666 mg, 0.62 mmol), boronic acid (155 mg, 0.75 mmol) and Na₂CO₃ (306 mg, 2.8 mmol) in a 2:1:1 mixture of toluene/MeOH/H₂O (20 mL). The reaction mixture was allowed to stir at 30° C. under a nitrogen atmosphere for 2 hours after which time all of triflate 1 has reacted. The reaction mixture was then evaporated to dryness before the residue was taken up in CH₂Cl₂ (50 mL) and washed with H₂O (2×50 mL), brine (50 mL), dried (MgSO₄), filtered and evaporated under reduced pressure to provide the crude product. Purification by flash chromatography (gradient elution: 1:1 v/v Hexane/EtOAc to 100% EtOAc) afforded product 7 as a yellow foam (501.1 mg, 74%). LC/MS 2.10 min (ES+) m/z (relative intensity) 1072.35 ([M+]⁺., 15%).

(b) (9H-fluoren-9-yl)methyl ((S)-1-(((S)-1-((4-((S)-8-(3-(((S)-2-(4-(trifluoromethoxy)phenyl)-7-methoxy-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)propoxy)-7-methoxy-5,11-dioxo-10-((2-(trimethylsily)ethoxy)methyl)-5,10,11,11a-tetrahydro-1H-benzo[e]pyrrolo[1,2-a][1,4]diazepin-2-yl)phenyl)amino)-1-oxopropan-2-yl)amino)-3-methyl-1-oxobutan-2-yl)carbamate (8)

To a solution of 7 (501.1 mg, 0.46 mmol) in dry CH₂Cl₂ (5 mL) was added the protected peptide (210 mg, 0.51 mmol) and EEDQ (127 mg, 0.51 mmol). The mixture was stirred at room temperature until completion (16 hours). The reaction mixture was then washed with H₂O (2×25 mL), brine (25 mL), dried (MgSO₄), filtered and evaporated under reduced pressure to provide the crude product. Purification by flash chromatography (gradient elution: 100% CHCl₃ to 93/7 CHCl₃/MeOH) afforded product 8 as a yellow foam (563 mg, 82%). LC/MS 2.23 min (ES+) m/z (relative intensity) 1465.30 ([M+H]⁺., 25%).

(c) (9H-fluoren-9-yl)methyl ((S)-1-(((S)-1-((4-((S)-8-(3-(((S)-2-(4-(trifluoromethoxy)phenyl)-7-methoxy-5-oxo-5,11a-dihydro-1H-benzo[e]pyrrolo[1,2-a][1,4]diazepin-8-yl)oxy)propoxy)-7-methoxy-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)carbamate (9) A solution of Super-Hydride® (0.96 mL, 1M in THF) was added dropwise to a solution of SEM-dilactam 8 (563 mg, 0.38 mmol) in dry THF (3 mL) at −78° C. under an argon atmosphere. The addition was completed over 5 minutes in order to maintain the internal temperature of the reaction mixture constant. After 40 minutes, an aliquot was quenched with water for LC/MS analysis, which revealed that the reaction was complete. Water (20 mL) was added to the reaction mixture and the cold bath was removed. The organic layer was extracted with CH₂Cl₂ (3×50 mL) and the combined organics were washed with brine (100 mL), dried with MgSO_{4,} filtered and the solvent removed by rotary evaporation under reduced pressure. The crude product was dissolved in MeOH (9 mL), CH₂Cl₂ (3 mL), water (1 mL) and enough silica gel to form a thick stirring suspension. After 5 days, the suspension was filtered through a sintered funnel and washed with CH₂Cl₂/MeOH (9:1) (100 mL) until the elution of the product was complete. The organic layer was washed with brine (2×50 mL), dried with MgSO_{4,} filtered and the solvent removed by rotary evaporation under reduced pressure. Purification by silica gel column chromatography (isolera, CHCl₃/MeOH 98:2 to 80:20) afforded product 9 as a yellow solid (428 mg, 95%). LC/MS 1.83 min (ES+) m/z (relative intensity) 1172.25 ([M+H]⁺., 80%).

(d) (S)-2-amino-N-((S)-1-((4-((S)-8-(3-(((S)-2-(4-(trifluoromethoxy)phenyl)-7-methoxy-5-oxo-5,11a-dihydro-1H-benzo[e]pyrrolo[1,2-a][1,4]diazepin-8-yl)oxy)propoxy)-7-methoxy-5-oxo-5,11a-dihydro-1H-benzo[e]pyrrolo[1,2-a][1,4]diazepin-2-yl)phenyl)amino)-1-oxopropan-2-yl)-3-methylbutanamide (10)

Excess piperidine was added (0.15 mL) to a solution of PBD 9 (428 mg, 0.36 mmol) in DMF (3 mL). The mixture was allowed to stir at room temperature for 20 minutes, at which point the reaction had gone to completion (as monitored by LC/MS). The reaction mixture was diluted with CH₂Cl₂ (50 mL) and the organic phase was washed with H₂O (2×50 mL) until complete piperidine removal. The organic phase was dried over MgSO_{4,} filtered and excess solvent removed by rotary evaporation under reduced pressure to afford crude product 10 which was used as such in the next step.: LC/MS 1.24 min (ES+) m/z (relative intensity) 950.35 ([M+H]⁺., 10%).

(e) 6-(2,5-dioxo-2,5-dihydro-1H-pyrrol-1-yl)-N-((S)-1-(((S)-1-((4-((S)-8-(3-(((S)-2-(4-(trifluoromethoxy)phenyl)-7-methoxy-5-oxo-5,11a-dihydro-1H-benzo[e]pyrrolo[1,2-a][1,4]diazepin-8-yl)oxy)propoxy)-7-methoxy-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)hexanamide (11)

EDCl hydrochloride (70 mg, 0.36 mmol) and 6-maleimidohexanoic acid (77 mg, 0.36 mmol) were added to a solution of crude 10 in dry CH₂Cl₂ (5 mL) under an argon atmosphere. Stirring was maintained until the reaction was complete (4 hours). The reaction was diluted with CH₂Cl₂ (50 mL) and the organic phase was washed with H₂O (2×50 mL) and brine before being dried over MgSO_{4,} filtered and excess solvent removed by rotary evaporation under reduced pressure by rotary evaporation under reduced pressure. The product was purified by silica gel chromatography column (100% CHCl₃ to 95:5 CHCl₃/MeOH) affording 11 (99 mg, 23%). LC/MS 1.61 min (ES+) m/z (relative intensity) 1143.25 ([M+H]⁺., 80%). LC/MS (long run) 7.46 min (ES+) m/z (relative intensity) 1143.25 ([M+H]⁺., 50%).

### Example 3

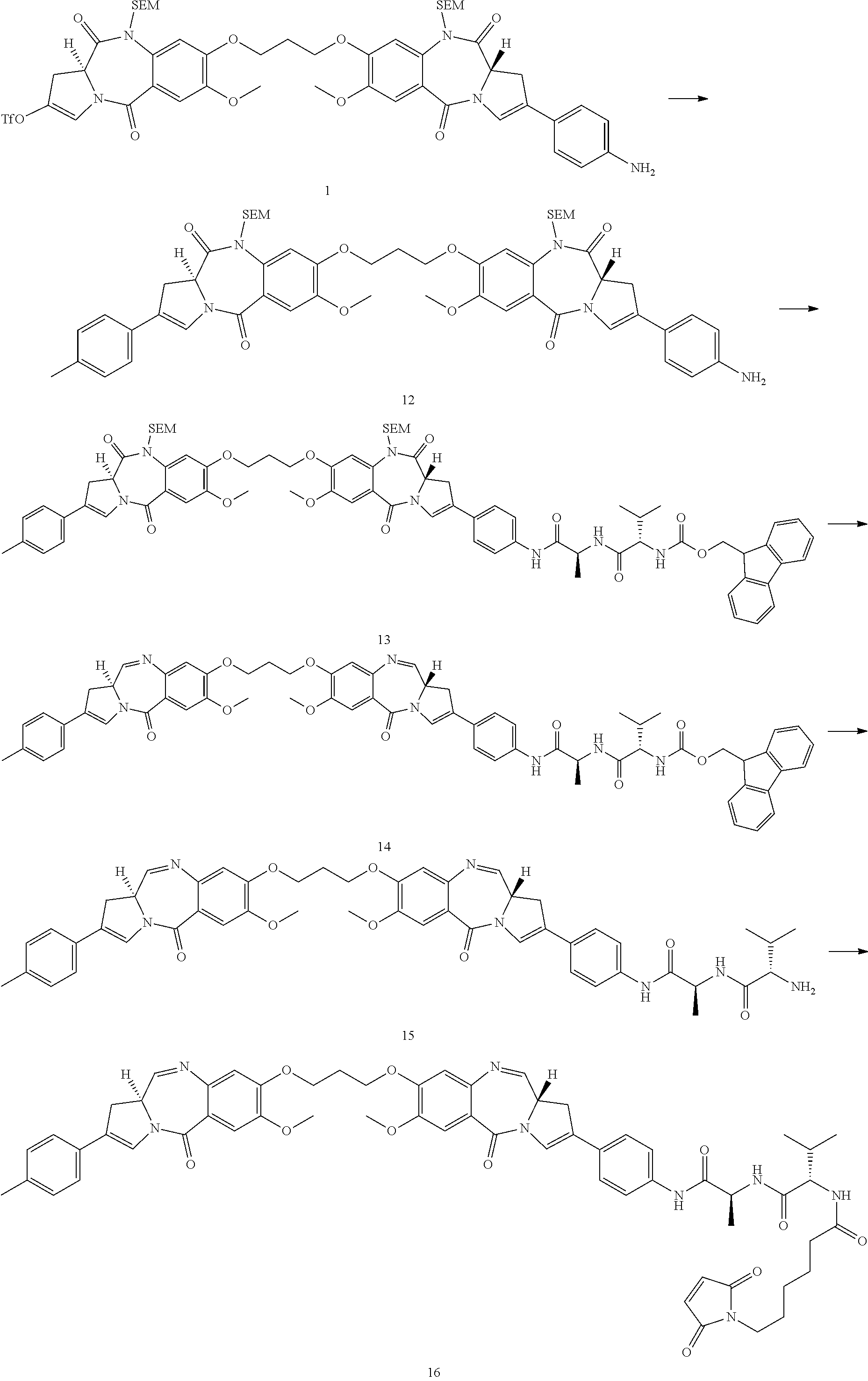

(a) (S)-2-(4-aminophenyl)-7-methoxy-8-(3-(((S)-7-methoxy-5,11-dioxo-2-(p-tolyl)-10-((2-(trimethylsily)ethoxy)methyl)-5,10,11,11a-tetrahydro-1H-benzo[e]pyrrolo[1,2-a][1,4diazepin-8-yl)oxy)propoxy)-10-((2-(trimethylsilyl)ethoxy)methyl)-1,11a-dihydro-5H-benzo[e]pyrrolo[1,2-a][1,4]diazepine-5,11(10H)-dione (12)

Pd(PPh₃)₄ (22 mg, 0.018 mmol) was added to a stirred mixture of the aniline-triflate 1 (1 g, 0.94 mmol), boronic ester (246 mg, 1.1 mmol) and Na₂CO₃ (459 mg, 4.3 mmol) in a 2:1:1 mixture of toluene/MeOH/H₂O (20 mL). The reaction mixture was allowed to stir at 30° C. under a nitrogen atmosphere for 2 hours after which time all of triflate 1 has reacted. The reaction mixture was then evaporated to dryness before the residue was taken up in CH₂Cl₂ (50 mL) and washed with H₂O (2×50 mL), brine (50 mL), dried (MgSO₄), filtered and evaporated under reduced pressure to provide the crude product. Purification by flash chromatography (gradient elution: 1:1 v/v Hexane/EtOAc to 100% EtOAc) afforded product 12 as a yellow foam (803.8 mg, 85%). LC/MS 2.07 min (ES+) m/z (relative intensity) 1002.35 ([M+]⁺., 20%).

(b) (9H-fluoren-9-yl)methyl ((S)-1-(((S)-1-((4-((S)-7-methoxy-8-(3-(((S)-7-methoxy-5,11-dioxo-2-(p-tolyl)-10-((2-(trimethylsilyl)ethoxy)methyl)-5,10,11,11a-tetrahydro-1H-benzo[e]pyrrolo[1,2-a][1,4]diazepin-8-yl)oxy)propoxy)-5,11-dioxo-10-((2-(trimethylsily)ethoxy)methyl)-5,10,11,11a-tetrahydro-1H-benzo[e]pyrrolo[1,2-a][1,4]diazepin-2-yl)phenyl)amino)-1-oxopropan-2-yl)amino)-3-methyl-1-oxobutan-2-yl)carbamate (13)

To a solution of 12 (803.8 mg, 0.80 mmol) in dry CH₂Cl₂ (25 mL) was added the protected peptide (361 mg, 0.88 mmol) and EEDQ (206 mg, 0.88 mmol). The mixture was stirred at room temperature until completion (16 hours). The reaction mixture was then washed with H₂O (2×50 mL), brine (50 mL), dried (MgSO₄), filtered and evaporated under reduced pressure to provide the crude product. Purification by flash chromatography (gradient elution: 100% CHCl3 to 93/7 CHCl₃/MeOH) afforded product 13 as a yellow foam (852.4 mg, 76%). LC/MS 2.20 min (ES+) m/z (relative intensity) 1395.35 ([M+H]⁺., 85%).

(c) (9H-fluoren-9-yl)methyl ((S)-1-(((S)-1-((4-((S)-7-methoxy-8-(3-(((S)-7-methoxy-5-oxo-2- (p-tolyl)-5,11a-dihydro-1H-benzo[e]pyrrolo[1,2-a][1,4]diazepin-8-yl)oxy)propoxy)-5-oxo- 5,11a-dihydro-1H-benzo[e]pyrrolo[1,2-a][1,4]diazepin-2-yl)phenyl)amino)-1-oxopropan-2- yl)amino)-3-methyl-1-oxobutan-2-yl)carbamate (14) A solution of Super-Hydride® (01.52 mL, 1M in THF) was added dropwise to a solution of SEM-dilactam 13 (852.4 mg, 0.61 mmol) in dry THF (5 mL) at −78° C. under an argon atmosphere. The addition was completed over 5 minutes in order to maintain the internal temperature of the reaction mixture constant. After 40 minutes, an aliquot was quenched with water for LC/MS analysis, which revealed that the reaction was complete. Water (20 mL) was added to the reaction mixture and the cold bath was removed. The organic layer was extracted with CH₂Cl₂ (3 ×50 mL) and the combined organics were washed with brine (100 mL), dried with MgSO_{4,} filtered and the solvent removed by rotary evaporation under reduced pressure. The crude product was dissolved in MeOH (9 mL), CH₂Cl₂ (3 mL), water (1 mL) and enough silica gel to form a thick stirring suspension. After 5 days, the suspension was filtered through a sintered funnel and washed with CH₂CL₂/MeOH (9:1) (100 mL) until the elution of the product was complete. The organic layer was washed with brine (2×50 mL), dried with MgSO_{4,} filtered and the solvent removed by rotary evaporation under reduced pressure. Purification by silica gel column chromatography (isolera, CHCl₃/MeOH 98:2 to 80:20) afforded product 14 as a yellow solid (672.1 mg, 100%). LC/MS 1.77min (ES+) m/z (relative intensity) 1102.40 ([M+H]³⁰ ., 10%).

(d) (S)-2-amino-N-((S)-1-((4-((S)-7-methoxy-8-(3-(((S)-7-methoxy-5-oxo-2-(p-tolyl)-5,11a-dihydro-1H-benzo[e]pyrrolo[1,2-a][1,4]diazepin-8-yl)oxy)propoxy)-5-oxo-5,11a-dihydro-1H- benzo[e]pyrrolo[1,2-a][1,4]diazepin-2-yl)phenyl)amino)-1-oxopropan-2-yl)-3- methylbutanamide (15)

Excess piperidine was added (0.25 mL) to a solution of PBD 14 (672.1 mg, 0.61 mmol) in DMF (3 mL). The mixture was allowed to stir at room temperature for 20 minutes, at which point the reaction had gone to completion (as monitored by LC/MS). The reaction mixture was diluted with CH₂Cl₂ (50 mL) and the organic phase was washed with H₂O (2 x 50 mL) until complete piperidine removal. The organic phase was dried over MgSO_{4,} filtered and excess solvent removed by rotary evaporation under reduced pressure to afford crude product 15 which was used as such in the next step. LC/MS 1.19 min (ES+) m/z (relative intensity) 880.40 ([M+H]⁺., 20%).

(e) 6-(2,5-dioxo-2,5-dihydro-1H-pyrrol-1-yl)-N-((S)-1-(((S)-1-((4-((S)-7-methoxy-8-(3-(((S)-7-methoxy-5-oxo-2-(p-tolyl)-5,11a-dihydro-1H-benzo[e]pyrrolo[1,2-a][1,4]diazepin-8-yl)oxy)propoxy)-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)hexanamide (16) EDCl hydrochloride (120 mg, 0.62 mmol) and 6-maleimidohexanoic acid (132 mg, 0.36 mmol) were added to a solution of crude 15 in dry CH₂Cl₂ (5 mL) under an argon atmosphere. Stirring was maintained until the reaction was complete (4 hours). The reaction was diluted with CH₂Cl₂ (50 mL) and the organic phase was washed with H₂O (2 x 50 mL) and brine before being dried over MgSO_{4,} filtered and excess solvent removed by rotary evaporation under reduced pressure by rotary evaporation under reduced pressure. The product was purified by silica gel chromatography column (100% CHCl₃ to 95:5 CHCl₃/MeOH) affording 16 (131.8 mg, 20%). LC/MS 1.55 min (ES+) m/z (relative intensity) 1073.50 ([M+H]⁺., 70%). LC/MS (long run) 7.19 min (ES+) m/z (relative intensity) 1073.40 ([M+H]⁺., 80%).

### Example 4-Conjugation

ConjA, Tratuzumab-6

A 50 mM solution of tris(2-carboxyethyl)phosphine hydrochloride (TCEP) in phosphate-buffered saline pH 7.4 (PBS) was added (50 molar equivalent/antibody, 18.3 micromoles, 183.3 μL) to a 22 mL solution of antibody, Trastuzumab (55 mg, 367 nanomoles) in reduction buffer containing PBS and 1 mM ethylenediaminetetraacetic acid (EDTA) and a final antibody concentration of 2.5 mg/mL. The reduction mixture was heated at +37° C. for 3 hours (or until full reduction is observed by UHPLC) in an incubator with gentle (135 rpm) shaking. After cooling down to room temperature, the reduced antibody was buffer exchanged, via spin filter centrifugation, into a reoxidation buffer containing PBS pH 7.4 and 1 mM EDTA to remove all the excess reducing agent. A 50 mM solution of dehydroascorbic acid (DHAA, 13 molar equivalent/antibody, 4.8 micromoles, 95.33 μL) in DMSO was added and the reoxidation mixture was allowed to react for 16 hours at room temperature with gentle (<150 rpm) shaking at an antibody concentration of 2 mg/mL (or more DHAA added and reaction left for longer until full reoxidation of the cysteine thiols to reform the inter-chain cysteine disulfides is observed by UHPLC). The reoxidation mixture was then sterile-filtered and diluted in a conjugation buffer containing PBS pH 7.4, 1 mM EDTA for a final antibody concentration of 1.0-1.5 mg/mL. Compound 6 was added as a solution in propylene glycol (20 molar equivalent/antibody, 2.0 micromole, in 0.8 mL propylene glycol) to 9 mL of this reoxidised antibody solution (15 mg, 100 nanomoles), followed by DMSO and further addition of propylene glycol for a 10% (v/v) final DMSO concentration and 40-50% (v/v) final propylene glycol concentration to solubilise the payload. The solution was mixed for 5 hours at room temperature, after which the conjugation was quenched by addition of N-acetyl-L-cysteine (8 micromoles, 80 μL at 100 mM). The conjugation mixture was purified by spin filtration using a 15 mL Amicon Ultracell 50 kDa MWCO spin filter, then further purified by tangential flow filtration (TFF) to >50 diavolumes using a Spectrum Labs KrosFlo Research Ili system with a 30 kDa MWCO, 115 cm² surface area hollow fibre filter module at 50 mL/min in PBS with a transmembrane pressure (TMP) of 0.5-1.0 bar, and sterile-filtered and analysed. UHPLC analysis on a Shimadzu Prominence system using a Phenomenex Aeris 3.6u XB-C18 150 mm×2.1 mm column eluting with a gradient of water and acetonitrile on a reduced sample of ConjA (Tratuzumab-6) at 280 nm and 330 nm (Compound 6 specific) shows unconjugated light chains and a mixture of unconjugated heavy chains and heavy chains attached to a single molecule of Compound 6, consistent with a drug-per-antibody ratio (DAR) of 1.62 molecules of Compound 6 per antibody.

UHPLC analysis on a Shimadzu Prominence system using a Tosoh Bioscience TSKgel G3000SWXL 5 μm 7.8×300 mm column (with a 7 μm 6.0×40 mm guard column) eluting with sterile-filtered SEC buffer containing 200 mM potassium phosphate pH 6.95, 250 mM potassium chloride and 10% isopropanol (v/v) on a sample of ConjA (Tratuzumab-6) at 280 nm shows a monomer purity of 95%. UHPLC SEC analysis gives a concentration of final ConjA (Tratuzumab-6) at 0.58 mg/mL in 18.0 mL, obtained mass of ConjA (Tratuzumab-6) is 10.5 mg (70% yield).

ConjB, Tratuzumab-11

A 50 mM solution of tris(2-carboxyethyl)phosphine hydrochloride (TCEP) in phosphate-buffered saline pH 7.4 (PBS) was added (50 molar equivalent/antibody, 18.3 micromoles, 183.3 μL) to a 22 mL solution of antibody (55 mg, 367 nanomoles) in reduction buffer containing PBS and 1 mM ethylenediaminetetraacetic acid (EDTA) and a final antibody concentration of 2.5 mg/mL. The reduction mixture was heated at +37 ° C. for 3 hours (or until full reduction is observed by UHPLC) in an incubator with gentle (135 rpm) shaking. After cooling down to room temperature, the reduced antibody was buffer exchanged, via spin filter centrifugation, into a reoxidation buffer containing PBS pH 7.4 and 1 mM EDTA to remove all the excess reducing agent. A 50 mM solution of dehydroascorbic acid (DHAA, 13 molar equivalent/antibody, 4.8 micromoles, 95.33 μL) in DMSO was added and the reoxidation mixture was allowed to react for 16 hours at room temperature with gentle (<150 rpm) shaking at an antibody concentration of 2 mg/mL (or more DHAA added and reaction left for longer until full reoxidation of the cysteine thiols to reform the inter-chain cysteine disulfides is observed by UHPLC). The reoxidation mixture was then sterile-filtered and diluted in a conjugation buffer containing PBS pH 7.4, 1 mM EDTA for a final antibody concentration of 1.0-1.5 mg/mL. Compound 11 was added as a solution in propylene glycol (20 molar equivalent/antibody, 2.0 micromole, in 0.8 mL propylene glycol) to 9 mL of this reoxidised antibody solution (15 mg, 100 nanomoles), followed by DMSO and further addition of propylene glycol for a 10% (v/v) final DMSO concentration and 40-50% (v/v) final propylene glycol concentration to solubilise the payload. The solution was mixed for 5 hours at room temperature, after which the conjugation was quenched by addition of N-acetyl-L-cysteine (8 micromoles, 80 μL at 100 mM). The conjugation mixture was purified by spin filtration using a 15 mL Amicon Ultracell 50 kDa MWCO spin filter, then further purified by tangential flow filtration (TFF) to >50 diavolumes using a Spectrum Labs KrosFlo Research Ili system with a 30 kDa MWCO, 115 cm² surface area hollow fibre filter module at 50 mL/min in PBS with a transmembrane pressure (TMP) of 0.5-1.0 bar, and sterile-filtered and analysed.

UHPLC analysis on a Shimadzu Prominence system using a Phenomenex Aeris 3.6u XB-C18 150 mm x 2.1 mm column eluting with a gradient of water and acetonitrile on a reduced sample of ConjB (Tratuzumab-11) at 280 nm and 330 nm (Compound 11 specific) shows unconjugated light chains and a mixture of unconjugated heavy chains and heavy chains attached to a single molecule of Compound 11, consistent with a drug-per-antibody ratio (DAR) of 1.53 molecules of Compound 11 per antibody.

UHPLC analysis on a Shimadzu Prominence system using a Tosoh Bioscience TSKgel G3000SWXL 5 μm 7.8×300 mm column (with a 7 μm 6.0×40 mm guard column) eluting with sterile-filtered SEC buffer containing 200 mM potassium phosphate pH 6.95, 250 mM potassium chloride and 10% isopropanol (v/v) on a sample of ConjB (Tratuzumab-11) at 280 nm shows a monomer purity of over 95%. UHPLC SEC analysis gives a concentration of final ConjB (Tratuzumab-11) at 0.49 mg/mL in 19.0 mL, obtained mass of ConjB

(Tratuzumab-11) is 9.3 mg (62% yield).

ConjC, Tratuzumab-11

A 50 mM solution of tris(2-carboxyethyl)phosphine hydrochloride (TCEP) in phosphate-buffered saline pH 7.4 (PBS) was added (50 molar equivalent/antibody, 10.0 micromoles, 100 μL) to a 6.9 mL solution of antibody (30 mg, 200 nanomoles) in reduction buffer containing PBS and 1 mM ethylenediaminetetraacetic acid (EDTA) and a final antibody concentration of 4.35 mg/mL. The reduction mixture was heated at +37 ° C. for 3 hours (or until full reduction is observed by UHPLC) in an incubator with gentle (135 rpm) shaking. After cooling down to room temperature, the reduced antibody was buffer exchanged, via spin filter centrifugation, into a reoxidation buffer containing PBS pH 7.4 and 1 mM EDTA to remove all the excess reducing agent. A 50 mM solution of dehydroascorbic acid (DHAA, 13 molar equivalent/antibody, 2.6 micromoles, 52 μL) in DMSO was added and the reoxidation mixture was allowed to react for 16 hours at room temperature with gentle (<150 rpm) shaking at an antibody concentration of 2 mg/mL (or more DHAA added and reaction left for longer until full reoxidation of the cysteine thiols to reform the inter-chain cysteine disulfides is observed by UHPLC). The reoxidation mixture was then sterile-filtered and diluted in a conjugation buffer containing PBS pH 7.4, 1 mM EDTA for a final antibody concentration of 1.0-1.5 mg/mL. Compound 16 was added as a solution in DMSO (12.5 molar equivalent/antibody, 1.25 micromoles, in 1.0 mL DMSO) to 9 mL of this reoxidised antibody solution (15 mg, 100 nanomoles) for a 10% (v/v) final DMSO concentration. The solution was mixed for 1.25 hours at room temperature, after which the conjugation was quenched by addition of N-acetyl-L-cysteine (8 micromoles, 80 μL at 100 mM). The conjugation mixture was purified by spin filtration using a 15 mL Amicon Ultracell 50 kDa MWCO spin filter, then further purified by tangential flow filtration (TFF) to >50 diavolumes using a Spectrum Labs KrosFlo Research Ili system with a 30 kDa MWCO, 115 cm² surface area hollow fibre filter module at 50 mL/min in PBS with a transmembrane pressure (TMP) of 0.5-1.0 bar, and sterile-filtered and analysed. UHPLC analysis on a Shimadzu Prominence system using a Phenomenex Aeris 3.6u XB-C18 150 mm×2.1 mm column eluting with a gradient of water and acetonitrile on a reduced sample of ConjC (Tratuzumab-16) at 280 nm and 330 nm (Compound 16 specific) shows unconjugated light chains and a mixture of unconjugated heavy chains and heavy chains attached to a single molecule of Compound 16, consistent with a drug-per-antibody ratio (DAR) of 1.64 molecules of Compound 16 per antibody. UHPLC analysis on a Shimadzu Prominence system using a Tosoh Bioscience TSKgel G3000SWXL 5μm 7.8×300 mm column (with a 7 μm 6.0×40 mm guard column) eluting with sterile-filtered SEC buffer containing 200 mM potassium phosphate pH 6.95, 250 mM potassium chloride and 10% isopropanol (v/v) on a sample of ConjC (Tratuzumab-16) at 280 nm shows a monomer purity of 91%. UHPLC SEC analysis gives a concentration of final ConjC (Tratuzumab-16) at 0.57 mg/mL in 18.5 mL, obtained mass of ConjC (Tratuzumab-16) is 10.6 mg (71% yield).

Example 3-In Vitro Testing

Medium from sub-confluent (80-90% confluency) cell culture in a T75 flask was aspirated and the flask rinsed with PBS (about 20 ml) and emptied. Trypsin-EDTA (5 ml) was added, the flask returned to a 37° C., CO₂ (5%)-gassed incubator for up to about 5 minutes, then rapped sharply to dislodge and dissociate cells from the plastic. The cell suspension was transferred to a sterile 50 ml screw-top centrifuge tube, diluted with growth medium to a final volume of 15 ml, then centrifuged (400 g for 5 min). The supernatant was aspirated and the pellet re-suspended in 10 ml culture medium. Repeated pipetting may be necessary to produce monodisperse cell suspensions. The cell concentration and viability are measured by trypan blue staining, and counting using the LUNA-II™ Automated Cell Counter. Cells were diluted to 2×10⁵/ml, dispensed (50 μl /well) into 96-well flat-bottom plates and incubated overnight before use.

A stock solution (1 ml) of antibody drug conjugate (ADC) (20 μg/ml) was made by dilution of filter-sterilised ADC into cell culture medium. A set of 8×10-fold dilutions of stock ADC were made in a 24-well plate by serial transfer of 100 μl into 900 μl of cell culture medium. ADC dilution was dispensed (50 μl/well) into 4 replicate wells of the 96-well plate, containing 50 μl cell suspension, seeded the previous day. Control wells received 50 μl cell culture medium. The 96-well plate containing cells and ADCs was incubated at 37° C. in the CO2-gassed incubator for the exposure time.

At the end of the incubation period, cell viability was measured by MTS assay. MTS (Promega) was dispensed (20 μl per well) into each well and incubated for 4 hours at 37° C. in the CO₂-gassed incubator. The optical absorbance of each well was measured at 490 nm. Percentage cell survival was calculated from the mean absorbance in the 4 ADC-treated wells compared to the mean absorbance in the 4 control, untreated wells (100%).

The IC₅₀ was determined from the dose-response data using a non-linear curve fit algorithm: sigmoidal, 4PL X is log₁₀ (ADC concentration) in GraphPad Prism.

| | | ADC | Cell growth |
|---|---|---|---|
| Cell Line | Description | Exposure | medium |
| MDA-MB-468 | HER2 negative, | 4 days | RPMI 1640 with |
| ATCC HTB-132 | Breast carcinoma | | glutamax, 10% FBS |
| SK-BR-3 | Breast carcinoma | 4 days | McCoy's 5A with |
| ATCC HTB-30 | | | Glutamax, 10% FBS |
| NCI-N87 | Gastric carcinoma | 7 days | RPMI 1640 with |
| ATCC CRL-5822 | | | glutamax, 10% FBS |

| | Cytotoxicity IC₅₀ (nM) | | |
|---|---|---|---|
| Cell line | ConjA | ConjB | ConjC |
| NCI-N87 | 0.007548 | 0.1064 | 0.01479 |
| SK-BR-3 | 0.06795 | 0.1362 | 0.05614 |
| MDA MB 468 | >67 | >67 | >67 |

Example 4-Xenograft Testing

Mice

Female severe combined immune-deficient mice (Fox Chase SCID®, C.B-17/Icr-Prkdcscid, Charles River) were ten weeks old with a body weight (BW) range of 16.2 to 21.9 grams on Day 1 of the study. The animals were fed ad libitum water (reverse osmosis, 1 ppm Cl), and NIH 31 Modified and Irradiated Lab Diet® consisting of 18.0% crude protein, 5.0% crude fat, and 5.0% crude fibre. The mice were housed on irradiated Enricho'cobs™ Laboratory Animal Bedding in static micro-isolators on a 12-hour light cycle at 20-22° C. (68-72° F.) and 40-60% humidity. CR Discovery Services specifically complies with the recommendations of the Guide for Care and Use of Laboratory Animals with respect to restraint, husbandry, surgical procedures, feed and fluid regulation, and veterinary care. The animal care and use program at CR Discovery Services is accredited by the Association for Assessment and Accreditation of Laboratory Animal Care International (AAALAC), which assures compliance with accepted standards for the care and use of laboratory animals.

NCI-N87 Xenografts

Tumour Cell Culture

Human NCI-N87 gastric carcinoma lymphoma cells were cultured in RPMI-1640 medium supplemented with 10% fetal bovine serum, 2 mM glutamine, 100 units/mL penicillin, 100 pg/mL streptomycin sulfate and 25 pg/mL gentamicin. The cells were grown in tissue culture flasks in a humidified incubator at 37° C., in an atmosphere of 5% CO2 and 95% air.

In Vivo Implantation and Tumour Growth The NCI-N87 cells used for implantation were harvested during log phase growth and re-suspended in phosphate buffered saline (PBS) containing 50% Matrigel™ (BD Biosciences). On the day of tumour implant, each test mouse was injected subcutaneously in the right flank with 1×10⁷ cells (0.1 ml cell suspension), and tumour growth was monitored as the average size approached the target range of 100 to 150 mm³. Fourteen days later, designated as Day 1 of the study, mice were sorted according to calculated tumour size into groups each consisting of ten animals with individual tumour volumes ranging from 108 to 144 mm³ and group mean tumour volumes of 124-126 mm³. Tumours were measured in two dimensions using calipers, and volume was calculated using the formula:

$$Tumour Volume ( {mm}^{3} ) = \frac{{w}^{2} \times l}{2}$$

where w=width and l=length, in mm, of the tumour. Tumour weight may be estimated with the assumption that 1 mg is equivalent to 1 mm³ of tumour volume.

Treatment

Treatment began on Day 1 in groups of 10 mice (n=10) with established subcutaneous NCI-N87 tumours (range 108-144 mm³). Agents were administered intravenously once on Day 1 (qd×1). A vehicle-treated group served as the control group for efficacy analysis. Tumours were measured twice per week until the study was ended on Day 81. Each mouse was euthanized when its tumour reached the endpoint volume of 800 mm³ or on the final day, whichever came first. The time to endpoint (TTE) was calculated for each mouse.

Treatment outcome was determined from percent tumour growth delay (% TGD), defined as the percent increase in median TTE for treated versus control mice, with differences between groups deemed statistically significant at P≤0.05 using logrank survival analysis. Mice were monitored for complete regression (CR) and partial regression (PR) responses.

Treatment tolerability was assessed by body weight measurements and frequent observation for signs of treatment-related side effects. Treatment tolerability was assessed by body weight measurements and frequent observation for signs of treatment-related side effects. All regimens were well tolerated. The median TTE for vehicle-treated controls was 40.7 days, establishing a maximum possible TGD of 40.3 days (99%) for the 81-day study.

Both ConjA ADC treatment regimens tested produced significant survival benefit compared to vehicle-treated controls (Logrank (Mantel-Cox) Test P<0.001) and resulted in the maximum possible TGD. The 0.3 mg/kg group had a single PR regression response. The 1.0 mg/kg group had 40% regression responses consisting of three PRs and one CR which remained a TFS at study end. The 0.3 and 1.0 mg/kg groups had 5/9 and 10/10 81-day survivors, respectively with MTVs of 650 and 304 mm³.

ConjB at 0.3 mg/kg produced no survival benefit compared to vehicle-treated controls (P=0.2463) but at 1.0 mg/kg produced a significant survival benefit compared to vehicle-treated controls (P=0.0057). There were no tumour regression responses in either group. Only the 1.0 mg/kg group had 81-day survivors (2/10 with an MTV of 625 mm³).

ConjC at 0.3 mg/kg and 1.0 mg/kg produced significant survival benefit compared to vehicle-treated controls (P=0.0013 and P<0.001 respectively). The median TTEs for the 0.3 and 1.0 mg/kg groups were 65.6 and 81.0 days, corresponding to TGDs of 24.9 days (61%) and 40.3 days (99%). The 0.3 mg/kg group had one regression response (PR) whereas the 1.0 mg/kg group had 5/9 regressions consisting of five PRs. The 0.3 and 1.0 mg/kg groups had 3/10 and 9/9 81-day survivors, respectively, with MTVs of 486 and 75 mm³.

The minimum effective dose was defined as the minimum dosage required to produce tumour stasis for twenty-eight days after test article administration. Based on visual inspection of the mean and median tumour volume plots and percent change of tumour volume from Day 1, ConjA at 0.3 mg/kg and ConjC at 1.0 mg/kg, appeared to be the dosages that achieved a response consistent with the minimum effective dose. The minimum effective dose of ConjB was not determined in this regimen.

The results are illustrated in FIG. 1.

A further study was carried out with ConjB. Mice and xenografts were as described above for Study 1. Grafted mice were sorted into groups of ten animals with individual tumour volumes ranging from 108 to 172 mm³ and group mean tumour volumes of 133 mm³.

Both regimens were acceptably tolerated. The median TTE for the vehicle control group was 45.2 days, establishing a maximum possible TGD of 32.8 days (73%) for the 78-day study. The median TTE for both ConjB treated groups was 78.0 days, corresponding to the maximum possible TGD. The 3 mg/kg group had 80% regressions consisting of six PRs and two CRs; there were no TFS at study end. The 5 mg/kg group had 100% regression responses consisting of two PRs and eight CRs, six of which remained TFSs at study end. The 3.0 and 5.0 g/kg groups each had ten 78-day survivors with MTVs of 126 and 5 mm³ respectively.

The results are illustrated in FIG. 2.

The minimum effective dose was defined as the minimum dosage required to produce tumour stasis for twenty-eight days after test article administration. Based on visual inspection of the mean and median tumour volume plots and percent change of tumour volume from Day 1, the minimum effective dose of ConjB was not determined in this regimen. However, give the results of this and the previous study the minimum effective dose of ConjB is between 1 and 3 mg/kg.

JIMT-1 Xenografts

Tumour Cell Culture

JIMT-1 human breast carcinoma cells were cultured in Dulbecco's Modified Eagle's Medium (DMEM) containing 10% fetal bovine serum, 2 mM glutamine, 100 units/ml penicillin G sodium, 100 μg/mL streptomycin sulfate, and 25 μg/mL gentamicin. The cells were cultured in tissue culture flasks in a humidified incubator at 37° C., in an atmosphere of 5% CO₂ and 95% air.

In Vivo Implantation and Tumour Growth

On the day of implant, JIMT-1 cells were harvested during log phase growth and resuspended in phosphate buffered saline (PBS) at a concentration of 1×10⁸ cells/ml in 50% Matrigel™ (BD Biosciences). Xenografts were initiated by subcutaneously implanting 1×10⁷JIMT-1 cells (0.1 ml suspension) into the right flank of each test animal. Tumours were monitored as their volumes approached the target range of 100 to 150 mm³ and were measured in two dimensions using calipers. Tumour volume was calculated using the formula:

$$Tumour Volume ( {mm}^{3} ) = \frac{{w}^{2} \times l}{2}$$

where w=width and 1=length, in mm, of the tumour. Tumour weight may be estimated with the assumption that 1 mg is equivalent to 1 mm³ of tumour volume.

Treatment Fourteen days after tumour implantation, designated as Day 1 of the study, the animals were sorted into groups (n=10) with individual tumour volumes of 75 to 162 mm³ and group mean tumour volumes of 115 to 117 mm³. ConjC was administered intravenously in a single dose of 0.3 or 1 mg/kg on Day 1. A vehicle-treated group served as tumour growth controls. Tumours were measured twice per week until the study was ended on Day 64. Each mouse was euthanized when its tumour reached the endpoint volume of 1000 mm³ or on the final day, whichever came first. The time to endpoint (TTE) was calculated for each mouse.

Treatment outcome was determined from percent tumour growth delay (% TGD), defined as the percent increase in median TTE for treated versus control mice, with differences between groups deemed statistically significant at P≤0.05 using logrank survival analysis. Mice were monitored for complete regression (CR) and partial regression (PR) responses.

An animal with a CR at study end was additionally classified as a tumour-free survivor

(TFS). Treatment tolerability was assessed by body weight measurements and frequent observation for signs of treatment-related side effects.

Both regimens were acceptably tolerated. The median TTE for controls was 55.7 days, establishing a maximum possible TGD of 8.3 days (15%) for the 64-day study. Both ADC regimens evaluated produced significant survival benefit compared to control (P<0.001) and a maximal 15% TGD.

The 0.3 mg/kg group showed median tumour sizes that remained static throughout the study, had no objective regression responses but all animals remained end of study survivors with an MTV of 158 mm³.

The 1.0 mg/kg group showed a reduction in tumour size which remained throughout the study and produced 90% regressions consisting of three PRs and six CRs, five of which were classified as TFS at study end. All ten of the 1.0 mg/kg group animals remained end of study survivors with an MTV of 9 mm³.

The minimum effective dose was defined as the minimum dosage required to produce tumour stasis for twenty-eight days after test article administration. Based on visual inspection of the mean and median tumour volume plots and percent change of tumour volume from Day 1, the minimum effective dose of ConjC was 0.3 mg/kg.

The results are illustrated in FIG. 3.

Example 5-Toxicity studies/Therapeutic Index

A single dose toxicity study can be used to determine the maximum tolerated dose (MTD) and safety profile of the conjugtaes. Male Sprague Dawley rats (Harlan, Inc) may be dosed once by slow bolus intravenous injection via the tail vein with vehicle control (25 mM Histidine-HCl, 7% sucrose, 0.02% Polysorbate 80, pH 6.0) or test material. Parameters that may be evaluated during the study included mortality, physical examinations, cageside observations, body weights, body weight changes, clinical pathology (clinical chemistry, hematology, and coagulation), and gross pathology findings.

Tolerability can be determined based on toxicity end points, including body weight loss (>10%) and bone marrow suppression.

Therapeutic Index

The Therapeutic Index can be calculated by dividing the maximum tolerated single dose (MTD) of non-targeted ADC in rat, by the minimal effective single dose (MED) of the a targeted ADC. The MED is the single dose necessary to achieve tumour stasis in an in vivo model at 28 days (for NCI-N87 xenograft).

All documents and other references mentioned above are herein incorporated by reference.

1. A conjugate of formula I:
L-(D^{L})ₚ (I)
wherein Lisa Ligand unit, D is a Drug Linker unit of formula IIa, IIb or IIc: 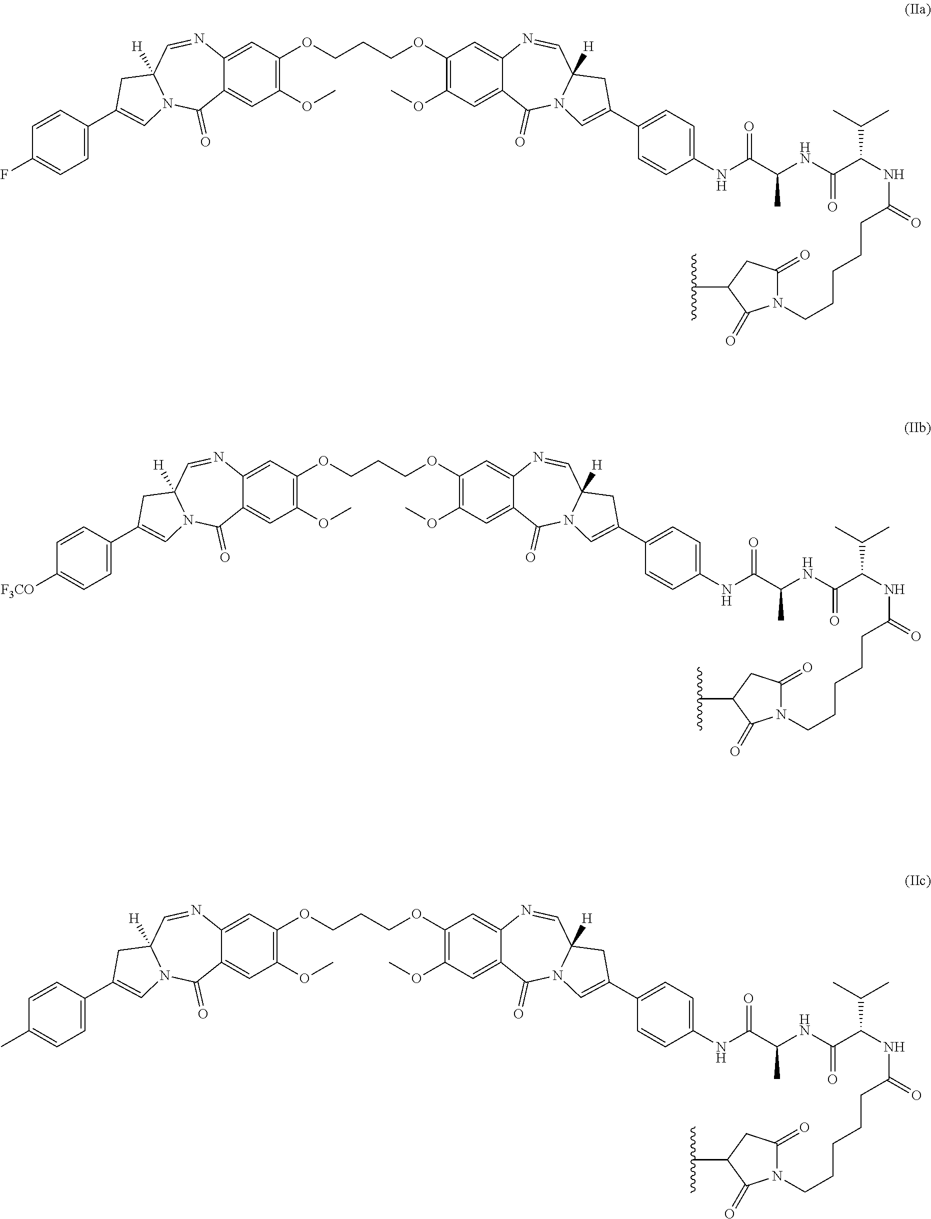
wherein p is an integer of from 1 to 20.

2. A conjugate according to claim 1, wherein the Ligand Unit is an antibody or an active fragment thereof.

3. The conjugate according to claim 2, wherein the antibody or antibody fragment is an antibody or antibody fragment for a tumour-associated antigen.

4. The conjugate according to claim 3 wherein the antibody or antibody fragment is an antibody which binds to one or more tumor-associated antigens or cell-surface receptors selected from (1)-(88):
(1) BMPR1B;
(2) E16;
(3) STEAP1;
(4) 0772P;
(5) MPF;
(6) Napi3b;
(7) Sema 5b;
(8) PSCA hlg;
(9) ETBR;
(10) MSG783;
(11) STEAP2;
(12) TrpM4;
(13) CRIPTO;
(14) CD21;
(15) CD79b;
(16) FcRH2;
(17) HER2;
(18) NCA;
(19) MDP;
(20) IL20R-alpha;
(21) Brevican;
(22) EphB2R;
(23) ASLG659;
(24) PSCA;
(25) GEDA;
(26) BAFF-R;
(27) CD22;
(28) CD79a;
(29) CXCR5;
(30) HLA-DOB;
(31) P2X5;
(32) CD72;
(33) LY64;
(34) FcRH1;
(35) IRTA2;
(36) TENB2;
(37) PSMA-FOLH1;
(38) SST;
(38.1) SSTR2;
(38.2) SSTR5;
(38.3) SSTR1;
(38.4)SSTR3;
(38.5) SSTR4;
(39) ITGAV;
(40) ITGB6;
(41) CEACAM5;
(42) MET;
(43) MUC1;
(44) CA9;
(45) EGFRvIII;
(46) CD33;
(47) CD19;
(48) IL2RA;
(49) AXL;
(50) CD30-TNFRSF8;
(51) BCMA-TNFRSF17;
(52) CT Ags-CTA;
(53) CD174 (Lewis Y)-FUT3;
(54) CLEC14A;
(55) GRP78-HSPAS;
(56) CD70;
(57) Stem Cell specific antigens;
(58) ASG-5;
(59) ENPP3;
(60) PRR4;
(61) GCC-GUCY2C;
(62) Liv-1-SLC39A6;
(63) 5T4;
(64) CD56 -NCMA1;
(65) CanAg;
(66) FOLR1;
(67) GPNMB;
(68) TIM-1-HAVCR1;
(69) RG-1/Prostate tumor target Mindin-Mindin/RG-1;
(70) B7-H4-VTCN1;
(71) PTK7;
(72) CD37;
(73) CD138-SDC1;
(74) CD74;
(75) Claudins-CLs;
(76) EGFR;
(77) Her3;
(78) RON-MST1R;
(79) EPHA2;
(80) CD20-MS4A1;
(81) Tenascin C-TNC;
(82) FAP;
(83) DKK-1;
(84) CD52;
(85) CS1- SLAMF7;
(86) Endoglin-ENG;
(87) Annexin A1-ANXA1;
(88) V-CAM (CD106)-VCAM1.

5. The conjugate of claim 2, wherein the antibody or antibody fragment is a cysteine-engineered antibody.

6. The conjugate of claim 1, wherein p is an integer from 1 to 8.

7. The conjugate according to claim 6, wherein p is 1, 2, 3, or 4.

8. A composition comprising a mixture of conjugates according to claim 1, wherein the average p in the mixture of conjugate compounds is about 1 to about 8.

9. The conjugate of claim 1, for use in therapy.

10. A pharmaceutical composition comprising the conjugate of of claim 1 and a pharmaceutically acceptable diluent, carrier, or excipient.

11.-13. (canceled)

14. A method of medical treatment comprising administering to a patient the pharmaceutical composition of claim 10.

15. The method of claim 14 wherein the method of medical treatment is for treating cancer.

16. The method of claim 15, wherein the patient is administered a chemotherapeutic agent, in combination with the conjugate.

17. (canceled)

18. A method of treating a mammal having a proliferative disease, comprising administering to the mammal an effective amount of a conjugate of claim 1.

19. A compound of formula IIIa, IIIb or IIIc:

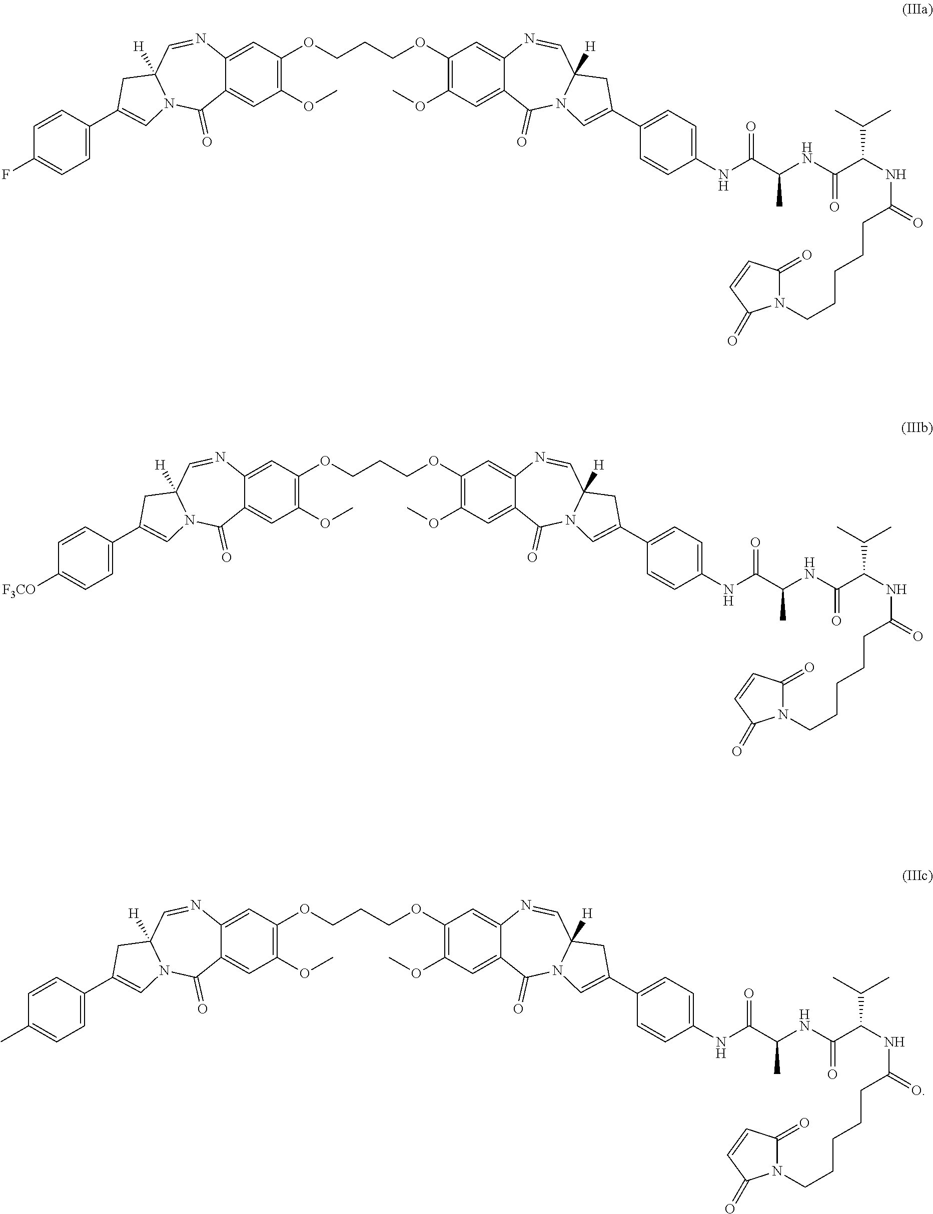 [SMILES: COc1cc2c(cc1OCCCOc1cc3c(cc1OC)C(=O)N1C=C(c4ccc(NC(=O)[C@H](C)NC(=O)[C@@H](NC(=O)CCCCCN5C(=O)C=CC5=O)C(C)C)cc4)C[C@H]1C=N3)N=C[C@@H]1CC(c3ccc(F)cc3)=CN1C2=O ; COc1cc2c(cc1OCCCOc1cc3c(cc1OC)C(=O)N1C=C(c4ccc(OC(F)(F)F)cc4)C[C@H]1C=N3)N=C[C@@H]1CC(c3ccc(NC(=O)[C@H](C)NC(=O)[C@@H](NC(=O)CCCCCN4C(=O)C=CC4=O)C(C)C)cc3)=CN1C2=O ; COc1cc2c(cc1OCCCOc1cc3c(cc1OC)C(=O)N1C=C(c4ccc(NC(=O)[C@H](C)NC(=O)[C@@H](NC(=O)CCCCCN5C(=O)C=CC5=O)C(C)C)cc4)C[C@H]1C=N3)N=C[C@@H]1CC(c3ccc(C)cc3)=CN1C2=O]

20. A method of synthesizing a conjugate of formula I:
L-(D^{L})ₚ (I)
wherein Lisa Ligand unit, D is a Drug Linker unit of formula IIa, IIb or IIIc: 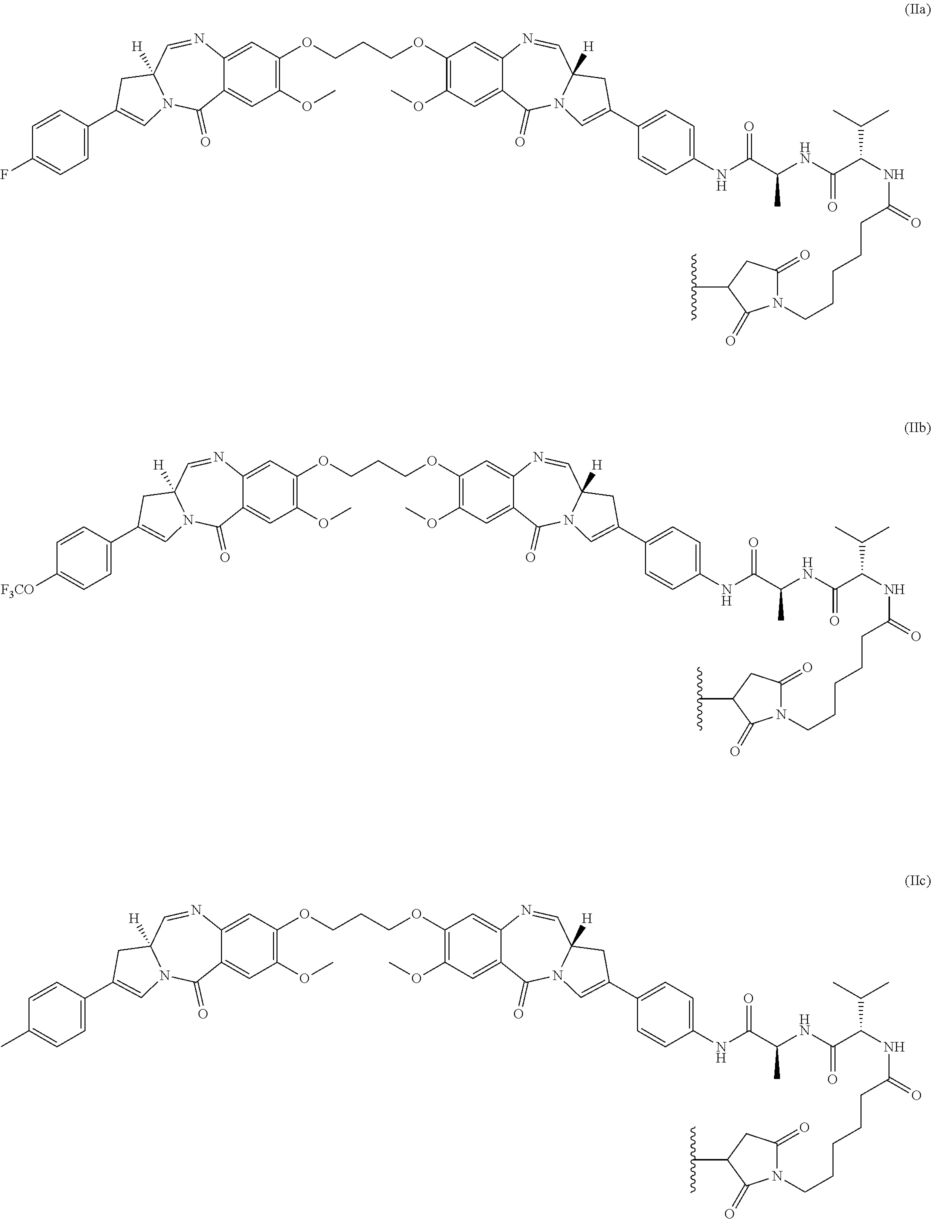
wherein p is an integer of from 1 to 20, which comprises conjugating a compound according to claim 19 with a cell-binding agent.

21. A method of treating a mammal having a proliferative disease, comprising administering to the mammal an effective amount of a pharmaceutical composition according to claim 10.
